(19) United States
(12) Patent Application Publication
(10) Pub. No.: US 2012/0095268 A1
(43) Pub. Date: Apr. 19, 2012

(54) MICROCHANNEL PROCESSOR

(76) Inventors: Anna Lee Tonkovich, Dublin, OH (US); Thomas Yuschak, Lewis Center, OH (US); Kai Tod Paul Jarosch, Bexley, OH (US); Paul Neagle, Westerville, OH (US); Bin Yang, Columbus, OH (US); Ravi Arora, New Albany, OH (US); Jeffrey Marco, London, OH (US); Jennifer Marco, London, OH (US); Barry L. Yang, Dublin, OH (US); Andreas Munding, Madison, AL (US); Sara Kampfe, Dublin, OH (US)

(21) Appl. No.: 13/275,727

(22) Filed: Oct. 18, 2011

Related U.S. Application Data
(60) Provisional application No. 61/510,191, filed on Jul. 21, 2011.
(60) Provisional application No. 61/441,276, filed on Feb. 9, 2011.
(60) Provisional application No. 61/394,328, filed on Oct. 18, 2010.

(51) Int. Cl. B01J 19/00; B23P 6/00; C07C 11/167; C07C 11/04; C07C 15/02; B21D 53/02; C07C 45/00

(52) U.S. Cl. 568/449; 29/890.04; 29/890.031; 422/198; 422/601; 585/500; 585/400

(57) ABSTRACT

This invention relates to an apparatus, comprising: a plurality of plates in a stack defining at least one process layer and at least one heat exchange layer, each plate having a peripheral edge, the peripheral edge of each plate being welded to the peripheral edge of the next adjacent plate to provide a perimeter seal for the stack, the ratio of the average surface area of each of the adjacent plates to the average penetration of the weld between the adjacent plates being at least about 100 cm²/mm. The stack may be used as the core assembly for a microchannel processor. The microchannel processor may be used for conducting one or more unit operations, including chemical reactions such as SMR reactions.

This application claims priority under 35 U.S.C. §119(e) to U.S. Provisional Application Ser. No. 61/510,191, filed Jul. 21, 2011; U.S. Provisional Application Ser. No. 61/441,276, filed Feb. 9, 2011; and U.S. Provisional Application Ser. No. 61/394,328, filed Oct. 18, 2010. The disclosures in these provisional applications are incorporated herein by reference.

### TECHNICAL FIELD

This invention relates to microchannel processors and, more particularly, to microchannel processors that can be refurbished.

### BACKGROUND

The conventional thinking in microchannel technology has been that optimal heat transfer in a microchannel processor could only be obtained by brazing or diffusion bonding. These methods rely on the formation of a contiguous metallic interface between the layers. The contiguous interface may be advantageous for the purposes of heat transfer to move heat from an exothermic reaction to heat removal layers or to add heat to an endothermic reaction.

### SUMMARY

A problem with microchannel processors made using brazing or diffusion bonding to provide a contiguous metallic interface between the layers is that they are not readily adaptable to disassembly and refurbishment, which typically includes replacement of catalyst coatings as well as other coatings, such as protective barrier coatings, non-stick coatings, coatings that are resistant metal dusting, corrosion inhibiting coatings, and the like. Thus, these processors typically require replacement when used over extended periods of use. Microchannel processors can be costly and the requirement for replacement over extended periods of use is commercially unacceptable for many applications. The present invention provides a solution to this problem.

This invention relates to an apparatus which may be used as the core assembly for a microchannel processor. The apparatus may comprise: a plurality of plates in a stack defining at least one process layer and at least one heat exchange layer, each plate having a peripheral edge, the peripheral edge of each plate being welded to the peripheral edge of the next adjacent plate to provide a perimeter seal for the stack, the ratio of the average surface area of each of the adjacent plates in square centimeters (cm²) to the average penetration of the weld between the adjacent plates in millimeters (mm) being at least about 100 cm²/mm, or in the range from about 100 to about 100,000, or from about 100 to about 50,000, or from about 100 to about 30,000, or from about 100 to about 20,000, or from about 100 to about 10,000, or from about 100 to about 5000, or from about 100 to about 2000, or from about 100 to about 1800, or from about 100 to about 1600 cm²/mm. These ratios may be significant since it was unexpected that relatively large microchannel processors using peripheral welding with plate surface area to weld penetration ratios in these ranges could be successfully used.

This invention relates to an apparatus, comprising: a plurality of plates in a stack defining at least one process layer and at least one heat exchange layer, each plate having a peripheral edge, the peripheral edge of each plate being welded to the peripheral edge of the next adjacent plate to provide a perimeter seal for the stack, the process layer containing a steam methane reforming catalyst, the heat exchange layer containing a combustion catalyst.

In an embodiment, the stack may be positioned in a containment vessel, the stack being adapted to operate at an internal pressure above atmospheric pressure, the containment vessel being adapted to operate at an internal pressure above atmospheric pressure and provide for the application of pressure to the exterior surface of the stack, the containment vessel including a control mechanism to maintain a pressure within the containment vessel at least as high as the internal pressure within the stack. The control mechanism may comprise a check valve and/or a pressure regulator. In an embodiment, a reactant gas may be used in the process layer and a contaminant gas may be used in the containment vessel, the control mechanism including a piping system to divert process gas to the interior of the containment vessel in the event the pressure provided by the containment gas decreases.

In an embodiment, an exoskeleton may be mounted on the exterior of the stack to provide structural support for the stack.

In an embodiment, end plates may be attached to each side of the stack to provide structural support for the stack.

In an embodiment, the process layer may comprise at least one process microchannel for conducting a unit operation, and the heat exchange layer may comprise at least one channel containing a heat exchange fluid, wherein the heat exchange fluid provides heating or cooling for the process layer.

In an embodiment, the process layer may comprise a plurality of process microchannels formed in a plate, the apparatus including internal welding to prevent the flow of fluid from one process microchannel to another process microchannel in the same plate.

In an embodiment, the heat exchange layer may comprise a plurality of heat exchange channels formed in a plate, the apparatus including internal welding to prevent the flow of fluid from one heat exchange channel to another heat exchange channel in the same plate.

In an embodiment, a welding material may be used to weld the peripheral edge of each plate, the plates being made of a metal or metal alloy, and the welding is material being made of a metal or metal alloy. In an embodiment, the plates and the welding material may be made of the same metal or metal alloy. In an embodiment, the metal alloy may comprise nickel, chromium, cobalt, molybdenum and aluminum.

In an embodiment, the peripheral edge of each plate may be welded to the peripheral edge of the next adjacent plate using a laser.

The plates may have surface areas of at least about 200 square centimeters (cm²), or from about 200 to about 48000 cm², or from about 200 to about 30,000, or from about 200 to about 15000, or from about 1000 to about 5000, or from about 1500 to about 2500, or about 2000 cm². The term “surface area” of a plate refers to the product of the overall length of the plate multiplied by the overall width of the plate. Thus, for example, a plate having an overall length of 75 cm and an overall width of 30 cm will have a surface area of 2250 cm².

The average penetration of the weld between the adjacent plates may up to about 10 millimeters (mm), or from about 0.25 to about 10 mm, or from about 0.25 to about 8 mm, or from about 0.25 to about 6.5 mm, or from about 0.25 to about 5 mm, or from about 0.5 to about 3 mm, or from about 0.75 to about 3 mm, or from about 1 to about 2 mm, or from about 1 to about 1.5 mm, or about 1.27 mm. The term “average penetration of a weld” refers to the average depth a welding material penetrates the gap between two adjacent plates when the welding material is applied to the peripheral edges of two adjacent plates. This is illustrated in FIG. 22 wherein a weld is applied to the peripheral edge of two adjacent plates, and the weld penetrates (“Weld Penetration”) the gap between the plates.

The apparatus may comprise a sufficient number of plates to provide for one or a plurality of process layers, for example, from 1 to about 1000, or from 1 to about 100, or from 1 to about 50, or from 1 to about 30, or from about 2 to about 30, or from about 4 to about 30, or from about 8 to about 24, or about 16 process layers; and one or a plurality of heat exchange layers, for example, from 1 to about 1000, or from 1 to about 100, or from 1 to about 50, or from 1 to about 30, or from about 2 to about 30, or from about 4 to about 36, or from about 8 to about 24, or about 16 heat exchange layers. The plates may be aligned horizontally and stacked one above another, aligned vertically and positioned side-by-side, or they may be aligned at an angle to the horizontal. The process layers and heat exchange layers may be aligned in alternating sequence with a process layer adjacent to a heat exchange layer, which in turn is adjacent to another process layer, which in turn is adjacent to another heat exchange layer, etc. Alternatively, two or more process layers and/or two or more heat exchange layers may be positioned adjacent to one another.

The apparatus may comprise one or plurality of repeat units, wherein each repeat unit is the same and each comprises one or more process layers and one or more heat exchange layers. For example, a repeat unit may comprise from 1 to about 10, or from 1 to about 5, or from 1 to about 3, or about 2 process layers; and from 1 to about 10, or from 1 to about 5, or from 1 to about 3, or about 2 heat exchange layers. The repeat units may be aligned horizontally and stacked one above another, aligned vertically and positioned side-by-side, or they may be aligned at an angle to the horizontal. Within each repeat unit the process layers and heat exchange layers may be aligned in alternating sequence with a process layer adjacent to a heat exchange layer, which in turn is adjacent to another process layer, which in turn is adjacent to another heat exchange layer, etc. Alternatively, two or more process layers and/or two or more heat exchange layers may be positioned adjacent to one another. The stack of plates may comprise any number of repeat units, for example, from 1 to about 1000, or from 1 to about 500, or from 1 to about 100, or from 1 to about 50, or from 1 to about 20, or from 1 to about 10 repeat units.

The apparatus may further comprise: an inlet process manifold welded to the stack to provide for the flow of fluid into the process layer; an outlet process manifold welded to the stack to provide for the flow of fluid out of the process layer; at least one inlet heat exchange manifold welded to the stack to provide for the flow of fluid into the heat exchange layer; and a heat exchange outlet to provide for the flow of fluid out of the heat exchange layer. The heat exchange outlet may comprise an exhaust outlet welded to an end of the stack and adapted to provide for the flow of exhaust gas from the heat exchange layer.

As indicated above, the stack, which may be referred to as a core assembly, may be placed in a containment vessel or have mechanical braces placed around the core assembly to withstand pressure during operation. The stack may be adapted to operate at an internal pressure above atmospheric pressure, for example, a gauge pressure up to about 15 MPa, or up to about 12 MPa, or up to about 10 MPa, or up to about 7 MPa, or up to about 5 MPa, or up to about 3 MPa, or is in the range from about 0.1 to about 15 MPa, or in the range from about 0.1 to about 12 MPa, or in the range from about 0.1 to about 10 MPa, or in the range from about 0.1 to about 7 MPa, or in the range from about 0.1 to about 5 MPa, or in the range from about 0.1 to about 3 MPa, or in the range from about 0.2 to about 10 MPa, or in the range from about 0.2 to about 5 MPa. The internal pressure within the stack may be generated by process activity in the process layer and/or heat exchange activity in the heat exchange layer. There may be two or more internal pressures within the stack as a result of operating a first unit operation at a first pressure in the process layer and a heat exchange process at a second pressure in the heat exchange layer. For example, a relatively high pressure may result from a high pressure reaction, such as an SMR reaction, in the process layer and a relatively low pressure reaction, such as a combustion reaction in the heat exchange layer. The difference in pressure between the internal pressure in the process layer and the internal pressure in the heat exchange layer may be up to about 10 MPa, or in the range from about 0.1 to about 10 MPa, or from about 0.2 to about 5 MPa. The containment vessel may also be adapted to operate at an internal pressure above atmospheric pressure, for example, a gauge pressure up to about 10 MPa, or up to about 7 MPa, or up to about 5 MPa, or up to about 4 MPa, or up to about 3.5 MPa, or up to about 3 MPa, or in the range from about 0.1 to about 10 MPa, or in the range from about 0.1 to about 7 MPa, or in the range from about 0.1 to about 5 MPa, or in the range from about 0.1 to about 3 MPa. The internal pressure within the containment vessel may be maintained using a containment gas. The containment gas may be an inert gas such as nitrogen. The internal pressure within the containment vessel may be used to provide pressure against the exterior surface of the stack, and thereby provide structural support for the stack. As indicated above, the containment vessel may include a control mechanism to maintain the pressure within the containment vessel at a level at least as high as the internal pressure within the stack. In this way, the pressure exerted on the exterior of the stack may at least equalize, or may exceed, the internal pressure within the stack. Because of the structural support provided by the containment gas, the use of clamps, external braces, external supports, and the like, for providing structural support for the stack may be avoided. The clamps, external braces, external supports, and the like, may be costly and problematic when refurbishment is desired.

As indicated above, the control mechanism for maintaining pressure within the containment vessel may comprise a check valve and/or a pressure regulator. Either or both of these may be used in combination with a system of pipes, valves, controllers, and the like, to ensure that the pressure in the containment vessel is maintained at a level that is at least as high as the internal pressure within the stack. This is done in part to protect the peripheral welds used to seal the stack. A significant decrease in the pressure within the containment vessel without a corresponding decrease of the internal pressure within the stack could result in a costly rupture of the peripheral welds. The control mechanism may include a piping system to allow for diversion of one or more process gases into the containment vessel in the event the pressure exerted by the containment gas decreases.

As indicated above, a structural support, which may comprise an exoskeleton, may be mounted on the exterior of the stack to provide structural support for the stack. The exoskeleton may comprise an array of stiffening members which are held (for example, via welding) in intimate contact with major exterior faces of the endplates of the stack. The stiffness of the members of the array may be such that they resist bending in the stacking direction (i.e., the direction orthogonal to the plane of the plates). Alternatively, there may also be stiffness members added in the plane of the plates to minimize a side or end rapture. The use of an exoskeleton for providing structural support for the stack is illustrated in FIG. 32.

As indicated above, a structural support may be provided by the use of relatively thick endplates attached or welded to each side of the stack. The relatively thick endplates may have a thickness of about one or more centimeters and may be sized based on the cross section of the stack along with the intended design temperature and pressure for the reactor. In the embodiment with relatively thick endplates to maintain the internal pressure during operation, the weld penetration for the endplates may be greater than the weld penetration used with the interior plates in the stack. As such, the weld penetration for the end plates may be greater than about 0.75 mm, or greater than about 1.5 mm, or greater than about 2 mm, or greater than about 3 mm, or greater than about 5 mm, or greater than about 7 mm, or greater than about 10 mm.

The apparatus may be suitable for conducing at least one unit operation in is the process layer. The unit operation may comprise a chemical reaction, vaporization, compression, chemical separation, distillation, condensation, mixing, heating, cooling, or a combination of two or more thereof.

The chemical reaction may comprise a methanol synthesis reaction, dimethyl ether synthesis reaction, ammonia synthesis reaction, water gas shift reaction, acetylation addition reaction, alkylation, dealkylation, hydrodealkylation, reductive alkylation, amination, aromatization, arylation, autothermal reforming, carbonylation, decarbonylation, reductive carbonylation, carboxylation, reductive carboxylation, reductive coupling, condensation, cracking, hydrocracking, cyclization, cyclooligomerization, dehalogenation, dimerization, epoxidation, esterification, Fischer-Tropsch reaction, halogenation, hydrohalogenation, homologation, hydration, dehydration, hydrogenation, dehydrogenation, hydrocarboxylation, hydroformylation, hydrogenolysis, hydrometallation, hydrosilation, hydrolysis, hydrotreating, isomerization, methylation, demethylation, metathesis, nitration, oxidation, partial oxidation, polymerization, reduction, reformation, reverse water gas shift, sulfonation, telomerization, transesterification, trimerization, Sabatier reaction, carbon dioxide reforming, preferential oxidation, partial oxidation, or preferential methanation reaction. The chemical reaction may comprise a steam methane reforming (SMR) reaction. The chemical reaction may comprise a process for making ethylene, styrene, formaldehyde and/or butadiene.

The process layer may comprise a plurality of process microchannels aligned in parallel. Each process microchannel may comprise a reaction zone containing a catalyst. The process layer may comprise a plurality of internal manifolds adapted to provide for a substantially uniform distribution of reactants flowing into the process microchannels. The process layer may also comprise a plurality of internal manifolds adapted to provide for a substantially uniform distribution of product flowing out of the process microchannels. The process microchannels may contain surface features and/or capillary features.

The process layer may comprise a reactant layer, a product layer, and a process u-turn positioned at an end of the reactant layer and product layer to allow for the flow of fluid from the reactant layer to the product layer. The reactant layer may be positioned adjacent to the product layer. The process layer may be adapted is for use in a reaction wherein one or more reactants react to form a product, the one or more reactants flowing into the reactant layer, contacting a catalyst and reacting to form a product, the product flowing out of the product layer.

The heat exchange layer may comprise a plurality of heat exchange channels aligned in parallel. The heat exchange channels may be used to provide heating or cooling for the process layer. The heat exchange channels may comprise microchannels. The heat exchange channels may contain surface features and/or capillary features. The heat exchange layer may be adapted to provide for the flow of a heat exchange fluid into, through and out of the heat exchange channels. The heat exchange fluid may comprise a liquid, a gas, or a mixture thereof. The heat exchange layer may be adapted for conducting in the heat exchange layer a combustion reaction or, alternatively, other oxidation or exothermic reactions, for example, partial oxidation reactions, and the like.

The heat exchange layer may comprise a fuel layer, an air layer positioned adjacent to the fuel layer, a heat exchange wall positioned between the fuel layer and the air layer, a plurality of openings or jets in the heat exchange wall to allow for the flow of air from the air layer into the fuel layer, a combustion catalyst positioned in the fuel layer, an exhaust layer, and a heat exchange u-turn positioned at an end of the fuel layer and an end of the exhaust layer to allow for the flow of exhaust from the fuel layer to the exhaust layer. The heat exchange layer may be adapted to allow for a fuel to flow in the fuel layer, air to flow from the air layer through the openings in the heat exchange wall into the fuel layer to combine with the fuel to form a fuel-air mixture, flowing the fuel-air mixture in contact with the combustion catalyst to provide for a combustion reaction to yield heat and an exhaust gas, the heat providing heat for the process layer, the exhaust gas flowing through the exhaust layer out of the heat exchange layer. The fuel layer may comprise a plurality of fuel microchannels and a plurality of internal manifolds adapted to provide for a substantially uniform distribution of fuel flowing into the fuel microchannels. The air layer may comprise a plurality of air microchannels and a plurality of internal manifolds adapted to provide for a substantially uniform distribution of air flowing into the air microchannels. The fuel layer and/or the air layer may contain surface features and/or capillary features.

The apparatus may comprise a steam methane reforming reactor, the is process layer containing a steam methane reforming catalyst, the heat exchange layer containing a combustion catalyst. The steam methane reforming catalyst may comprise rhodium and an alumina support. The combustion catalyst may comprise platinum, palladium and an alumina support, the alumina support being impregnated with lanthanum.

The apparatus may comprise a catalyst in the process layer and/or the heat exchange layer, the catalyst being applied to one or more plates ex-situ prior to welding the plates to form the stack.

The apparatus may comprise one or more plates that have an anti-corrosion and/or anti-sticking layer on one or more surfaces of such plates.

The apparatus may comprise one or more plates that have a metal dust resistant layer on one or more surfaces of such plates.

In an embodiment, one or more of the plates has one or more surface protection layers on it. In an embodiment, the surface protection layer comprises two or three layers, each layer comprising a different composition of materials. In an embodiment, the surface protection layer comprises three layers, the first layer comprising copper, the second layer comprising an aluminum-containing metal alloy, and the third layer comprising a metal alloy. In an embodiment, a catalyst is adhered to the surface protection layer.

The invention relates to a process for forming the foregoing apparatus, the process comprising: forming the stack of plates; and welding the peripheral edge of each plate to the peripheral edge of the next adjacent plate to provide the perimeter seal.

The invention relates to a process for refurbishing the foregoing apparatus, the process comprising: removing the welding from the peripheral edges of the plates; separating the plates; correcting defects in the plates; reforming the stack of plates; and welding the peripheral edge of each plate to the peripheral edge of the next adjacent plate to provide a new perimeter seal for the stack. The invention relates to a refurbished apparatus formed by the foregoing refurbishing process. This refurbishing process may be repeated any desired number of times, for example, from 1 to about 20 times, or from 1 to about 15 times, or from 1 to about 10 times, or from 1 to about 5 times, or from 1 to about 2 or 3 or 4 times, during the useful life of the apparatus. When the apparatus contains one or more catalysts, the is catalysts may be replaced and/or regenerated prior to reforming the stack of plates. When one or more catalysts are adhered to one or more surfaces of the plates, the catalyst may be removed by grit blasting. When one or more of the plates comprises an alumina scale that is damaged, the alumina scale may be replenished by heat treating. During refurbishment, one or more of the plates may be replaced and, as such, the apparatus after refurbishment may comprise one or more plates with different manufacturing dates. The replacement of one or more plates during refurbishment may result in a refurbished apparatus in which one or more of the plates are different than the original set of plates used previously. The replacement plate would require a slightly smaller cross section than the original plates to accommodate the metal loss from the original stack when the first weld sets are removed for refurbishment. The resulting new stack after refurbishment may have a slightly smaller cross section at each refurbishment cycle. It is expected that the amount of perimeter metal removed during each refurbishment cycle may range from about 0.1 mm to about 10 mm, or from about 0.5 to about 2 mm. Minimizing the amount of perimeter metal lost during each refurbishment cycle is preferred.

The peripheral welds may be relatively thin in order to facilitate refurbishment of the apparatus. For example, the average weld penetration may be up to about 10 mm, or from about 0.25 to about 10 mm, or from about 0.25 to about 8 mm, or from about 0.5 to about 6.5 mm, or from about 0.5 to about 5 mm, or from about 0.5 to about 3 mm, or from about 0.75 to about 2 mm, or from about 0.75 to about 1.5 mm, or about 0.05 inch (1.27 mm). Each of the plates may have a border surrounding the active area (e.g., process microchannels, heat exchange channels, etc.) of each plate. This is illustrated in FIG. 21. During refurbishment, the peripheral welding and part of the border may be removed, for example, by machining the weld and border. Thus, with thinner welds, less border material may be lost during each refurbishment. For example, if the average penetration of each weld is 0.05 inch (1.27 mm), and each border of each plate has a width of 0.5 inch (12.7 mm), each plate could be refurbished ten times before being discarded. This is significant since allowing for numerous refurbishments may significantly extend the useful life of a microchannel processor and thereby reduce its overall cost.

This invention relates to a process for conducting a unit operation using the above-indicated apparatus, comprising: conducting the unit operation in the process is layer; and exchanging heat between the process layer and the heat exchange layer.

This invention relates to a process for conducting a chemical reaction using the above-indicated apparatus, comprising: conducting the chemical reaction in the process layer; and exchanging heat between the process layer and the heat exchange layer.

This invention relates to a process for conducting a steam methane reforming reaction using the above-described apparatus, the process comprising: reacting steam with methane or natural gas in the presence of a catalyst in the process layer to form synthesis gas; and conducting a combustion reaction in the heat exchange layer to provide heat for the process layer.

In an embodiment for conducting the steam methane reforming reaction, the flow of methane or natural gas in the process layer is at a superficial velocity in the range from about 10 to about 200 meters per second, the approach to equilibrium for the steam methane reforming reaction being at least about 80%, and the reaction heat per pressure drop in the apparatus being in the range from about 2 to about 20 W/Pa.

In an embodiment for conducting the steam methane reforming reaction, the contact time for the steam methane reforming reaction is up to about 25 ms, the approach to equilibrium for the steam methane reforming reaction being at least about 80%, and the reaction heat per pressure drop in the apparatus being in the range from about 2 to about 20 W/Pa. In an embodiment, the reaction heat per unit contact time is at least about 20 W/ms. In an embodiment, the reaction heat per pressure drop in the apparatus is in the range from about 2 to about 20 W/Pa.

In an embodiment for conducting the steam methane reforming reaction in the inventive apparatus, the steam methane reforming reaction may be conducted for at least about 2000 hours without metal dusting pits forming on surfaces of the plates. In an embodiment, the steam methane reforming reaction is conducted for at least about 2000 hours and the pressure drop for the process layer after conducting the reaction for at least about 2000 hours increases by less than about 20% of the pressure drop at the start of the process.

In an embodiment, a plate in the process layer and/or heat exchange layer may comprise a surface wherein part, but not all, of the surface has a catalyst, anti-corrosion and/or anti-sticking layer, and/or metal dust resistant layer adhered to its surface. The apparatus may be a newly constructed apparatus or a refurbished apparatus. The foregoing catalyst, anti-corrosion and/or anti-sticking layer, and/or metal dust resistant layer may be referred to as being in the form of a discontinuous layer as compared to continuous layer wherein the entire plate would be covered. The application of such a discontinuous layer is feasible using the ex-situ coating method and the masked application techniques discussed below.

### BRIEF DESCRIPTION OF THE DRAWINGS

In the annexed drawings, like parts and features are accorded like designations.

FIG. 1 is a schematic illustration showing a stack of plates used to form the inventive apparatus. For purposes of illustration, some of the plates are stacked together, and others are shown as separated from the stack.

FIG. 2 is a schematic illustration showing the stack of plates from FIG. 1, in assembled form, and separate fluid manifolds to provide for the flow of process and heat exchange fluids into and out of the stack.

FIG. 3 is a schematic illustration of the stack of plates and fluid manifolds shown in FIG. 2, with the fluid manifolds welded to the stack to provide an assembled microchannel processor.

FIG. 4 is a schematic illustration of the assembled microchannel processor from FIG. 3 mounted in the header of a containment vessel.

FIG. 5 is a schematic illustration of a containment vessel used for housing the microchannel processor shown in FIGS. 3 and 4.

FIG. 6 is a schematic illustration showing the flow of reactants and product in the process layer of the inventive microchannel processor, and the flow of fuel, air and exhaust in the heat exchange layer of the inventive microchannel processor.

FIGS. 7 and 8 are schematic illustrations of a repeat unit comprising a stack of plates used in the inventive microchannel processor.

FIGS. 9-18 are schematic illustrations showing the top and bottom surfaces of each of the plates illustrated in FIGS. 7 and 8.

FIGS. 19 and 20 are photographs of a stack of plates of the type illustrated in FIGS. 1 to 4 with the peripheral edge of each plate welded to the peripheral edge of the next adjacent plate to provide a perimeter seal for the stack.

FIG. 21 is a schematic illustration of a portion of one of the plates illustrated in FIGS. 1 to 4 with an active area comprising a plurality of microchannels surrounded by a border, the border forming part of the peripheral edge of the late, and a weld applied to the peripheral edge of the plate and penetrating beyond the peripheral edge.

FIG. 22 is a schematic illustration of a portion of two plates of the type illustrated in FIGS. 1 to 4 with a weld applied to the peripheral edge of each plate and penetrating the gap between the plates.

FIG. 23 is a schematic illustration of an overview of an SMR reactor, the reactor being disclosed in Example 2.

FIG. 24 is a schematic illustration showing an arrangement of jets providing for the flow of air from an air channel to a fuel channel in the SMR reactor shown in FIG. 23.

FIG. 25 is a schematic illustration showing connections for four product channels used in the reactor shown in FIG. 23.

FIG. 26 is a schematic illustration of the P plate or Plate 1 for the reactor shown in FIG. 23.

FIG. 27 is a schematic illustration of the RP plate or Plate 2 for the reactor illustrated in FIG. 23.

FIG. 28 is a schematic illustration of the Cat plate or Plate 3 for the reactor illustrated in FIG. 23.

FIG. 29 is a schematic illustration of the FA plate or Plate 4 for the reactor illustrated in FIG. 23.

FIG. 30 is a schematic illustration of the AE plate or Plate 5 for the reactor illustrated in FIG. 23.

FIG. 31 is a schematic illustration of the E plate or Plate 6 for the reactor illustrated in FIG. 23.

FIG. 32 is a schematic illustration of the reactor disclosed in Example 2, wherein the reactor includes an exoskeleton for providing structural support.

FIG. 33 is a schematic illustration showing the location of the SMR catalyst and the combustion catalyst in the reactor section of the reactor illustrated in FIG. 23.

FIG. 34 is a schematic illustration of a mask for spray-coating the SMR catalyst used in the reactor illustrated in FIG. 23.

FIG. 35 is a schematic illustration showing redistribution features added to the AE plate for the reactor illustrated in FIG. 23.

FIG. 36 is a plot showing SMR process performance for the reactor disclosed in Example 2.

FIG. 37 is a plot showing combustion performance for the reactor disclosed in Example 2.

FIGS. 38 and 39 are plots showing pressure drops for the reactor disclosed in Example 2.

FIG. 40 is a plot showing load wall temperature profiles for the reactor disclosed in Example 2.

FIG. 41 is a plot showing exhaust gas temperature profiles at the outlet of the reactor disclosed in Example 2.

FIG. 42 is a schematic illustration showing load wall temperature profiles with fuels having varying levels of methane in the fuel for the reactor disclosed in Example 2.

FIG. 43 is a plot showing exhaust temperature variability for the reactor disclosed in Example 2.

FIG. 44 is a plot showing temperature profile along the length of the reactor disclosed in Example 2 as a function of the amount of methane in the combustion fuel.

FIG. 45 is a schematic illustration showing an in-situ coating method for applying a catalyst to the walls of a microchannel reactor.

FIG. 46 is a schematic illustration illustrating an ex-situ coating method for applying a catalyst to the plates of a SMR reactor.

FIG. 47 is an illustration of a masking plate for the R-P plate of a multichannel SMR reactor as described in Example 3.

FIG. 48 is a photograph of a masked plate after coating the plate with a catalyst as described in Example 3.

FIG. 49 consists of a series of photographs of a copper-coated coupon of Inconel 617 from a metal dusting test as discussed in Example 4.

FIG. 50 consists of a series of photographs of an uncoated coupon of Inconel 617 from a metal dusting test as described in Example 4.

FIG. 51 is a SEM of a cross-section of a copper-coated coupon of Inconel 617 after 863 hours of exposure during a metal dusting test as described in Example 4.

FIG. 52 consists of a series of photographs of a TiC/Al₂O₃/Inconel 617 coupon at various stages during a metal dusting test as described in Example 4.

FIG. 53 consists of photographs of three aluminum bronze coated coupons from a metal dusting test as described in Example 4.

FIGS. 54 and 55 show multilayer coatings for providing protection against metal dusting as described in Example 4.

FIG. 56 consists of photographs of a Cat-plate for a SMR reactor before and after a grit blasting procedure for refurbishing the plate as described in Example 5.

FIG. 57 consists of photographs of a R-P plate for a SMR reactor before and after a grit blasting procedure for refurbishing the plate as described in Example 5.

### DETAILED DESCRIPTION

All ranges and ratio limits disclosed in the specification and claims may be combined in any manner. It is to be understood that unless specifically stated otherwise, references to “a,” “an,” and/or “the” may include one or more than one, and that reference to an item in the singular may also include the item in the plural. All combinations specified in the claims may be combined in any manner.

The term “microchannel” refers to a channel having at least one internal dimension of height or width of up to about 10 millimeters (mm), or up to about 5 mm, or up to about 2 mm. The microchannel may have a height, width and length. Both the height and width may be perpendicular to the bulk flow direction of the flow of fluid in the microchannel. The microchannel may comprise at least one inlet and at least one outlet wherein the at least one inlet is distinct from the at least one outlet. The microchannel may not be merely an orifice. The microchannel may not be merely a channel through a zeolite or a mesoporous material. The length of the microchannel may be at least about two times the height or width, or at least about five times the height or width, or at least about ten times the height or width. The height or width may be referred to as the gap between opposed internal walls of the microchannel. The internal height or width of the microchannel may be in the range of about 0.05 to about 10 mm, or from about 0.05 to about 5 mm, or from about 0.05 to about 2 mm, or from about 0.1 to about 2 mm, or from about 0.5 to about 2 mm, is or from about 0.5 to about 1.5 mm, or from about 0.08 to about 1.2 mm. The other internal dimension of height or width may be of any dimension, for example, up to about 10 centimeters (cm), or from about 0.1 to about 10 cm, or from about 0.5 to about 10 cm, or from about 0.5 to about 5 cm. The length of the microchannel may be of any dimension, for example, up to about 250 cm, or from about 5 to about 250 cm, or from about 10 to about 100 cm, or from about 10 to about 75 cm, or from about 10 to about 60 cm. The microchannel may have a cross section having any shape, for example, a square, rectangle, circle, semi-circle, trapezoid, etc. The shape and/or size of the cross section of the microchannel may vary over its length. For example, the height or width may taper from a relatively large dimension to a relatively small dimension, or vice versa, over the length of the microchannel.

The term “process microchannel” refers to a microchannel wherein a process is conducted. The process may comprise any unit operation. The process may comprise a chemical reaction, for example, a steam methane reforming (SMR) reaction. The reactions may include processes for producing ethylene, styrene, formaldehyde, butadiene, and the like. The reaction may comprise a partial oxidation reaction.

The term “microchannel processor” refers to an apparatus comprising one or more process microchannels wherein a process may be conducted. The process may comprise a unit operation wherein one or more fluids are treated. The process may comprise a chemical reaction, such as an SMR reaction.

The term “microchannel reactor” refers to an apparatus comprising one or more process microchannels wherein a reaction process is conducted. The process may comprise any chemical reaction such as an SMR process. When two or more process microchannels are used, the process microchannels may be operated in parallel. The microchannel reactor may include a manifold for providing for the flow of reactants into the one or more process microchannels, and a manifold providing for the flow of product out of the one or more process microchannels. The microchannel reactor may further comprise one or more heat exchange channels adjacent to and/or in thermal contact with the one or more process microchannels. The heat exchange channels may provide heating and/or cooling for the fluids in the process microchannels. The heat exchange channels may be microchannels. The microchannel reactor may include a manifold for providing for the flow of heat is exchange fluid into the heat exchange channels, and a manifold providing for the flow of heat exchange fluid out of the heat exchange channels. The microchannel reactor may also include an exhaust manifold and an exhaust outlet when a combustion reaction is conducted in the heat exchange channels.

The term “welding” refers to a fabrication process that joins materials, usually metals or thermoplastics, by causing coalescence. This may be done by melting the workpieces and/or by adding a filler material to form a pool of molten material (the weld pool) that cools to become a strong joint, with pressure sometimes used in conjunction with heat, or by itself, to produce the weld.

The term “brazing” refers to a metal-joining process whereby a filler material is heated above its melting point and distributed between two or more close-fitting parts by capillary action. The filler metal is brought slightly above its melting temperature while protected by a suitable atmosphere, usually a flux. The filler metal flows over the base metal (known as wetting) and is cooled to join the workpieces together.

The term “diffusion bonding” refers to a process wherein metal parts are held together under an applied force and heated in a vacuum furnace, causing atoms from each part to diffuse to the other. Unlike brazing, no filler alloy is used.

The term contact time refers to the open reactor volume where flow traverses and which contains a reaction catalyst divided by the process inlet stream flowrate calculated at standard conditions. The reactant section contact time refers to the total volume for process flow in a channel within the reactor section of a device which includes the catalyst containing first pass and the accompanying product channel volume that is in thermal contact with the reactant channel and is defined by the same axial locations divided by the total inlet flowrate per channel of process gases calculated at standard conditions. The catalyst channel only contact time refers to the total volume in a channel for process flow in the reactant channel only that contains the process catalyst divided by the total inlet flowrate per channel of process gases calculated at standard conditions. The reactor core contact time refers to the total flow volume per channel of a channel circuit in a reactor that includes a recuperative heat exchange section and a reactor section divided by the total inlet flowrate per channel of process gases calculated at standard conditions.

The term “sufficiently uniform flow” refers to a flow distribution that is not perfect but the amount of flow non-uniformity does not substantially degrade the process performance in that the performance of a devices with more than two channels is within 95% of the performance of a single channel device of equal channel design (length, width, height, and catalyst location).

The term “volume” with respect to volume within a microchannel includes all volume in the microchannel a fluid may flow through or flow by. This volume may include volume within surface features that may be positioned in the microchannel and adapted for the flow of fluid in a flow-through manner or in a flow-by manner.

The term “adjacent” when referring to the position of one channel relative to the position of another channel means directly adjacent such that a wall or walls separate the two channels. The two channels may have a common wall. The common wall may vary in thickness. However, “adjacent” channels may not be separated by an intervening channel that interferes with heat transfer between the channels. One channel may be adjacent to another channel over only part of the channel.

The term “thermal contact” refers to two bodies, for example, two channels, that may or may not be in physical contact with each other or adjacent to each other but still exchange heat with each other. One body in thermal contact with another body may heat or cool the other body.

The term “fluid” refers to a gas, a liquid, a mixture of a gas and a liquid, or a gas or a liquid containing dispersed solids, liquid droplets and/or gaseous bubbles. The droplets and/or bubbles may be irregularly or regularly shaped and may be of similar or different sizes.

The terms “gas” and “vapor” have the same meaning and may be used interchangeably.

The term “residence time” or “average residence time” refers to the internal volume of a space within a channel occupied by a fluid flowing in the space divided by the average volumetric flow rate for the fluid flowing in the space at the average temperature and pressure being used.

The term “surface feature” refers to a depression or a projection in a channel wall and/or internal channel structure that disrupts flow within the channel.

The term “capillary feature” refers to a depression or a projection in a channel wall and/or internal channel structure that does not disrupt flow within the channel when the flow is in the laminar flow regime. For example, a capillary feature may be a depression in a wall that is substantially perpendicular to the flow direction. Capillary features may be cross hatched or have other non-regular shapes such as those produced by surface roughening. In general, flow may be substantially stagnant in a capillary feature and this stagnant flow region may enable an enhanced reaction rate by creating a safe harbor for reactants to continue to contact the catalyst before diffusing back into fast moving flow stream adjacent to the capillary features.

The term “bulk flow direction” refers to the vector through which fluid may travel in an open path in a channel.

The term “bulk flow region” refers to open areas within a channel (e.g., a process microchannel). A contiguous bulk flow region may allow rapid fluid flow through a channel without significant pressure drop. In one embodiment, the flow in the bulk flow region may be laminar. A bulk flow region may comprise at least about 5% of the internal volume and/or cross-sectional area of a microchannel, and in one embodiment from about 5% to about 100%, and in one embodiment from about 5% to about 99%, and in one embodiment about 5% to about 95%, and in one embodiment from about 5% to about 90%, and in one embodiment from about 30% to about 80% of the internal volume and/or cross-sectional area of the microchannel.

The term “cross-sectional area” of a channel (e.g., process microchannel) refers to an area measured perpendicular to the direction of the bulk flow of fluid in the channel and may include all areas within the channel including any surface features that may be present, but does not include the channel walls. For channels that curve along their length, the cross-sectional area may be measured perpendicular to the direction of bulk flow at a selected point along a line that parallels the length and is at the center (by area) of the channel. Dimensions of height and width may be measured from one interior channel wall to the opposite interior channel wall. These dimensions may be average values that account for variations caused by surface features, surface roughness, and the like.

The term “process fluid” refers to reactants, product, diluent and/or other fluid that enters, flows in and/or flows out of a process microchannel.

The term “reactants” refers to reactants used in a chemical reaction. For an

SMR reaction, the reactants may comprise steam and methane. For a combustion reaction, the reactants may comprise a fuel (e.g., hydrogen, hydrocarbon such as methane, etc.) and an oxygen source such as air.

The term “reaction zone” refers to the space within a microchannel wherein a chemical reaction occurs or wherein a chemical conversion of at least one species occurs. The reaction zone may contain one or more catalysts.

The term “heat exchange channel” refers to a channel having a heat exchange fluid in it that gives off heat and/or absorbs heat. The heat exchange channel may absorb heat from or give off heat to an adjacent channel (e.g., process microchannel) and/or one or more channels in thermal contact with the heat exchange channel. The heat exchange channel may absorb heat from or give off heat to channels that are adjacent to each other but not adjacent to the heat exchange channel. In one embodiment, one, two, three or more channels may be adjacent to each other and positioned between two heat exchange channels.

The term “heat transfer wall” refers to a common wall between a process microchannel and an adjacent heat exchange channel where heat transfers from one channel to the other through the common wall.

The term “heat exchange fluid” refers to a fluid that may give off heat and/or absorb heat.

The term “conversion of reactant” refers to the reactant mole change between a fluid flowing into a microchannel reactor and a fluid flowing out of the microchannel reactor divided by the moles of reactant in the fluid flowing into the microchannel reactor.

The term “mm” may refer to millimeter. The term “nm” may refer to nanometer. The term “ms” may refer to millisecond. The term “μs” may refer to microsecond. The term “μm” may refer to micron or micrometer. The terms “micron” and “micrometer” have the same meaning and may be used interchangeably. The term m/s may refer to meters per second. The term “kg” refers to kilograms. Unless otherwise indicated, all pressures are expressed in terms of absolute pressure.

The inventive apparatus may comprise one or more process layers, and one or more heat exchange layers. The apparatus may be used for conducting any unit operation. The unit operation may be conducted in the process layer of the apparatus, and heating or cooling may be provided by the heat exchange layer. When more than one process layer and more than one heat exchange layer are used, they may be aligned in alternating sequence, or two or more process layers and/or two or more heat exchange layers may be positioned adjacent to each other.

The unit operation that may be conducted in the one or more process layers may comprise a chemical reaction, vaporization, compression, chemical separation, distillation, condensation, mixing, heating, cooling, or a combination of two or more thereof.

The chemical reaction may comprise any chemical reaction. The chemical reaction may comprise a methanol synthesis reaction, dimethyl ether synthesis reaction, ammonia synthesis reaction, water gas shift reaction, acetylation addition reaction, alkylation, dealkylation, hydrodealkylation, reductive alkylation, amination, aromatization, arylation, autothermal reforming, carbonylation, decarbonylation, reductive carbonylation, carboxylation, reductive carboxylation, reductive coupling, condensation, cracking, hydrocracking, cyclization, cyclooligomerization, dehalogenation, dimerization, epoxidation, esterification, Fischer-Tropsch reaction, halogenation, hydrohalogenation, homologation, hydration, dehydration, hydrogenation, dehydrogenation, hydrocarboxylation, hydroformylation, hydrogenolysis, hydrometallation, hydrosilation, hydrolysis, hydrotreating, isomerization, methylation, demethylation, metathesis, nitration, oxidation, partial oxidation, polymerization, reduction, reformation, reverse water gas shift, sulfonation, telomerization, transesterification, trimerization, Sabatier reaction, carbon dioxide reforming, partial oxidation, preferential oxidation, or preferential methanation reaction. The chemical reaction may comprise an SMR reaction. The chemical reactions may include those for producing ethylene, styrene, formaldehyde, butadiene, and the like.

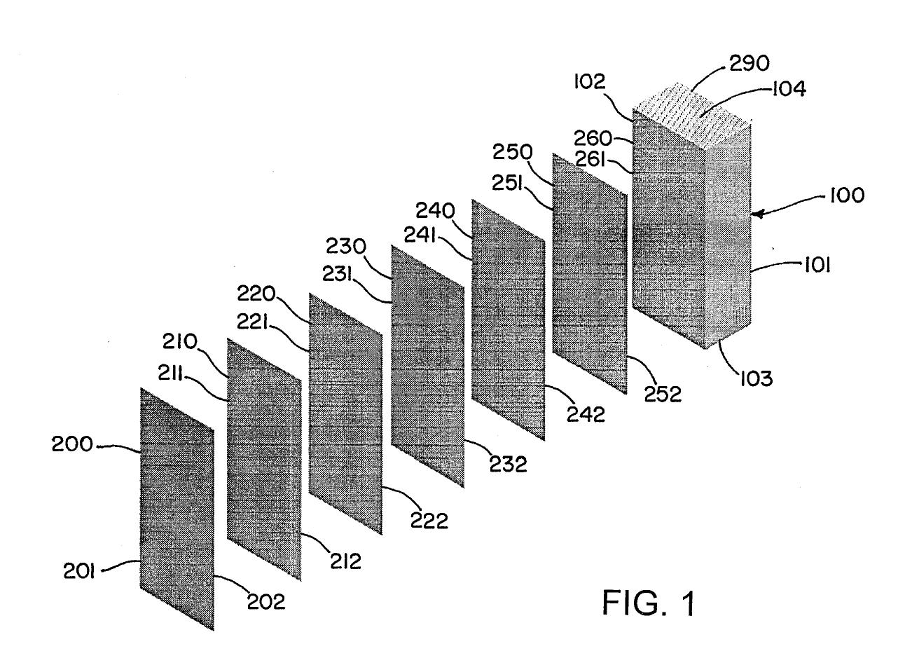

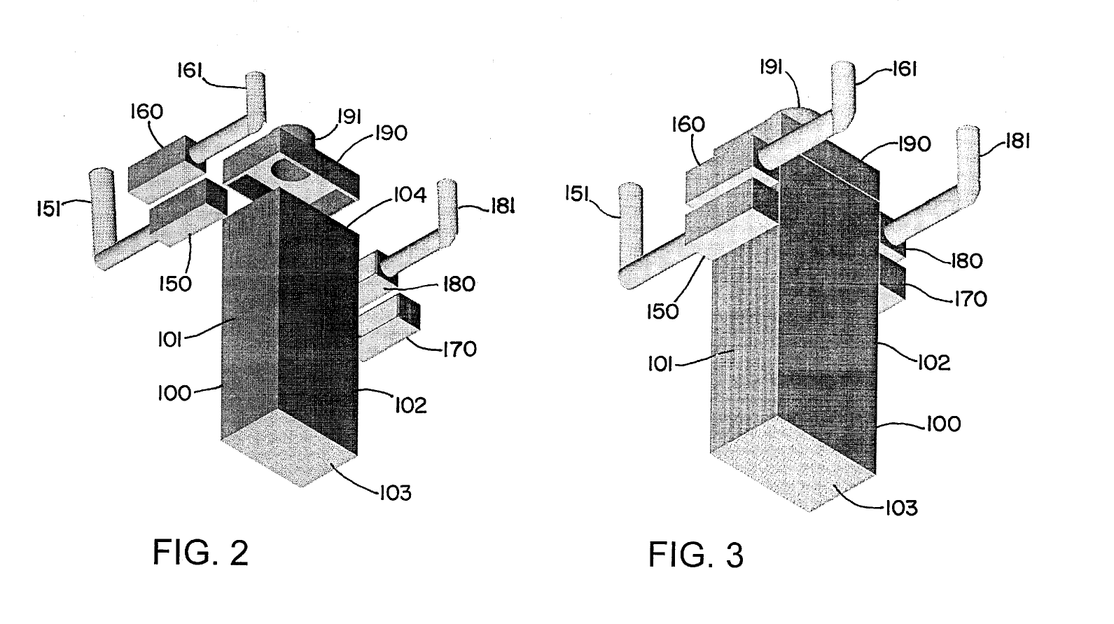

Referring to the drawings, and initially to FIGS. 1 to 4, the inventive apparatus may comprise a stack of plates 100. The stack 100 may be used as the core assembly for a microchannel processor. The stack 100 may comprise one or more process layers and one or more heat exchange layers positioned adjacent one another or in thermal contact with one another. The stack 100 may comprise, for example, from 1 to about 1,000, or from 1 to about 500, or from 1 to about 200, or is from 1 to about 100, or from 1 to about 50, or from 1 to about 30, or from 1 to about 20, process layers and corresponding heat exchange layers adjacent to or in thermal contact with the process layers. The stack 100 may include sides 101, 102, 103 and 104 formed by the peripheral edges of the plates. The peripheral edge of each plate on each of the sides 101, 102, 103 and 104 may be welded to the peripheral edge of the next adjacent plate. In this way, the stack 100 may comprise a perimeter seal on each of the sides 101, 102, 103 and 104 formed by the welds. The welds may also be used to provide structural integrity for the stack 100.

The stack 100 may be oriented with the plates aligned vertically and positioned side-by-side to facilitate flow of the process and heat exchange fluids. Alternatively, the stack 100 may be aligned in such a manner to provide for the plates being oriented horizontally, or at an angle to the horizontal. The stack 100 may have welded to its sides manifolds 150, 160, 170 and 180. These manifolds may be used to provide for the flow of reactants into the stack 100, product out of the stack 100, and heat exchange fluid into and out of the stack. Two of the manifolds may be used to provide for the flow of fuel and air into the stack 100 when a combustion reaction is conducted in the heat exchange layer. Also, exhaust outlet 190 may be welded to the top of the stack 100 for removing exhaust when a combustion reaction is conducted in the heat exchange layer.

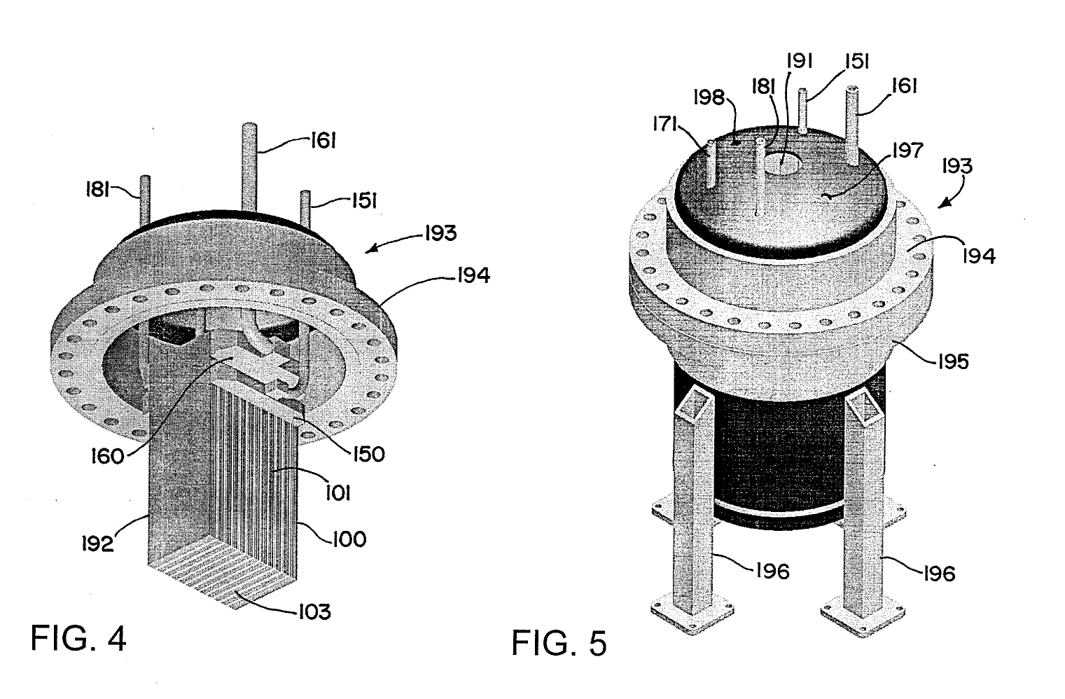

The stack 100, with the manifolds 150, 160, 170 and 180 welded to is sides, and exhaust outlet 190 welded to its top end, may be referred to as microchannel processor 192. Referring to FIGS. 4 and 5, microchannel processor 192 may be positioned in containment vessel 193. The containment vessel 193 may include top head 194, containment section 195, support legs 196, containment gas inlet 197, temperature control port 198, and a drain port (not shown in the drawings) at the bottom of the containment section 195. Inlet and outlet pipes 151, 161, 171 and 181 extend from corresponding manifolds 150, 160, 170 and 180, and project through the top head 194. Similarly, exhaust outlet opening 191 extends from exhaust outlet 190 through the top head 194. The containment vessel 193 may include appropriate insulation within its interior and/or on its exterior surface, and may be constructed using any material that can provide structural integrity for the desired end use. These materials may include: steel (e.g., stainless steel, carbon steel, and the like); aluminum; titanium; nickel; platinum; rhodium; copper; chromium; alloys containing is any of the foregoing metals; monel; inconel; brass; polymers (e.g., thermoset resins); ceramics; glass; composites comprising one or more polymers (e.g., thermoset resins) and fiberglass; quartz; silicon; or a combination of two or more thereof. The containment vessel may be constructed of carbon steel and rated to 450 psig (3.10 MPa) at 260° C. The outside diameter (OD) of the containment vessel 193 may be of any desired dimension for the intended use. For example, for an SMR reactor, the OD of the containment vessel may be about 30 inches (76.2 cm), or about 32 inches (81.3 cm), or about 36 inches (91.4 cm). The height of the containment vessel may be from about 24 to about 200 inches (about 61 to about 508 cm), or from about 48 to about 72 inches (about 122 to about 183 cm), or about 60 inches (about 152 cm).

The containment vessel may include a control mechanism to maintain the pressure within the containment vessel at a level at least as high as the internal pressure within the stack. The control mechanism for maintaining pressure within the containment vessel may comprise a check valve and/or a pressure regulator. The check valve or regulator may be programmed to activate at any desired internal pressure for the containment vessel, for example, about 400 psig (2.76 MPa). Either or both of these may be used in combination with a system of pipes, valves, controllers, and the like, to ensure that the pressure in the containment vessel is maintained at a level that is at least as high as the internal pressure within the stack. This is done in part to protect the peripheral welds used to seal the stack. A significant decrease in the pressure within the containment vessel without a corresponding decrease of the internal pressure within the stack could result in a costly rupture of the peripheral welds. The control mechanism may be designed to allow for diversion of one or more process gases into the containment vessel in the event the pressure exerted by the containment gas decreases.

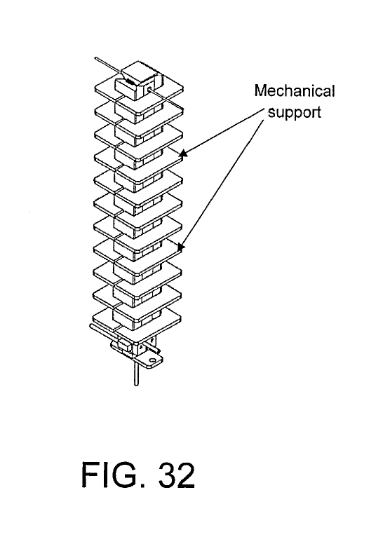

In an alternate embodiment, an exoskeleton may be used to provide structural support for the stack 100. This is shown in FIG. 32. The exoskeleton may comprise an array of stiffening members which are held in intimate contact with major exterior faces of the endplates of the stack. The stiffness of the members of the array may be such that they resist bending in the stacking direction (i.e. the direction orthogonal to the plane of the stack). The exoskeleton may be welded to the stack. Alternatively, the exoskeleton may be attached to the stack by brazing, gluing, or other means.

With an exoskeleton, welded reinforcement members may have a rectangular cross sections oriented with the longer side parallel to the direction of load application to provide increased stiffness to resist bending stress. This may permit the use of thinner plates and reduce the weight and cost of material required to support equal loads.

Exoskeletons may be superior to clamps. Clamps may be more easily removed than exoskeletons, especially if bolted in place or made with a quick release mechanism. Exoskeletons typically require cutting or grinding for removal. Clamps having thick plates with threaded fasteners may be used. However, the plates for these clamps would need to be strong enough for the bending stress since the threaded fasteners would not be loaded in this direction. The threaded fasteners would need to be strong enough for the full tension stress caused by the force created by the pressure acting on the plates. On the other hand, the exoskeleton provides additional support to the plates in both cases.

The stack 100 may comprise one or plurality of repeat units, wherein each repeat unit is the same and each comprises one or more process layers and one or more heat exchange layers. For example, a repeat unit may comprise from 1 to about 100, or from 1 to about 20, or from 1 to about 10, or from 1 to about 5, or from 1 to about 3, or about 2 process layers; and from 1 to about 100, or from 1 to about 20, or from 1 to about 10, or from 1 to about 5, or from 1 to about 3, or about 2 heat exchange layers. The repeat units may be aligned horizontally and stacked one above another, aligned vertically and positioned side-by-side, or they may be aligned at an angle to the horizontal. Within each repeat unit the process layers and heat exchange layers may be aligned in alternating sequence with a process layer adjacent to a heat exchange layer, which in turn is adjacent to another process layer, which in turn is adjacent to another heat exchange layer, etc. Alternatively, two or more process layers and/or two or more heat exchange layers may be iii positioned adjacent to one another.

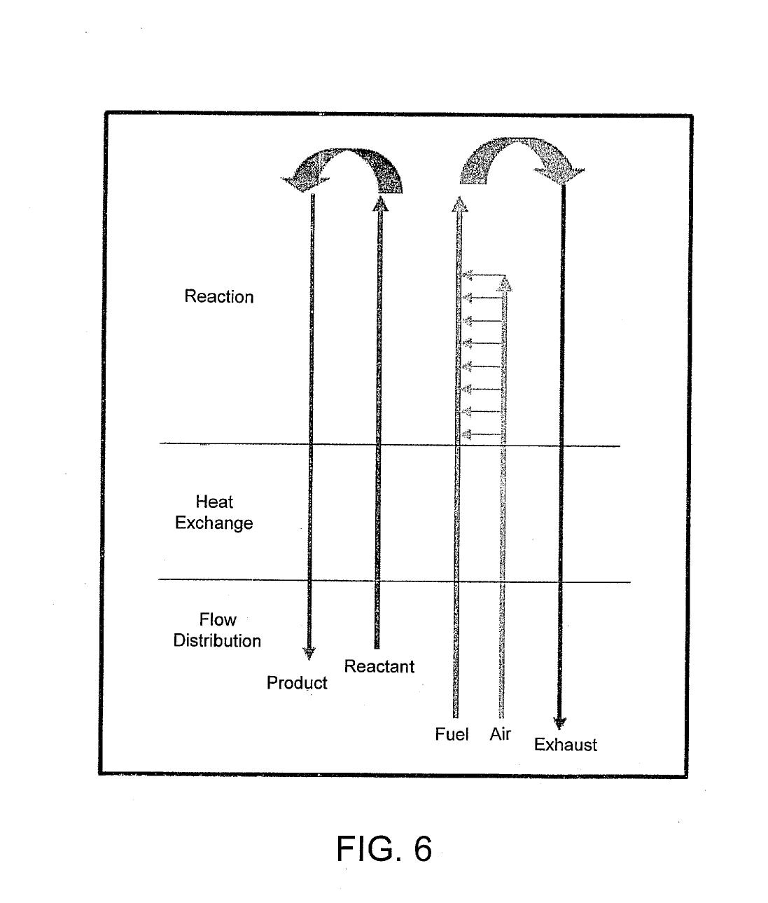

Referring to FIG. 6, when the stack 100 is adapted for use in conducting an SMR reaction, the process layer may comprise a reactant layer, a product layer, and a process u-turn positioned at an end of the reactant layer and product layer to allow for the flow of fluid from the reactant layer to the product layer. The reactant layer is may be positioned adjacent to the product layer. In the process layer, the reactants may contact the catalyst and react to form the product, with the product then flowing out of the product layer. The heat exchange layer may comprise a fuel layer, an air layer positioned adjacent to the fuel layer, a heat exchange wall positioned between the fuel layer and the air layer, a plurality of openings or jets in the heat exchange wall to allow for the flow of air from the air layer into the fuel layer, a combustion catalyst positioned in the fuel layer, an exhaust layer, and a heat exchange u-turn positioned at an end of the fuel layer and an end of the exhaust layer to allow for the flow of exhaust from the fuel layer to the exhaust layer.

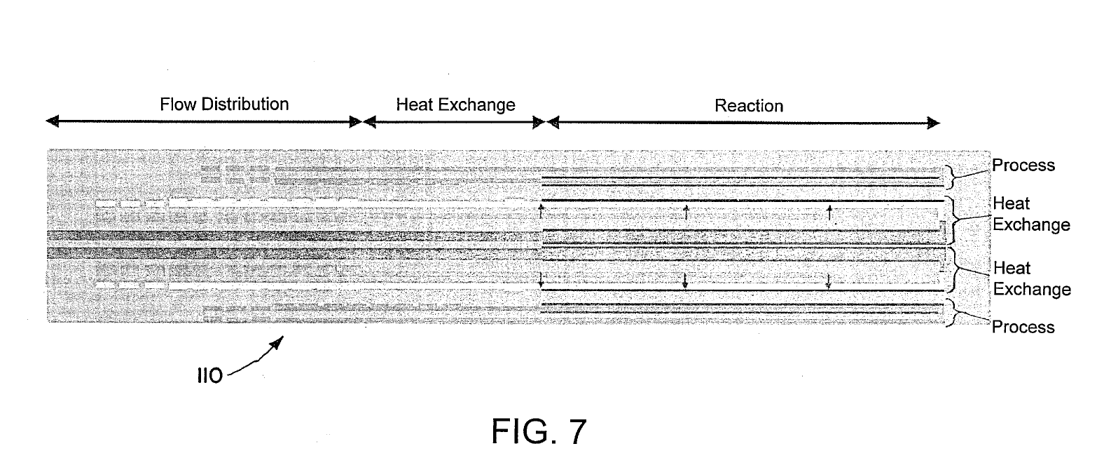

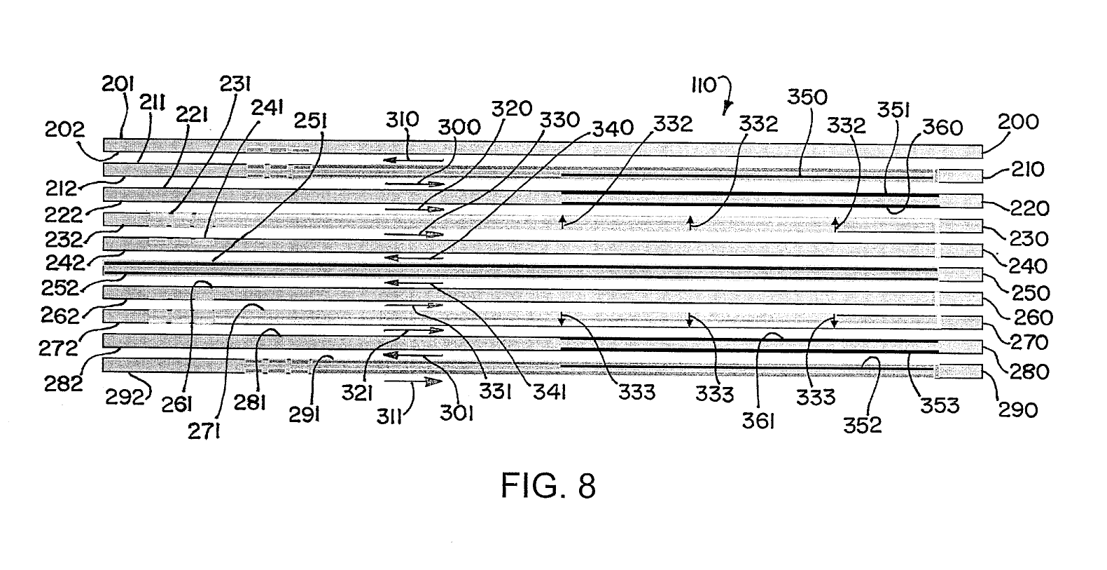

When the stack 100 is adapted for use as an SMR reactor, the repeat unit 110 shown in FIGS. 7 and 8 may be used to construct the stack. As shown in FIG. 7, repeat unit 110 contains two heat exchange layers positioned adjacent to each other, and an SMR process layer positioned on each side of the heat exchange layers. The repeat unit 110 contains 10 plates which are shown in FIG. 8 as being separated from each other for purposes of illustration, but in actual use the plates would be in contact with each other. The peripheral edge of each plate may be welded to the peripheral edge of the next adjacent plate to provide a peripheral seal for the stack. The repeat unit 110 contains plates 200, 210, 220, 230, 240, 250, 260, 270, 280 and 290. Each side of each plate may contain microchannels, internal manifolds, capillary features and/or surface features formed on its surface; and each plate may contain air openings or jets, and/or u-turn or openings or slots projecting through the plate to provide for the functioning of two SMR process layers and two combustion layers. Each of the plates may be fabricated using known techniques including wire electrodischarge machining, conventional machining, laser cutting, photochemical machining, electrochemical machining, stamping, etching (for example, chemical, photochemical or plasma etching) and combinations thereof.

In the following discussion relative to the alignment of the plates 200, 210, 220, 230, 240, 250, 260, 270, 280 and 290, reference is made to the top surface and bottom surface of each plate as depicted in FIG. 8, although as indicated above, when positioned in the stack 100 and used for an SMR reaction, the plates 200, 210, 220, 230, 240, 250, 260, 270, 280 and 290 may be vertically aligned, not horizontally aligned as shown in FIG. 8.

Referring to FIG. 8, plate 200 has a top surface 201 and a bottom surface 202. Plate 210 has a top surface 211 and a bottom surface 212. Plate 220 has a top surface 221 and a bottom surface 222. Plate 230 has a top surface 231 and a bottom surface 232. Plate 240 has a top surface 241 and a bottom surface 242. Plate 250 has a top surface 251 and a bottom surface 252. Plate 260 has a top surface 261 and a bottom surface 262. Plate 270 has a top surface 271 and a bottom surface 272. Plate 280 has a top surface 281 and a bottom surface 282.

Plate 290 has a top surface 291 and a bottom surface 292. In operation, product from the SMR reaction flows from right to left (as illustrated in FIG. 8) as shown by arrows 310 and 311. The reactants for the SMR process flow from left to right as shown by arrows 300 and 301. Fuel flows from left to right in the direction indicated by arrows 320 and 321. Air flows from left to right in the direction indicated by arrows 330 and 331. In each case, the wall separating the air layer and heat exchange layer contains openings or jets 332 or 333 to allow the air to flow from the air layer into the fuel layer, combine with the fuel to form a fuel-air mixture, and then undergo combustion. Exhaust from the combustion reaction flows from right to left as indicated by arrows 340 and 341. SMR catalyst layers 350, 351, 352 and 353 are provided for catalyzing the SMR reactions. Combustion catalyst layers 360 and 361 are provided for catalyzing the combustion reactions.

The plates 200, 210, 220, 230, 240, 250, 260, 270, 280 and 290 may have a common length and width in order to provide the repeat unit 110 with even or planar sides as well as even or planar tops and bottoms. The lengths of each plate may be, for example, in the range from about 30 to about 250 centimeters, or from about 45 to about 150 centimeters, or about 29 inches (73.66 cm). The width of each of the plates may be in the range from about 15 to about 90 cm, or from about 20 to about 40 cm, or about 10.74 inches (27.28 cm). The height or thickness of each plate can be the same or different, but for facilitated manufacturing purposes, it is advantageous for each of the plates to have the same height or thickness. The height or thickness of each of the plates may range from about 0.8 to about 25 mm, or from about 1.5 to about 10 mm, or about 0.125 inch (3.175 mm). The overall height of the repeat unit 110 may be from about 0.1 to about 5 inches (about 0.254 to about 12.7 cm), or from about 0.5 to about 3 inches (about 1.27 to about 7.62 cm), or from about 0.75 to about 2.5 inches (about 1.91 to about 6.35 cm), or from is about 1 to about 1.5 inches (about 2.54 to about 3.81 cm), or about 1.25 inches (3.175 cm). The overall height of the stack 100 may be from about 1 to about 50 inches (about 2.54 to about 127 cm), or from about 3 to about 24 inches (about 7.62 to about 60.96 cm), or from about 7 to about 15 inches (about 17.78 to about 38.1 cm), or about 10.125 inches (25.72 cm). With one exception, each of the plates 200, 210, 220, 230, 240, 250, 260, 270, 280 and 290 has microchannels, internal manifolds, capillary features, and/or surface features formed on the plate surfaces, and/or openings or jets, or u-turn openings or slots projecting through the plates to provide for the flow of reactants, product, fuel, air and exhaust. The one exception is the top 201 of plate 200 which is blank due to the fact that plate 200 may be used as an end plate for the stack 100. In the discussion that follows, the use terms “air,” “air layer,” “air channel,” and the like, are used to refer to air as a component in the combustion reaction conducted in the combustion layer. However, as indicated below, the combustion reaction may employ, as an alternative to air, oxygen sources such as pure oxygen, oxygen enriched air or gaseous mixture comprising oxygen and an inert gas. Thus, when an air layer, air channel, and the like, are referenced in terms of the structure of the inventive apparatus, it is to be understood that any of the foregoing alternatives may be substituted for the air.

The depth of each microchannel may be in the range of about 0.05 to about 10 mm, or from about 0.05 to about 5 mm, or from about 0.05 to about 2 mm, or from about 0.1 to about 2 mm, or from about 0.5 to about 2 mm, or from about 0.5 to about 1.5 mm, or from about 0.08 to about 1.2 mm. The width of each microchannel may be up to about 10 cm, or from about 0.1 to about 10 cm, or from about 0.5 to about 10 cm, or from about 0.5 to about 5 cm.

The internal manifolds may be used to provide for a uniform distribution of mass flow into or out of the microchannels. Each internal manifold may be used to provide for the flow of fluid into or out of from about 2 to about 1000 microchannels, or from 2 to about 100 microchannels, or from about 2 to about 50 microchannels, or from about 2 to about 10, or from 2 to about 6, or about 4 microchannels. The depth of each manifold may correspond to the depth of the microchannels connected to the manifold. The width of each manifold may correspond to the combined widths of the microchannels connected to manifold, or from about 1 to about 99 percent, or is from about 1 to about 90 percent, of the combined widths to provide for desired flow resistance into or out of the microchannels. The uniformity of the mass flow distribution between the microchannels may be defined by the Quality Index Factor (Q-factor) indicated below. A Q-factor of 0% means absolute uniform distribution.

$$Q = \frac{{\dot{m}}_{max} - {\dot{m}}_{min}}{{\dot{m}}_{max}} \times 00$$

In the above formula “m” refers to mass flow. A change in the cross-sectional area may result in a difference in shear stress on the wall. In one embodiment, the Q-factor for the inventive microchannel processor may be less than about 50%, or less than about 20%, or less than about 5%, or less than about 1%.

The surface features and/or capillary features may comprise depressions in and/or projections from one or more of the plate surfaces. The surface features may be in the form of circles, spheres, hemispheres, frustrums, oblongs, squares, rectangles, angled rectangles, checks, chevrons, vanes, air foils, wavy shapes, and the like. Combinations of two or more of the foregoing may be used. The surface features may contain subfeatures where the major walls of the surface features further contain smaller surface features that may take the form of notches, waves, indents, holes, burrs, checks, scallops, and the like. The surface features may be referred to as passive surface features or passive mixing features. The surface features may be used to disrupt flow (for example, disrupt laminar flow streamlines) and create advective flow at an angle to the bulk flow direction. The depth or height of each surface feature may be in the range of about 0.05 to about 5 mm, or from about 0.1 to about 5 mm, or from about 0.1 to about 3 mm, or from about 0.1 to about 2 mm, or from about 0.4 to about 2 mm, or from about 0.5 to about 1.5 mm, or from about 0.08 to about 1.2 mm.

In the heat exchange layers, the plates separating the air channels from the fuel channels may include openings or jets 332 or 333 to allow for the flow of air from the air channels into the fuel channels. These openings or jets may have average diameters in the range from about 0.1 to about 10 mm, or from about 0.1 to about 5 mm, or from about 0.1 to about 2.5 mm, or from about 0.25 to about 1.25 mm, or from about 0.25 to about 0.75 mm, or about 0.015 inch (0.381 mm). Multiple openings or jets, for example, from about 2 to about 5, or from 2 to about 4, or about 3, openings or jets may be provided in parallel at each location to control flow is distribution and prevent diffusion of flame into the air channels. Alternatively, the jets may be offset axially or laterally along the length of the reaction channel. The number of openings or jets that may be used may be in the range from about 0.1 to about 12 openings or jets per cm², or from about 0.1 to about 5 openings or jets per cm²

A number of the plates include u-turn openings or slots to allow for the flow of fluid from one plate surface to another. The gap or width of each u-turn opening or slot may be in the range from about 0.25 to about 5 mm, or from about 0.5 to about 2.5 mm, or about 0.04 inch (1.02 mm).

Each plate has a peripheral edge on each of its sides, and a border adjacent each peripheral edge. Each border may have a thickness in the range from about 1 to about 100 mm, or from about 1 to about 75 mm, or from about 5 to about 50 mm, or from about 10 to about 30 mm.

The plates 200, 210, 220, 230, 240, 250, 260, 270, 280 and 290 may be constructed of any metal or metal alloy having the required properties for structural integrity to operate at the temperatures and pressures intended for the desired end use. The metals and metal alloys may include: steel (e.g., stainless steel, carbon steel, and the like); aluminum; titanium; nickel; platinum; rhodium; copper; chromium; alloys containing any of the foregoing metals; monel; inconel; brass; or a combination of two or more thereof. Inconel 617, which is described below, may be used.

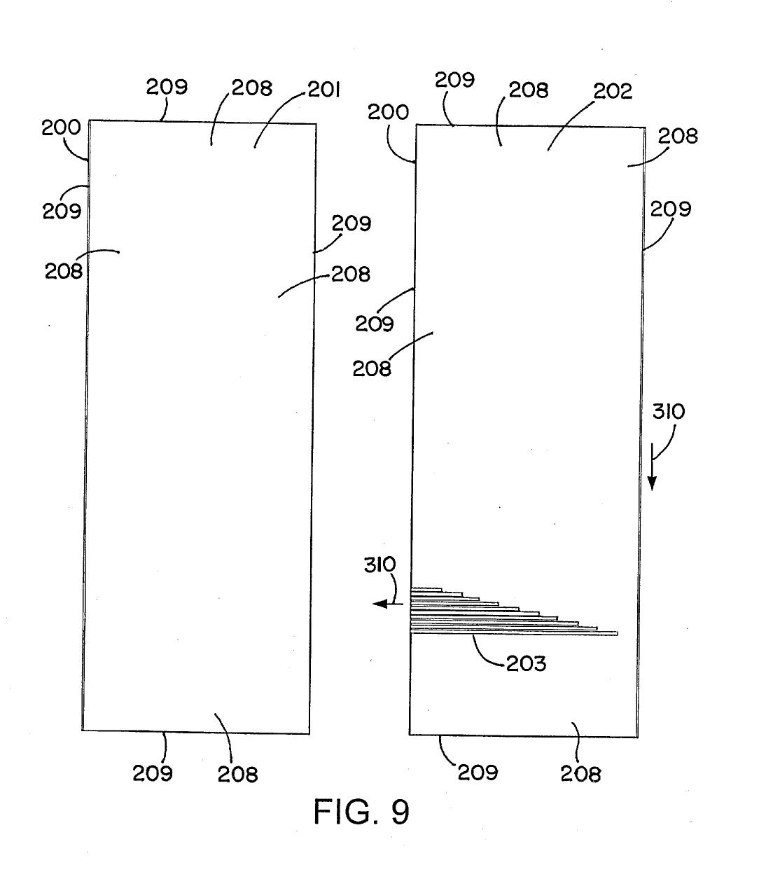

The top and bottom of each of plates 200, 210, 220, 230, 240, 250, 260, 270, 280 and 290 are illustrated in FIGS. 9-18, respectively. Referring to FIG. 9, plate 200 has top surface 201 which is blank due to the fact that this surface may be used as the exterior surface of an end plate for the stack 100. The bottom surface 202 includes internal manifold 203 which may be used to provide for the flow of product from the SMR reaction out of the stack 100 as indicated by arrows 310. Each side of the plate 200, that is plate surfaces 201 and 202, has a border 208. The plate 200 includes a peripheral edge 209 on each of the four sides of the plate. In the formation of the stack 100, or of the repeat unit 110, each of the peripheral edges 209 has a welding material applied to it. When the welding material is applied, it will typically penetrate beyond the peripheral edge 209 in contact with a portion of the border 208 on at least the surface 202 of plate 200. During refurbishing, the welding is material may be removed, for example, by milling, grinding and/or cutting, from the peripheral edges 209 and as a result part of the border 208 may also be removed.

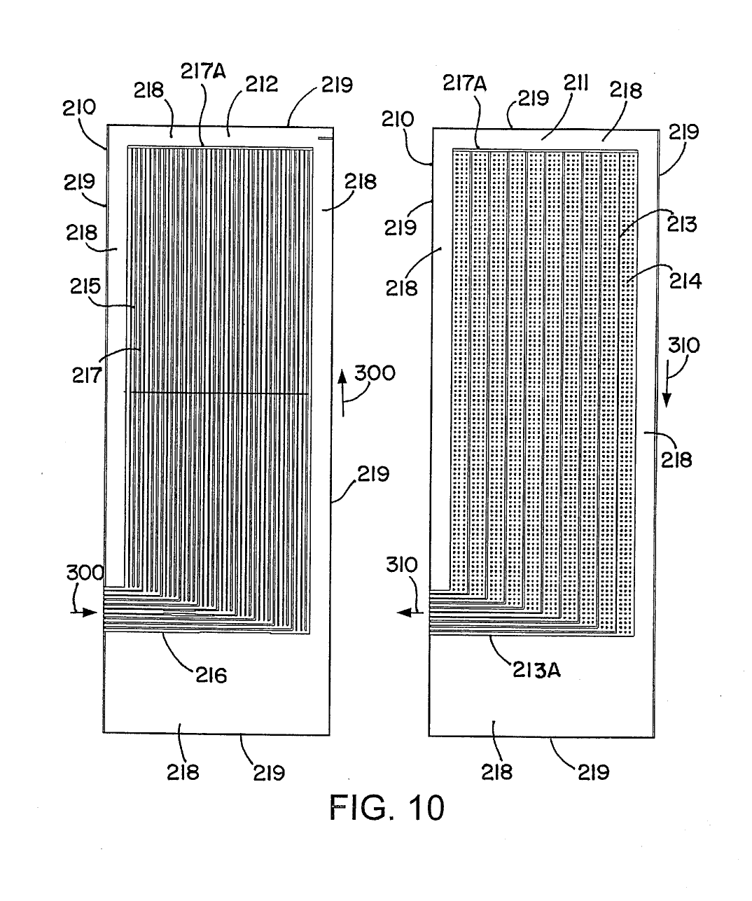

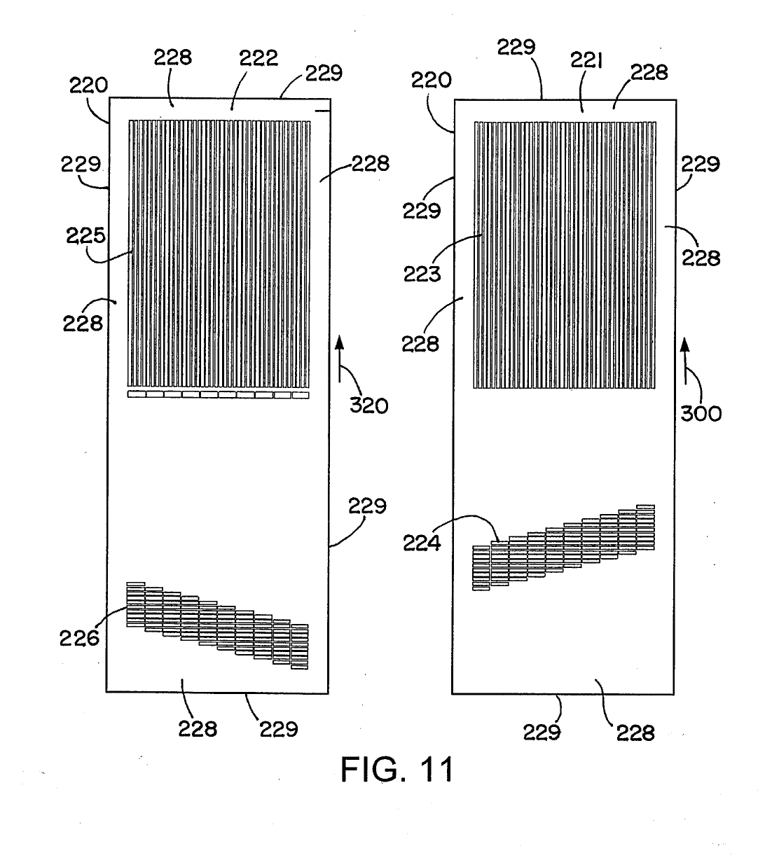

Plate 210 is illustrated in FIG. 10. The top surface 211 includes microchannels 213 and internal manifold 213A which may be used to provide for the flow of product from the SMR reaction in the direction indicated by arrow 310. The microchannels 213 include surface features 214 which may be used to disrupt the flow of product flowing through the process microchannels 213. The bottom surface 212 includes microchannels 215 and internal manifold 216 which may be used to provide for the flow of the SMR reactants in the direction indicated by arrows 300. The microchannels 215 include reaction zone 217 wherein a catalyst for the SMR reaction is coated on the microchannels. The reactants, which may comprise a mixture of methane and steam, flow through the reaction zone 217, contact the catalyst and react to form product. The product may comprise a mixture of carbon monoxide and hydrogen. The plate 210 includes u-turn opening 217A to provide for the flow of product from the process microchannels 215 to the process microchannels 213. Each side of the plate 210, that is plate surfaces 211 and 212, has a border 218. The plate 210 includes a peripheral edge 219 on each of the four sides of the plate. In the formation of the stack 100, or of the repeat unit 110, each of the peripheral edges 219 has a welding material applied to it. When the welding material is applied, it will typically penetrate beyond the peripheral edge 219 in contact with a portion of the border 218 on each side of the plate 210. During refurbishing, the welding material may be removed, for example, by milling, grinding and/or cutting, from the peripheral edges 219 and as a result part of the border 218 may also be removed.

Plate 220 is illustrated in FIG. 11. The top surface 221 includes process microchannels 223, which are coated with an SMR catalyst, and surface features 224 for redistributing flow of the SMR reactants and/or retaining coated catalyst in the channels. The bottom surface 222 includes microchannels 225, which are coated with a combustion catalyst, and surface features or capillary surface features 226 for redistributing flow of the fuel and/or retaining coated catalyst in the channels. Each side of the plate 220, that is plate surfaces 221 and 222, has a border 228. The plate 220 includes a peripheral edge 229 on each of the four sides of the plate. In the formation of the stack 100, or of the repeat unit 110, each of the peripheral edges 229 has a welding material applied to it. When the welding material is applied, it will typically penetrate beyond the peripheral edge 229 in contact with a portion of the border 228 on each side of the plate 220. During refurbishing, the welding material may be removed, for example, by milling, grinding and/or cutting, from the peripheral edges 229 and as a result part of the border 228 may also be removed.

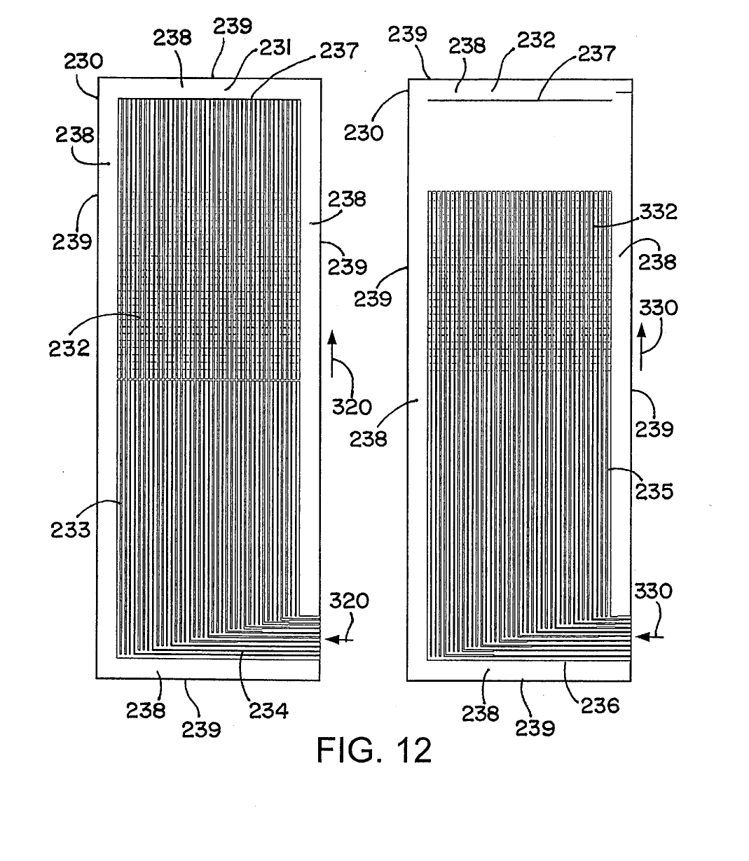

Plate 230 is illustrated in FIG. 12. The top surface 231 includes microchannels 233 and internal manifold 234 which are used to provide for the flow of fuel in the direction indicated by arrows 320. The bottom surface 232 includes microchannels 235 and internal manifold 236 which are used to provide for the flow of air in the direction indicated by arrows 330. The plate includes openings or jets 332 to provide for the flow of air from the microchannels 235 through the plate into the microchannels 233 where it may combine with the fuel to form a fuel-air mixture. The plate 230 includes opening or slot 237 to provide a u-turn for the flow of exhaust from the microchannels 233. Each side of the plate 230, that is plate surfaces 231 and 232, has a border 238. The plate 230 includes a peripheral edge 239 on each of the four sides of the plate. In the formation of the stack 100, or of the repeat unit 110, each of the peripheral edges 239 has a welding material applied to it. When the welding material is applied, it will typically penetrate beyond the peripheral edge 239 in contact with a portion of the border 238 on each side of the plate 210. During refurbishing, the welding material may be removed, for example, by milling, grinding and/or cutting, from the peripheral edges 239 and as a result part of the border 238 may also be removed.

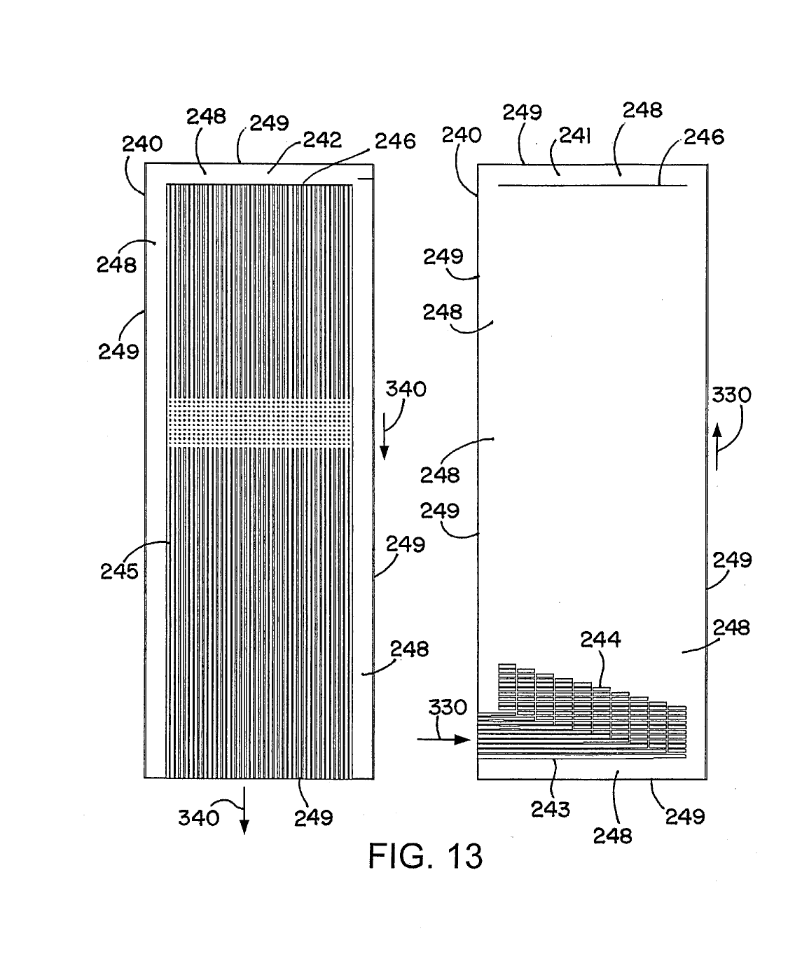

Plate 240 is illustrated in FIG. 13. The top surface 241 includes internal manifold 243 which is used to provide for the flow of air in the direction indicated by arrow 330. The top surface 241 may also include surface features 244 to provide for redistribution of the flow of the air. The bottom surface 242 includes microchannels 245 which are used to provide for the flow of exhaust in the direction indicated by arrows 340. The plate 240 includes opening or slot 246 to provide a u-turn for the flow of exhaust from the microchannels 233 of plate 230 to microchannels 253 of plate 250. Each side of the plate 240, that is plate surfaces 241 and 242, has a border 248. The plate 240 includes a peripheral edge 249 on each of the four sides of the plate. In the formation of the stack 100, or of the repeat unit 110, each of the is peripheral edges 249 has a welding material applied to it. When the welding material is applied, it will typically penetrate beyond the peripheral edge 249 in contact with a portion of the border 248 on each side of the plate 240. During refurbishing, the welding material may be removed, for example, by milling, grinding and/or cutting, from the peripheral edges 249 and as a result part of the border 248 may also be removed.

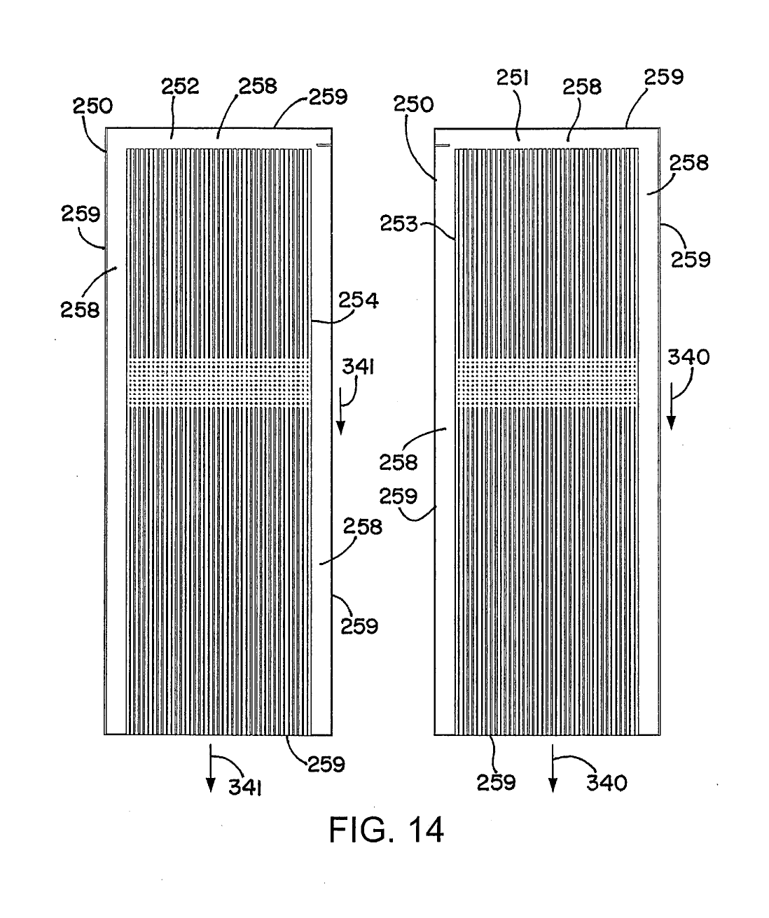

Plate 250 is illustrated in FIG. 14. The top surface 251 includes microchannels 253 which are used to provide for the flow of exhaust in the direction indicated by arrow 340. The bottom surface 252 includes microchannels 254 which are used to provide for the flow of exhaust in the direction indicated by arrows 341. Each side of the plate 250, that is plate surfaces 251 and 252, has a border 258. The plate 250 includes a peripheral edge 259 on each of the four sides of the plate. In the formation of the stack 100, or of the repeat unit 110, each of the peripheral edges 259 has a welding material applied to it. When the welding material is applied, it will typically penetrate beyond the peripheral edge 259 in contact with a portion of the border 258 on each side of the plate 250. During refurbishing, the welding material may be removed, for example, by milling, grinding and/or cutting, from the peripheral edges 259 and as a result part of the border 258 may also be removed.

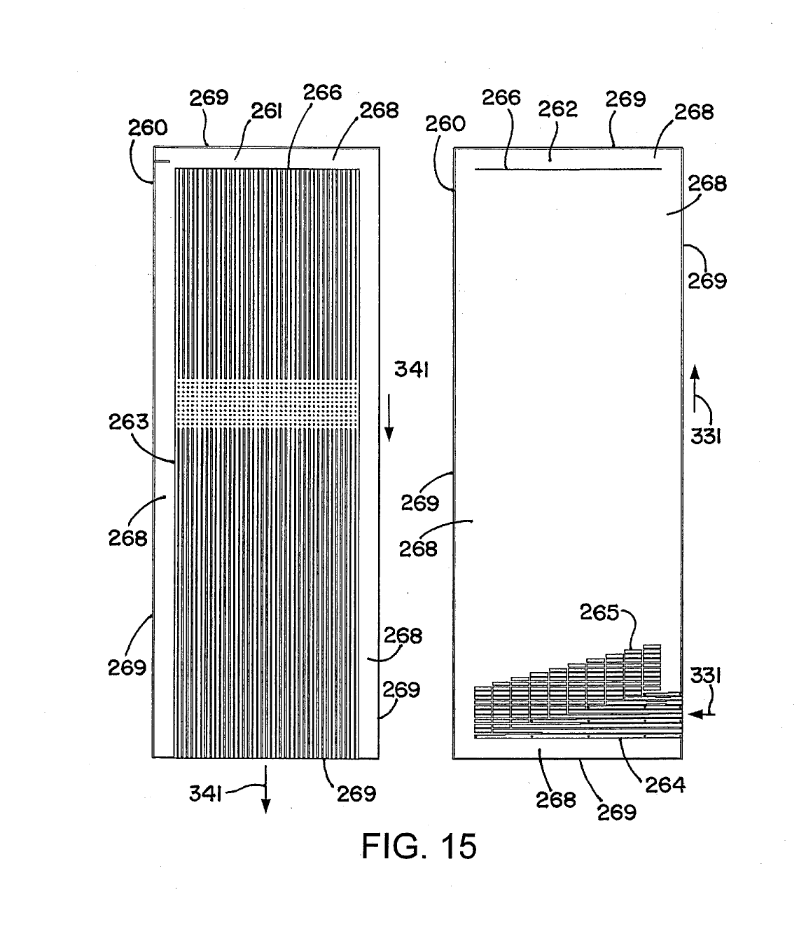

Plate 260 is illustrated in FIG. 15. The top surface 261 includes microchannels 263 which are used to provide for the flow of exhaust in the direction indicated by arrows 341. The bottom surface 262 includes internal manifold 263 which is used to provide for the flow of air in the direction indicated by arrow 331. The bottom surface 262 also includes surface features 265 to provide for redistribution of the flow of the air. The plate 260 includes opening or slot 266 to provide a u-turn for the flow of exhaust from the microchannels 283 of plate 280 to microchannels 254 of plate 250. Each side of the plate 260, that is plate surfaces 261 and 262, has a border 268. The plate 260 includes a peripheral edge 269 on each of the four sides of the plate. In the formation of the stack 100, or of the repeat unit 110, each of the peripheral edges 269 has a welding material applied to it. When the welding material is applied, it will typically penetrate beyond the peripheral edge 269 in contact with a portion of the border 268 on each side of the plate 260. During refurbishing, the welding material may be removed, for example, by milling, is grinding and/or cutting, from the peripheral edges 269 and as a result part of the border 268 may also be removed.

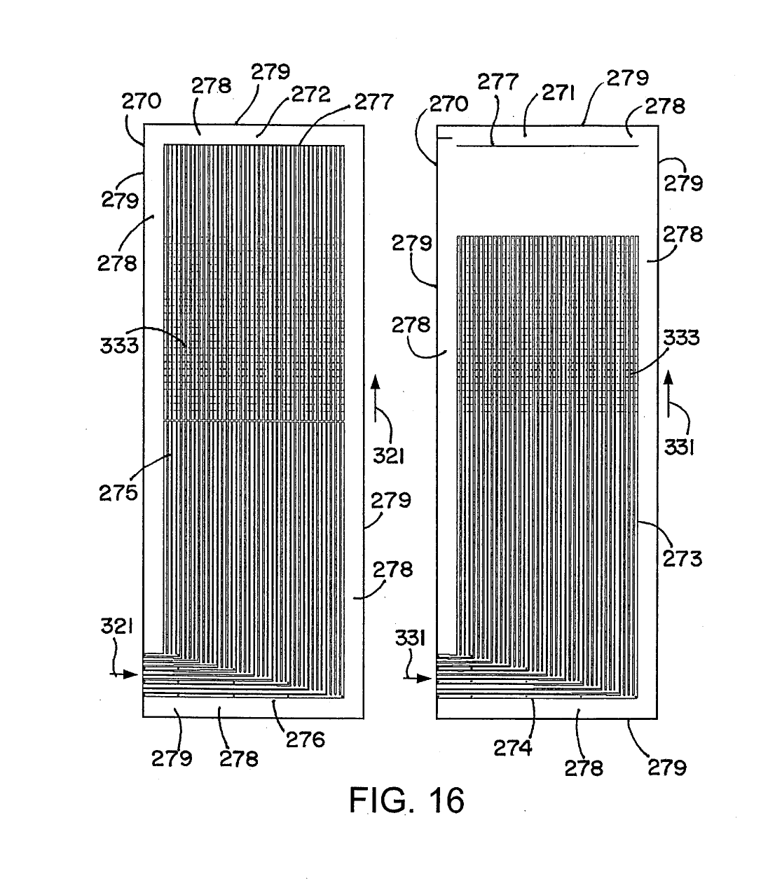

Plate 270 is illustrated in FIG. 16. The top surface 271 includes microchannels 273 and internal manifold 274 which are used to provide for the flow of air in the direction indicated by arrows 331. The bottom surface 272 includes microchannels 275 and internal manifold 276 which are used to provide for the flow of fuel in the direction indicated by arrows 221. The plate includes openings or jets 333 to provide for the flow of air from the microchannels 273 through the plate 270 into the microchannels 275 where the air may combine with the fuel to form a fuel-air mixture. The plate 270 includes opening or slot 277 to provide a u-turn for the flow of exhaust from the microchannels 275. Each side of the plate 270, that is plate surfaces 271 and 272, has a border 278. The plate 270 includes a peripheral edge 279 on each of the four sides of the plate. In the formation of the stack 100, or of the repeat unit 110, each of the peripheral edges 279 has a welding material applied to it. When the welding material is applied, it will typically penetrate beyond the peripheral edge 279 in contact with a portion of the border 278 on each side of the plate 270. During refurbishing, the welding material may be removed, for example, by milling, grinding and/or cutting, from the peripheral edges 279 and as a result part of the border 278 may also be removed.

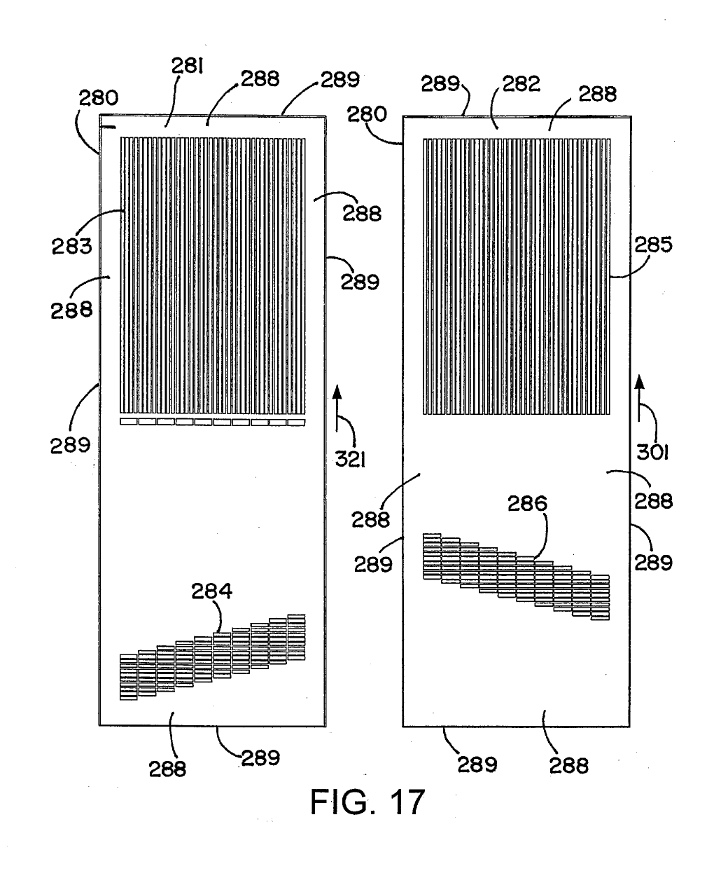

Plate 280 is illustrated in FIG. 17. The top surface 281 includes process microchannels 283, which are coated with the combustion catalyst, and surface features 284 for redistributing flow of the fuel. The bottom surface 282 includes microchannels 285, which are coated with an SMR catalyst, and surface features 286 for redistributing flow of the SMR reactants. Each side of the plate 280, that is plate surfaces 281 and 282, has a border 288. The plate 280 includes a peripheral edge 289 on each of the four sides of the plate. In the formation of the stack 100, or of the repeat unit 110, each of the peripheral edges 289 has a welding material applied to it. When the welding material is applied, it will typically penetrate beyond the peripheral edge 289 in contact with a portion of the border 288 on each side of the plate 280. During refurbishing, the welding material may be removed, for example, by milling, grinding and/or cutting from the peripheral edges 289 and as a result part of the border 288 may also be removed.

Plate 290 is illustrated in FIG. 18. The top surface 291 includes microchannels 293 and internal manifold 293A which may be used to provide for the flow of the SMR reactants in the direction indicated by arrows 301. The bottom surface 292 includes microchannels 294 and internal manifold 295 which may be used to provide for the flow of the SMR product in the direction indicated by arrows 311. The microchannels 294 include surface features 296 which may be used to disrupt the flow of product flowing through the process microchannels 294. The microchannels 293 include reaction zone 297 wherein a catalyst for the SMR reaction is coated on the microchannels. The reactants, which may comprise a mixture of methane and steam, flow through the reaction zone 297, contact the catalyst and react to form product. The product may comprise a mixture of carbon monoxide and hydrogen. The plate 290 includes u-turn opening 297A to provide for the flow of product from the process microchannels 297 to the process microchannels 294. Each side of the plate 290, that is plate surfaces 291 and 292, has a border 298. The plate 290 includes a peripheral edge 299 on each of the four sides of the plate. In the formation of the stack 100, or of the repeat unit 110, each of the peripheral edges 299 has a welding material applied to it. When the welding material is applied, it will typically penetrate beyond the peripheral edge 299 in contact with a portion of the border 298 on each side of the plate 290. During refurbishing, the welding material may be removed, for example, by milling, grinding and/or cutting, from the peripheral edges 299 and as a result part of the border 298 may also be removed.

The SMR catalyst layers 350, 351, 352 and/or 353, and/or the combustion catalyst layers 360 and/or 361 may be directly washcoated on the interior walls of the microchannels, or grown on the walls from solution. The catalyst layers may be selectively sprayed on the walls of the microchannels with the use of a mask to keep the coating in only desired locations, e.g., within the flow channels and substantially out of the interfacial area between plates that are not a target flow path. An advantage of the invention is that the catalyst layers may be applied to the plates before the plates are stacked. The cross-sectional area of each catalyst may occupy from about 1 to about 99%, or from about 10 to about 95% of the cross-sectional area of the microchannels. The catalyst layers may have a surface area, as measured by BET, greater than about 0.5 m²/g, or greater than about 2 m²/g. The catalyst may have any surface area and is particularly advantageous in is the range of about 10 m²/g to 1000 m²/g, or from about 20 m²/g to about 200 m²/g.

The catalyst layers may comprise an interfacial layer and a catalyst material deposited on or mixed with the interfacial layer. A buffer layer may be positioned between the microchannel surface and the interfacial layer. The buffer layer may be grown or deposited on the microchannel surface. The buffer layer may have a different composition and/or density than the interfacial layer. The buffer layer may comprise a metal oxide or metal carbide. The buffer layer may comprise Al₂O₃, TiO₂, SiO₂, ZrO₂, or combination thereof. The Al₂O₃ may be α-Al₂O₃, γ-Al₂O₃ or a combination thereof. The buffer layer may be used to increase the adhesion of the interfacial layer to the microchannel. The interfacial layer may comprise nitrides, carbides, sulfides, halides, metal oxides, carbon, or a combination thereof. The interfacial layer may provide high surface area and/or a catalyst-support interaction for supported catalysts. The interfacial layer may comprise any material that may be used as a catalyst support. The interfacial layer may comprise a metal oxide. Examples of metal oxides that may be used may include Al₂O₃, SiO₂, ZrO₂, TiO₂, tungsten oxide, magnesium oxide, vanadium oxide, chromium oxide, manganese oxide, iron oxide, nickel oxide, cobalt oxide, copper oxide, zinc oxide, molybdenum oxide, tin oxide, calcium oxide, aluminum oxide, lanthanum series oxide(s), zeolite(s) and combinations thereof. The interfacial layer may serve as a catalytically active layer without any further catalytically active material deposited thereon. The interfacial layer may be used in combination with a catalytically active material or layer. The interfacial layer may also be formed of two or more compositionally different sublayers. The interfacial layer thickness may range from about 0.5 to about 100 μm, or from about 1 to about 50 μm. The catalyst material may be deposited on the interfacial layer. Alternatively, the catalyst material may be simultaneously deposited with the interfacial layer. The catalyst material may be intimately dispersed on and/or in the interfacial layer. That the catalyst material may be “dispersed on” or “deposited on” the interfacial layer includes the conventional understanding that microscopic catalyst particles may be dispersed: on the interfacial layer surface, in crevices of the interfacial layer, and/or in open pores in the interfacial layer.

Alternatively, the SMR catalyst layers 350, 351, 352 and/or 353, and/or the combustion catalyst layers 360 and/or 361 may each comprise a fixed bed of is particulate solids. The median particle diameter may be in the range from about 1 to about 1000 μm, or from about 10 to about 500 μm.

The SMR catalyst layers 350, 351, 352 and/or 353, and/or the combustion layers 360 and 361 may comprise a foam for retaining catalyst particles. The catalyst layers may comprise coated foams, including graphite foams, silicon carbide, metal (e.g., Fecralloy which is an alloy comprising Fe, Cr, Al and Y), ceramic, and/or internal coatings of grapheme for high thermal conductivity coatings.

The SMR and/or combustion catalysts may be supported on porous support structures such as foams, felts, wads or a combination thereof. The term “foam” is used herein to refer to a structure with continuous walls that include pores positioned along the length or the structure or throughout the structure. The pores may be on the surface of the continuous walls and used for adhering catalyst material (e.g., catalyst metal particles) to the walls of the foam structure. The term “felt” is used herein to refer to a structure of fibers with interstitial spaces there between. The term “wad” is used herein to refer to a structure of tangled strands, like steel wool.

The catalyst may be supported on a monolith, honeycomb structure, fin structure comprising one or more fins or a microgrooved support.

The SMR catalyst layers 350, 351, 352 and/or 353, and/or the combustion layers 360 and 361 may comprise graded catalysts. The graded catalysts may have varying turnover rates of catalytically active sites. The graded catalysts may have physical properties and/or a form that varies as a function of distance along the reaction path or location in the layer.

The stack 100 or repeat unit 110 may be assembled by stacking the plates one above another in the desired order. The stack may then be compressed to bring the plates into contact and reduce voids between plates. Compression may be applied with the use of a clamped fixture applying a load with a bolt assembly or through the use of an external press to apply a load to the stack. The plates may then be joined together by welding the peripheral edge of each plate to the peripheral edge of the next adjacent plate. This may be done on each of the four sides of the stack. In this manner a peripheral seal may be provided for the stack. The clamped feature or external press may be removed after the welding is completed. The thickness of each weld may be up to about 10 mm, or in the range is from about 0.25 to about 10 mm, or in the range from about 0.25 to about 8 mm, or in the range from about 0.25 to about 6.5 mm, or from about 0.25 to about 5 mm, or from about 0.5 to about 3 mm, or from about 0.75 to about 3 mm, or from about 1 to about 2 mm, or from about 1 to about 1.5, or about 1.27 mm. It is advantageous to use welds that are as thin as possible to allow for refurbishment as many times as possible. The welding material, which may be in the form of a welding wire, may comprise any metal or metal alloy. The welding material may comprise steel (e.g., stainless steel, carbon steel, and the like); aluminum; titanium, nickel; platinum; rhodium; copper; chromium; alloys containing any of the foregoing metals; monel; inconel; brass; or a combination of two or more thereof. The welding material and the plates may be made of the same metal or metal alloy; or a different metal or metal alloy. The plates and the welding material may comprise Inconel 617, which is discussed below. The welding technique may comprise tungsten inert gas welding, metal inert gas welding, electron beam welding, laser welding, and the like. Laser welding may be especially advantageous.

An advantage of this method of manufacturing is that the surface preparation requirements that would be required for diffusion bonding and/or brazing may be eliminated. Surfaces must be very clean and flat for a quality diffusion bond and/or braze. Elimination of the brazing and/or bonding step also eliminates the need to heat the assembled stack to a high temperature as required for diffusion bonding and/or brazing. The energy required to heat and cool the stack for brazing and/or bonding would be significant, as would be the time required to heat and cool the stack for bonding or brazing time without incurring undue strain and resulting deformation. With the inventive method of manufacture, the use of bonding and/or brazing steps may be eliminated, and thus the resulting microchannel processor may be manufactured with high quality for a lower cost and in less time.

The microchannel processor may be refurbished by removing the stack 100 from the pressurized containment vessel, and removing the welded manifolds from the stack. The stack 100 may then be refurbished by removing the welding material from the peripheral edges of the plates; separating the plates; correcting defects in the plates; reforming the stack of plates; and welding the peripheral edge of each plate to the peripheral edge of the next adjacent plate to provide a new perimeter seal for the stack. The welding material may be removed using any conventional is technique such as milling. When the stack 100 contains one or more catalysts, the catalysts may be replaced and/or regenerated prior to reforming the stack. Individual plates that cannot be repaired may be replaced.

It is desirable to use a relatively thin weld on the peripheral edges of each of the plates when assembling the stack so as to limit the penetration of the peripheral welds. By limiting the penetration of the peripheral welds, the plates 200, 201, 220, 230, 240, 250, 260, 270, 280 and 290 may undergo numerous refurbishment procedures before the border of each of the plates is reduced to the point where the plates are no longer functional. For example, the border for each plate may have a thickness of about 15 mm, and if 1.5 mm of the border is milled away during each refurbishment, the plates may be refurbished ten times before being discarded.

In an alternate embodiment, one or more of the plates 200, 210, 220, 230, 240, 250, 260, 270, 280 and/or 290 may include internal welding to prevent the flow of fluid from one microchannel to another microchannel in the same plate. The internal welding may be applied using a laser welding machine. The welding machine may be programmed, automated or semi-automated to follow the desired microchannel walls on each of the plates, with the plates being internally welded prior to applying the peripheral welding. A welding wire made of the same material as the plates may be used.

With the SMR reaction, methane and steam are reacted in the presence of a catalyst to form a mixture of carbon monoxide and hydrogen according to the following chemical equation:

CH₄+H₂O→CO+3H₂

The reactant mixture may also include one or more of hydrogen, nitrogen, carbon monoxide, carbon dioxide, and the like. The product formed by this reaction may be referred to as synthesis gas or syn gas. The SMR reaction is an endothermic reaction which requires heating. The heat for the reaction may be supplied by a combustion reaction conducted in the heat exchange layer. The combustion reaction may involve the reaction of a fuel with oxygen or an oxygen source. The fuel may comprise hydrogen, methane, a hydrocarbon fuel (e.g., diesel fuel, fuel oil, biodiesel, and the like), or a mixture of two or more thereof. The oxygen source may comprise oxygen, air, oxygen enriched air, or a gaseous mixture comprising oxygen and an inert gas (e.g., helium, argon, etc.).

The SMR catalyst may comprise any SMR catalyst. The active catalyst material or element for the SMR catalyst may comprise Ni, Ru, Rh, Pd, Ir, Pt, or a mixture of two or more thereof. The active catalyst material or metal may be supported by Al₂O₃, MgO, MgAl₂O₄, CeO₂, SiO₂, ZrO₂, TiO₂, or a combination of two or more thereof.

The combustion catalyst may comprise any combustion catalyst. The active catalyst material or element may comprise one or more noble metals such as Pt, Rh, Pd, Co, Cu, Mn, Fe, Ni; oxides of any of these metals, perovskites and/or aluminates. The combustion catalyst may be accompanied by an activity-enhancing promoter such as Ce, Tb or Pr, their oxides, or a combination of two or more thereof.

The combustion active catalyst material or element may be supported by any suitable support. The support may comprise Al₂O₃, MgO, MgAl₂O₄, SiO₂, ZrO₂, TiO₂, or a combination of two or more thereof.

When a catalyst is employed in the microchannels, the microchannels may be characterized by having a bulk flow path. The term “bulk flow path” refers to an open path (contiguous bulk flow region) within the process microchannels. A contiguous bulk flow region allows rapid fluid flow through the microchannels without large pressure drops. In one embodiment, the flow of fluid in the bulk flow region may be laminar. In an alternate embodiment, the flow of fluid in the bulk flow region may be in transition or turbulent. In yet another embodiment, the flow may have two or more flow regimes throughout the flow circuit, whereby the flow in at least a portion of the flow path is in a transition flow regime as defined by a Reynolds number between about 2000 and about 5000. The bulk flow regions may comprise from about 5% to about 95%, and in one embodiment about 30% to about 80% of the cross-section of the microchannels that contain a catalyst.

Heating or cooling may be provided in the heat exchange layer using methods other than a combustion reaction. When heating or cooling other than by the use of a combustion reaction is employed, a heat exchange fluid, which may be any fluid, may be used. The fluid may comprise air, steam, liquid water, steam, gaseous nitrogen, other gases including inert gases, carbon monoxide, molten salt, oils such as mineral oil, a gaseous hydrocarbon, a liquid hydrocarbon, heat exchange fluids such as Dowtherm A and Therminol which are available from Dow-Union Carbide, or a mixture of two or more thereof. “Dowtherm” and “Therminol” are trademarks. The heat exchange fluid may comprise a stream of one or more of the reactants and/or the product.

The heat exchange channels may comprise process channels wherein an endothermic or an exothermic process is conducted. These heat exchange channels may be microchannels. The process conducted in the heat exchange channels may comprise a chemical reaction of the opposite thermicity to the reaction conducted in the process microchannels. For example, a SMR reaction, which is an endothermic reaction may be conducted in the process microchannels, and a combustion reaction, which is an exothermic reaction, may be conducted in the heat exchange channels. Examples of endothermic processes that may be conducted in the heat exchange channels may include dehydrogenation or reforming reactions. The exothermic reactions may include combustion reactions, other exothermic oxidation reactions, and the like. The use of an exothermic or endothermic reaction in the heat exchange channels for heating or cooling may provide an enhanced heating or cooling effect that may enable a typical heat flux of roughly on an order of magnitude or more above that which would be provided without the exothermic or endothermic reaction.

The heat exchange fluid may undergo a partial or full phase change as it flows through the heat exchange channels. This phase change may provide additional heat removal from the process microchannels beyond that provided by convective cooling. For a liquid heat exchange fluid being vaporized, the additional heat being transferred from the process microchannels may result from the latent heat of vaporization required by the heat exchange fluid. An example of such a phase change would be a heat exchange fluid such as oil or water that undergoes partial boiling.

The heat exchange fluid in the heat exchange channels may have a temperature in the range from about 100° C. to about 800° C., or from about 250° C. to about 500° C. The difference in temperature between the heat exchange fluid and the process fluids in the process microchannels may be up to about 50° C., or up to about 30° C., or up to about 10° C. The residence time of the heat exchange fluid in the heat exchange channels may range from about 1 to about 1000 ms, or about 1 to about 500 ms, or from 1 to about 100 ms. The pressure drop for the heat exchange fluid as it flows in the heat exchange channels may be up to about 0.01 MPa/cm, or up to about 10 MPa/cm. The flow of the heat exchange fluid in the heat exchange channels may be laminar or in transition. The Reynolds Number for the flow of heat exchange fluid in the heat exchange channels may be up to about 50,000, or up to about 10,000, or up to about 2300, or in the range of about 10 to about 2000, or about 10 to about 1500.

The reactants may flow in the reaction zones in contact with the catalysts to produce a Reynolds number up to about 100000, or up to about 10000, or up to about 100. The Reynolds number may be in the range from about 200 to about 8000.

The heat flux for heat exchange in the microchannel processor may range from about 0.01 to about 500 watts per square centimeter of surface area of the heat transfer walls (W/cm²) in the microchannel processor, or from about 0.1 to about 350 W/cm², or from about 1 to about 250 W/cm², or from about 1 to about 100 W/cm², or from about 1 to about 50 W/cm².

The contact time of the reactants with the catalyst (including SMR and combustion catalysts) in the microchannels may range from about 1 to about 2000 milliseconds (ms), or from 1 to about 1000 ms, or from about 1 to about 500 ms, or from about 1 to about 250 ms, or from about 1 to about 100 ms, or from about 1 to about 50 ms, or from about 2 to about 1000 ms, or from about 2 to about 500 ms, or from about 2 to about 250 ms, or from about 2 to about 100 ms, or from about 2 to about 50 ms.

The gas hourly space velocity (GHSV) for the flow of fluids in the microchannels may be in the range from about 500 to about 2,000,000 hr⁻¹.

The pressure drop for the fluids as they flow in the microchannels may range up to about 0.01 MPa per centimeter of length of the microchannel (MPa/cm), or up to about 0.1 MPa/cm, or up to about 1 MPa/cm, or up to about 10 MPa/cm.

The flow of the process fluids in the microchannels may be laminar or in transition, or turbulent. The Reynolds Number for the flow of fluids in the microchannels may be up to about 10,000, or up to about 5000, or up to about 2500, or up to about 2300, or in the range of about 100 to about 5000, or in the range from about 100 to about 3500, or in the range from about 100 to about 2300.

The superficial velocity for fluid flowing in the microchannels of the process layer may be at least about 10 meters per second (m/s), or in the range from about 10 to about 200 m/s, or in the range from about 20 to about 150 m/s, or in the range from about 30 to about 100 m/s, or in the range from about 50 to about 90 m/s.

The welded SMR reactor of the invention provides for advantages relating to enhanced or increased levels of heat transfer. The total reaction heat per unit contact time in the catalyst section of the reactor may be in the range from about 90 to about 150 kW/ms, or from about 110 to about 130 kW/ms. The total reaction heat per unit contact time in the reactor section of the reactor may be in the range from about 55 to about 75 kW/ms, or from about 60 to about 70 kW/ms. The total reaction heat per unit contact time in the overall reactor core of the reactor may be in the range from about 30 to about 50 kW/ms, or from about 30 to about 40 kW/ms. The total reaction heat per unit pressure drop for the reactor may be in the range from about 2 to about 20 W/Pa, or from about 2 to about 10 W/Pa, or from about 2 to about 5 W/Pa.

### Example 1

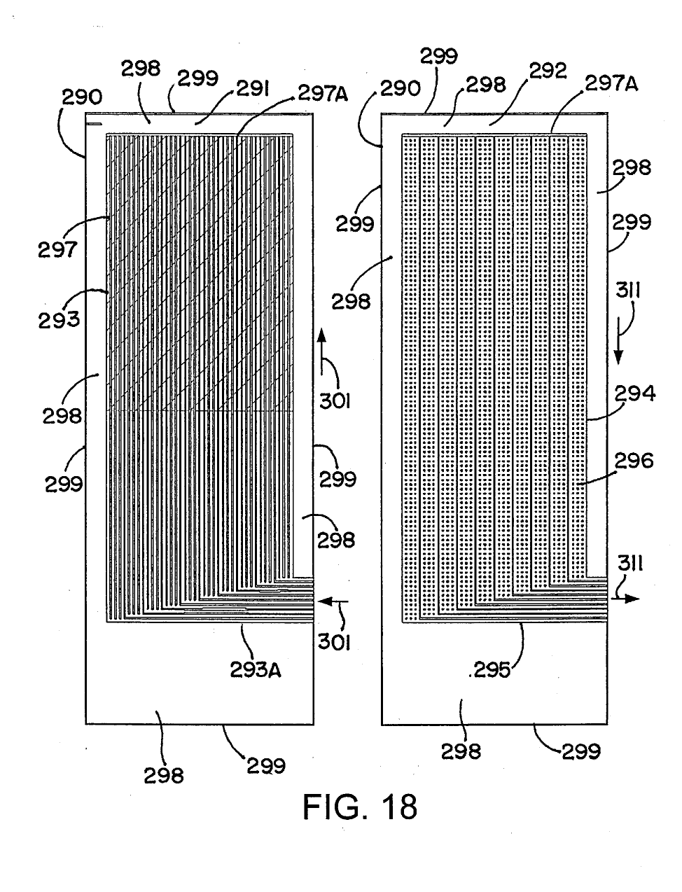

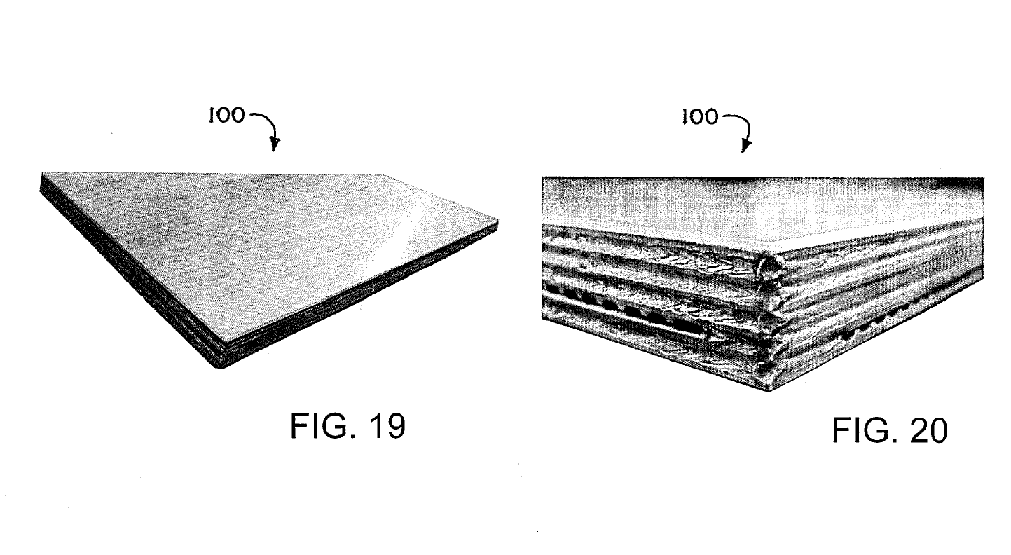

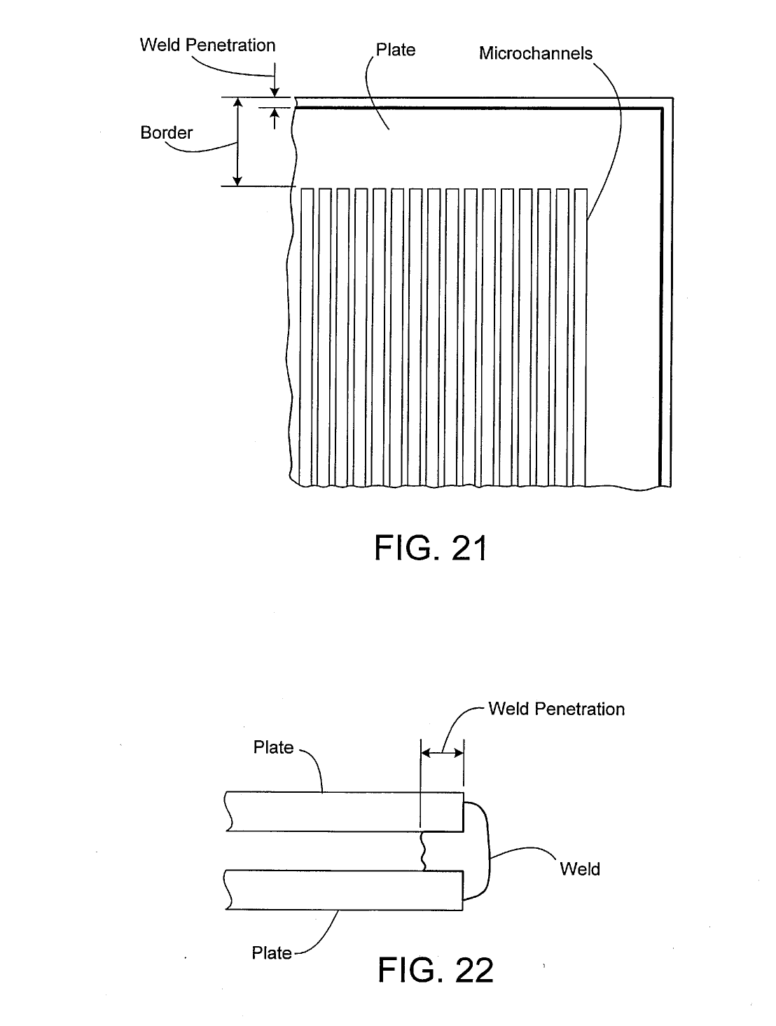

An SMR process using a microchannel reactor of the type illustrated in FIGS. 1-20 is simulated using Chemcad. Chemcad is a process simulation software program available from Chemstations Deutschland GmbH. The reactor employs 8 of the repeat units 110 shown in FIGS. 7 and 8. Each repeat unit has 10 plates and thus a total of 80 plates are provided by the repeat unit. An 81^{st} plate is joined to surface 292 of plate 290 at the bottom of the stack. Each of the 81 plates has a length of 29 inches (73.66 cm), a width of 10.74 inches (27.28 cm) and a thickness of 0.125 inch (3.175 mm). The surface area of each plate is 2009.4 cm². The total stack height is 10.125 inches (25.72 cm). The peripheral edges of the plates are welded together using laser welding. Each peripheral edge of each plate is welded to the peripheral edge of the next adjacent plate. The average weld penetration is 1.27 mm. The ratio of the average surface area of each plate (2009.4 cm²) to the average penetration of the welds is (1.27 mm) is 1580 cm²/mm.

Each of the plates as well as the weld material is made of Inconel 617 which is a metal alloy containing nickel, chromium, cobalt, molybdenum and aluminum. Inconel 617 is available from A-1 Wire Tech, Inc. and has the following composition and properties:

Chemical Composition, weight ° A):

### Ni.—44.5 min.

### Cr—20.0-24.0

### Co—10.0-15.0

### Mo—8.0-10.0

### Al—0.8-1.5

### C—0.05-0.15

### Fe—3.0 max.

### Mn—1.0 max.

### Si—1.0 max.

### S—0.015 max.

### Ti—0.6 max.

### Cu—0.5 max.

### B—0.006 max.

### Rupture Strength (1000 h)

| | MPa |
|---|---|
| 650° C. | 320 |
| 760° C. | 150 |
| 870° C. | 58 |
| 980° C. | 25 |
| 1095° C. | 10 |

### Physical Constants and Thermal Properties:

Density: 8.36 mg/m³

### Melting Range: 1330-1380° C.

### Specific Heat: 419 J/kg·° C.

### Thermal Conductivity: 13.6 W/m·° C.

The microchannels in each of the plates have a depth of 0.040 inch (1.016 mm). The width of each microchannel is 0.160 inch (4.064 mm). Each of the openings or jets in the heat exchange walls between the air channels and fuel channels has a diameter of 0.015 inch (0.381 mm).

The SMR reactor capacity is about 3500 SLPM of methane or natural gas feed when 640 process microchannels are used for the SMR reaction. The SMR reactor may be used to produce synthesis gas for use in one, two or more Fischer-Tropsch reactors operated in series with intermediate process collection. The Fischer-Tropsch reactors may be used to produce synthetic fuel. The synthesis gas may be advanced through an intermediate process unit (e.g., a membrane or other unit operation) prior to the Fischer Tropsch reactors to reduce the hydrogen to carbon monoxide ratio to about 2:1. The steam to carbon ratio for the SMR reactor is about 2.3:1 at the reactor inlet. The steam to methane ratio is 2:1. For the combustion reaction, about 15% excess air is used. A range of about 5% to about 50% excess air may be used. Higher levels of excess air may be used, but the use of such higher levels may be less efficient due to the need to preheat the unused air. Process equilibrium for the conversion of methane in the SMR reaction is 76.1% at a pressure of 223.2 psig (1.54 MPa) and a temperature of 850° C. CO/(CO+CO₂) at 223.2 psi (1.54 MPa) and 850° C. is 68.8%. The reactor core pressure drop is up to 60 psi (0.414 MPa) on the SMR process side, and up to 34 psid (0.234 MPa) on the fuel/air side. The nominal design basis for the reactor is shown in the following Table 1.

TABLE 1

| | | | | | |
|---|---|---|---|---|---|
| Component | Reactant | Product | Fuel | Air | Exhaust |

Stream Composition - Mole Fraction

| | | | | | |
|---|---|---|---|---|---|
| Hydrogen | 3.77% | 48.44% | 83.95% | | 0.00% |
| Oxygen | 0.00% | 0.00% | 0.00% | 21.00% | 2.22% |
| Nitrogen | 2.58% | 1.80% | 0.50% | 79.00% | 65.11% |
| Water | 56.69% | 23.94% | 1.38% | | 28.17% |
| Carbon Monoxide | 0.05% | 14.50% | 3.22% | | 0.00 |
| Carbon Dioxide | 8.56% | 6.58% | 8.89% | | 4.50% |
| Methane | 28.35% | 4.74% | 2.05% | | 0.00% |
| Total | 100.00% | 100.00% | 100.00% | 100.00% | 100.00% |

Stream Flow Rates

| | | | | | |
|---|---|---|---|---|---|
| Reactor (kg/hr) | 649.74 | 649.73 | 68.77 | 713.78 | 782.57 |
| Layer (kg/hr) | 40.609 | 40.608 | 4.298 | 44.611 | 48.910 |
| Channel (kg/hr) | 1.0152 | 1.0152 | 0.1075 | 1.1153 | 1.2228 |

Stream Temperature (° C.)

| | | | | | |
|---|---|---|---|---|---|
| Inlet/Outlet Reactor | 281 | 466 | 156 | 191 | 461 |
| HX-M2M Interface | 348 | 506 | 397 | 317 | N/A |
| RX-HX Interface | 544 | 711 | 606 | 541 | 790 |

Stream Pressure, psig, (MPa)

| | | | | | |
|---|---|---|---|---|---|
| Inlet/Outlet Reactor | 231.3 (1.59) | 214.2 (1.48) | 22.3 (0.15) | 31.7 (0.22) | 0.8 (0.0055) |
| HX-FD Interface* | 224.8 (1.55) | 221.2 (1.53) | 21.0 (0.15) | 23.0 (0.16) | 1.7 (0.012) |
| RX-HX Interface** | 224.4 (1.55) | 221.9 (1.53) | 20.7 (0.14) | 20.7 (0.14) | 3.0 (0.021) |
| U-turn | 223.2 (1.54) | 223.2 (1.54) | 6.2 (0.043) | 6.2 (0.043) | 6.2 (0.043) |

*Heat exchange/flow distribution interface.
**Reaction/heat exchange interface.

### Example 2

A four-channel full-length SMR welded reactor is built, operated, refurbished and subsequently operated. The reactor at full scale is forecasted to have a 20-year life with roughly 10 refurbishment cycles. The reactor mimics the internal features and length of a full-scale microchannel SMR. The refurbishment process includes manifold removal, plate separation, modifying and cleaning a select number of the plates, adding catalyst to the refurbished plates, and re-assembly. Reactor capacity and reaction performance are repeatable after refurbishment.

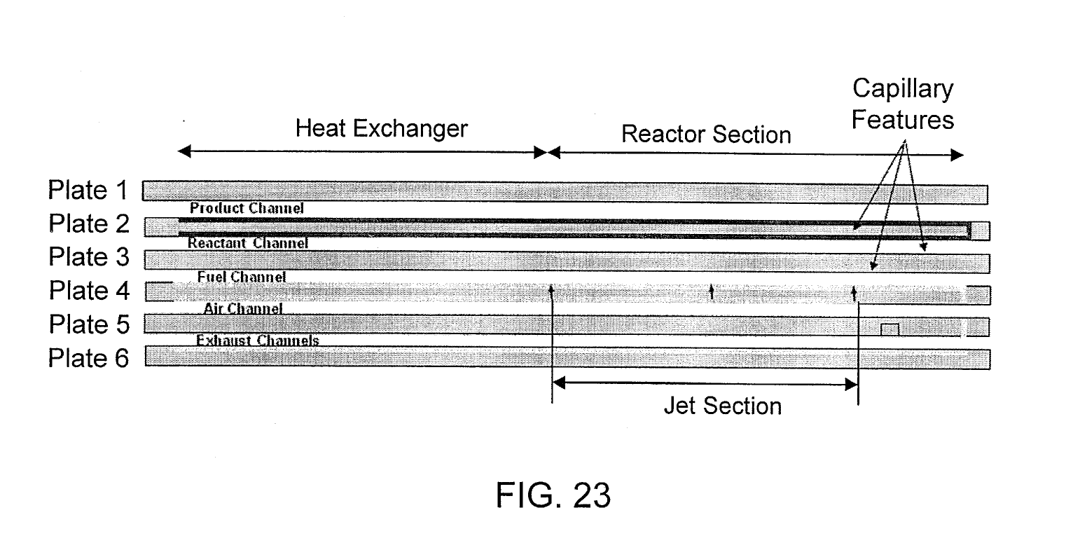

An overview of the reactor is shown in FIG. 23. Referring to FIG. 23, the reactor has two layers, namely, a process layer and combustion layer. The process layer includes reactant and product channels. The combustion layer includes fuel, is air and exhaust channels. An SMR reaction is conducted in the reactant and product channels. A combustion reaction is conducted in the fuel channel to provide heat needed for the SMR reaction.

The reactor is divided into three sections:
1. Heat exchanger—this section recuperates heat from the exhaust and product streams, and uses the heat to pre-heat the fuel, air and reactant streams.
2. Reactor section—in this section the SMR and combustion reactors are conducted.
3. Inlet section (not shown in FIG. 23)—this section provides for inlet/outlet connections and distribution of flow to the microchannels. The length of the heat exchanger section is 8 inches (20.3 cm). The length of the reactor section is 13 inches (33 cm). The reactor has four channels of each type (reactant, product, fuel, air and exhaust). The width of each channel is 0.16 inches (4.06 mm). The gap or height of each channel is 0.04 inch (1.02 mm).

Air flows from the air channels through circular openings or jets into the fuel channel. The air mixes with the fuel in the fuel channel to form a fuel-air mixture, which undergoes combustion to generate heat for the SMR reaction. The mixing of the air with the fuel is conducted in the jet section, the length of the jet section being 8.5 inches (21.6 cm). In the jet reaction, there are 26 axial locations spaced 0.34 inches (0.86 cm) apart from one another where at each location one or more jets are positioned. Each jet has a diameter of 0.015 inch (0.381 mm). At certain axial locations, there are multiple jets for the air distribution.

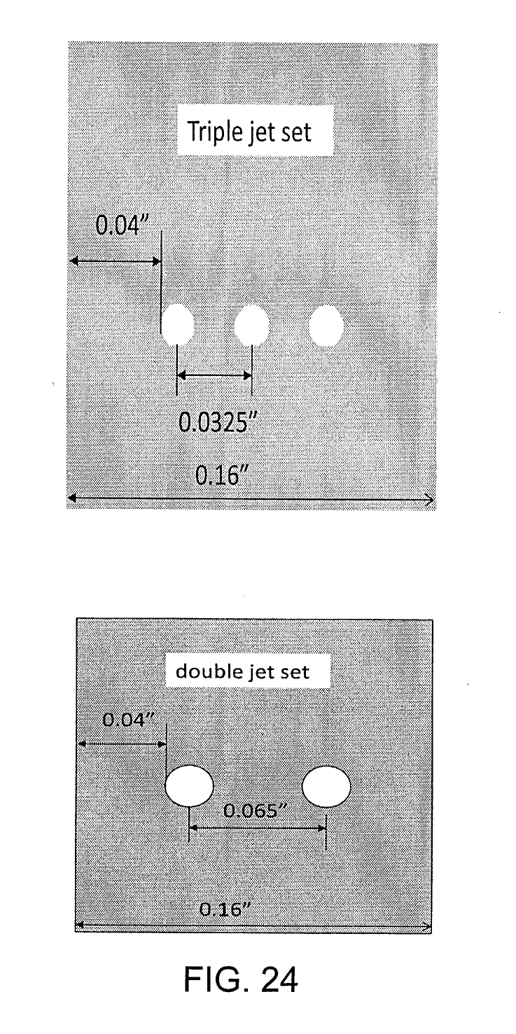

The schematic illustration in FIG. 24 shows the arrangement of two and three jets at an axial location across the 0.16 inch (4.06 mm) width of the fuel channel. For axial locations with one jet, the jet is located in the center of the width of the fuel is channel.

Exhaust from the combustion reaction flows through a U-turn bend as shown in FIG. 23, and enters the exhaust channel as an exhaust stream. The exhaust stream is used to pre-heat fuel and air streams in the heat exchanger section before leaving the reactor.

The heat generated by the combustion reaction is transferred to the reactant and product channels through a solid wall to heat the SMR reaction. The SMR reactants flow in the reactant channel, undergo reaction, in the presence of a catalyst and heat of combustion from the combustion reaction, to form the desired product which is synthesis gas. The product stream flows through the U-turn shown in FIG. 23. The product stream pre-heats the reactant stream in the heat exchanger section before leaving the reactor.

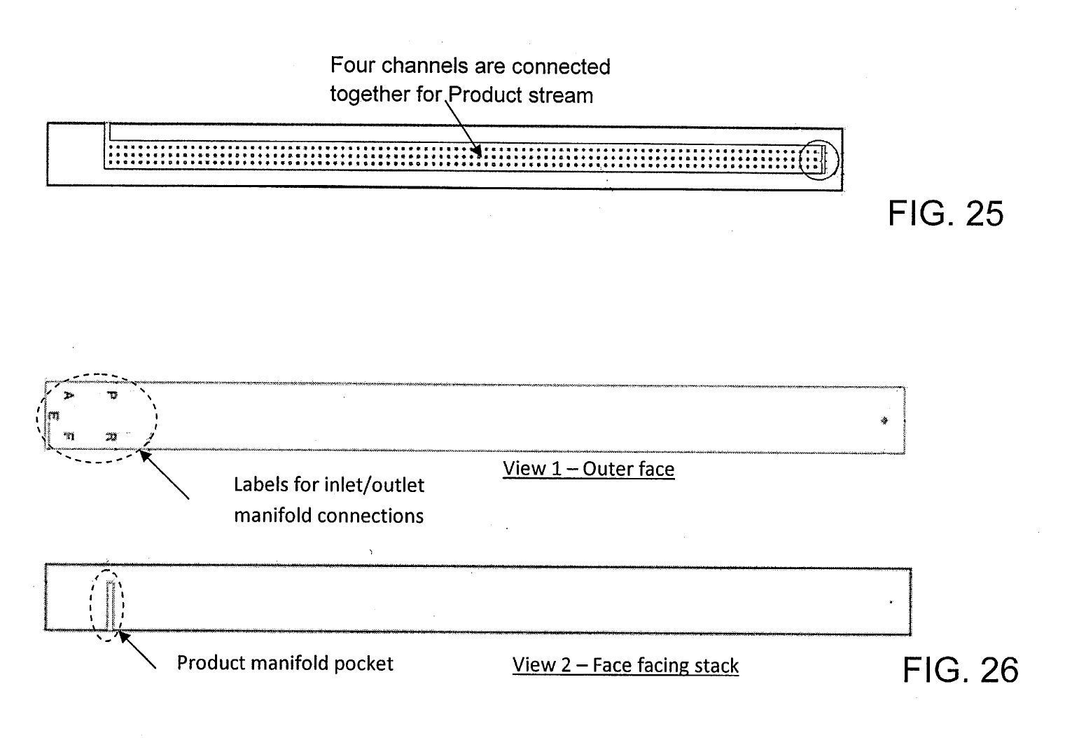

Connections among the four product channels are provided as shown in FIG. 25 with the use of open pillars to allow flow to redistribute if necessary in the event of channel blockage. Channel blockage may occur as a result from coking, catalyst delamination, or incoming particulates.

Capillary features are shown in FIG. 23. These features are in the form of shallow grooves. The grooves may have a depth in the range of about 10 to about 500 microns, or from about 30 to about 250 microns, or from about 50 to about 100 microns, or about 80 microns. The grooves may traverse part or all of the width of the indicated channels. These features are formed on the channel walls to provide better adherence for the catalyst.

FIG. 23 provides an overview of the reactor core. The reactor core shown in FIG. 23 is made using six plates stacked one above another. The microchannels are formed in the plates and the assembly of the plates forms the flow paths for the combustion and SMR streams. The plates are identified as follows:
Plate 1: Product or P plate
Plate 2: Reactant/Product or RP plate
Plate 3: Catalyst or Cat plate
Plate 4: Fuel/Air or FA plate
Plate 5: Air/Exhaust or AE plate
Plate 6: Exhaust or E plate

Plates 2 through 5 have a thickness of 0.125 inch (3.18 mm). Plates 1 and 6 have a thickness of 0.25 inch (6.35 mm).

### Plate 1: P-Plate

A schematic of the P-plate is shown in FIG. 26. The overall dimensions of the P-plate are 23.32″ (59.2 cm)×1.82″ (4.6 cm)×0.25″ (6.3 mm). This is the outermost plate in the SMR reactor core stack. On the outer face of the plate, labels R, P, A, F and E shows the locations for inlet/outlet manifolds for the reactant stream, product stream, air stream, fuel stream and exhaust stream respectively. On the face that faces the stack, a pocket of size 0.16″ (4.06 mm)×1.32″ (3.3 cm)×0.04″ (1.016 mm) is machined for a product manifold. The perimeter of the face facing the stack (shown in View 2, FIG. 26) is chamfered (0.031″ (0.8 mm)×45°) for weldment.

### Plate 2: RP-Plate

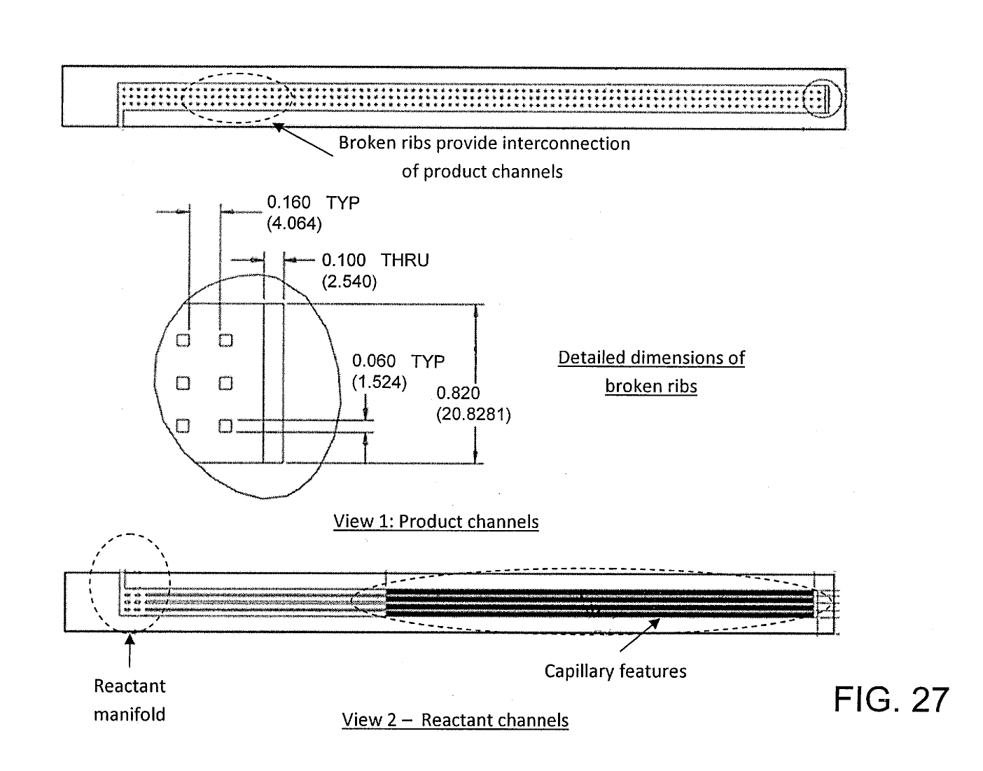

A schematic of the RP-plate is shown in FIG. 27. The overall dimensions of the RP-plate are 23.32″ (59.2 cm)×1.82″ (4.6 cm)×0.125″ (3.1 mm). This plate is located between the P-plate and the cat-plate. On the face (shown in View 1, FIG. 27) adjacent to P-plate, four product channels are machined. The wall between the product channels has connections for fluid communication. These are referred to as broken ribs. Dimensions for the broken ribs are shown in FIG. 27. The depth of the product channels is 0.04″ (1.016 mm). The total length of broken rib zone is 21.5″ (54.6 cm).

The other face of the RP plate, which faces the Cat-plate, has reactant channels as shown in View 2, FIG. 27. There are four reactant channels connected to reactant inlet manifold as shown in FIG. 27. The width and the depth of the four reactant channels and reactant manifold are 0.16″ (4.06 mm) and 0.04″ (1.016 mm), respectively. The four reactant channels are separated by 0.06″ (1.52 mm) wide ribs. In the reactor section of this plate, capillary features are machined. The length of the capillary feature section is 13″ (33 cm). This is shown in FIG. 27. SMR catalyst is applied to the capillary features and the side walls of the ribs separating the reactant channels. The perimeter of the plate is chamfered (0.031″ (0.79 mm)×45°) for weldment.

A through slot with dimensions 0.82″ (2.08 cm)×0.1″ (2.54 mm) is machined to allow combustion exhaust to flow to the exhaust channels.

### Plate 3: Cat-Plate

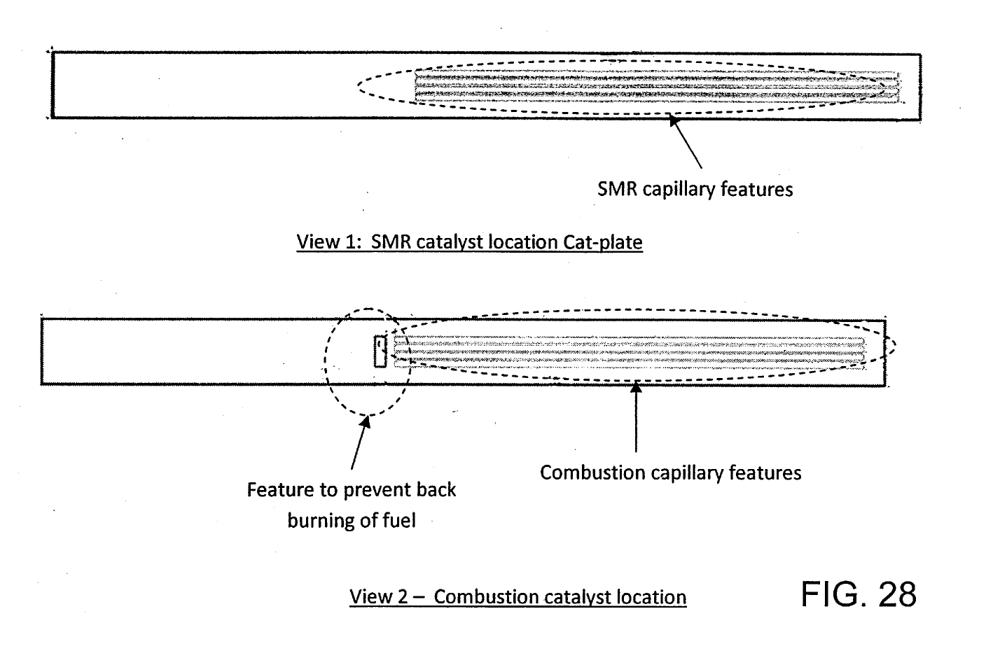

A schematic of the cat-plate is shown in FIG. 28. The overall dimensions of the cat plate are 23.32″ (59.2 cm)×1.82″ (4.6 cm)×0.125″ (3.1 mm). This plate is located between the RP plate and the FA plate. On the side facing RP plate, capillary features are machined as shown in View 1, FIG. 28. The zones where SMR catalyst is applied overlaps with the zones of capillary features on the RP plate in FIG. 27. The SMR catalyst is applied on the capillary features.

The side of the cat-plate that faces the FA plate also has capillary features. The capillary features in this zone replicate the capillary features on the other side (facing RP-plate) of the plate as shown in View 2, FIG. 28. A pocket of dimensions 0.82″ (2.08 cm)×0.3″ (7.6 cm)×0.02″ (0.51 mm) is machined 0.25″ (6.35 cm) away from the capillary features. After assembly of all the plates, this pocket prevents back burning of the fuel that could cause operational instability.

Holes are drilled in the thickness direction of the plate at 21 axial locations in the plate to measure temperatures during operation of the reactor. These holes are 0.034″ (0.86 mm) in diameter and 0.91″ (2.31 cm) deep.

The perimeter of the plate is chamfered (0.031″ (0.78 mm)×45°) for weldment.

### Plate 4: FA-Plate

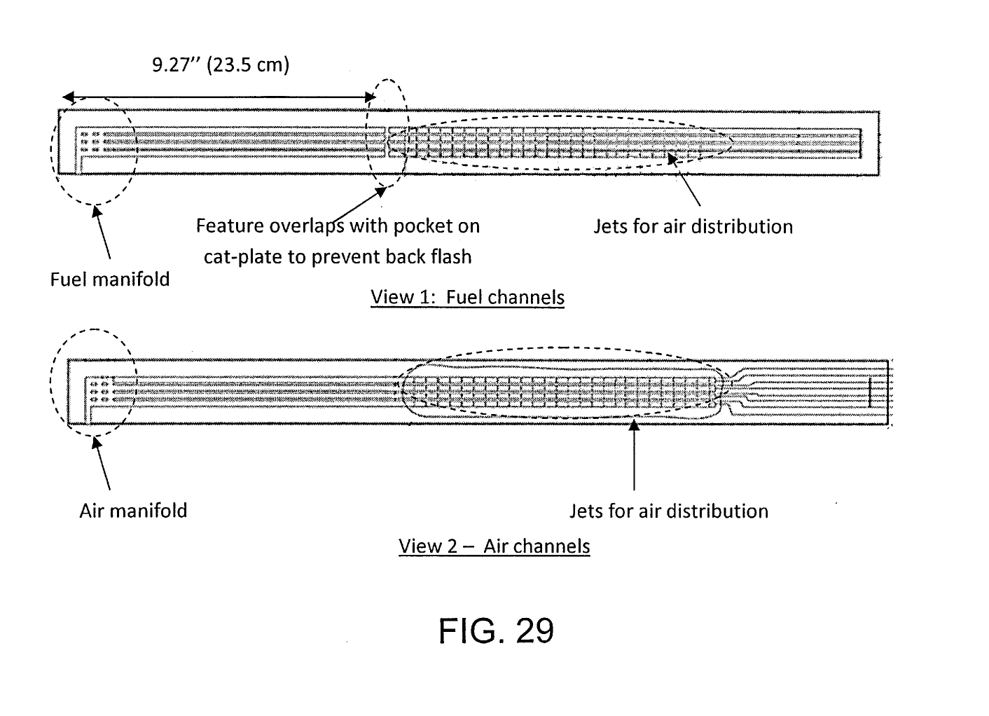

A schematic of the FA-plate is shown in FIG. 29. The overall dimensions of the FA-plate are 23.32″ (59.2 cm)×1.82″ (4.6 cm)×0.125″ (3.1 mm). This plate is located between the Cat-plate and the AE-plate.

On the side facing the Cat-plate, four fuel channels connected to a fuel manifold are machined. The width of the fuel manifold as well as fuel channels is 0.16″ (4.06 mm) and the depth of the manifold and channels is 0.04″ (1.016 mm). The length of the fuel manifold is 1.32″ (3.4 cm). The continuity of fuel channels is broken (as shown in FIG. 29) at 9.27″ (23.5 cm) from the shorter edge of the plate that is closest to the fuel manifold. The discontinuity in fuel channels overlaps with the pocket feature in the Cat-plate to prevent back burning of the fuel.

On the other side of the plate (facing the AE plate), four air channels connected to air manifold are machined. The dimensions (width and depth) of the manifold as well as channels are the same as the dimensions of the fuel channels and manifold.

The fuel and air channels are connected together by jets. The location of these jets is shown in FIG. 29. The diameter of each jet is 0.015″ (0.38 mm). There are 26 axial jet locations that are spaced 0.34″ (8.6 mm) apart. Some axial locations have multiple jets. A summary of number of jets at various axial locations and arrangement of jets is shown in Table 3.

A through slot with dimensions 0.82″ (2.1 cm)×0.04″ (1 mm) is machined to allow exhaust from the combustion reaction to flow to the exhaust channels.

The perimeter of plate is chamfered (0.031″ (0.8 mm)×45°) for weldment.

### Plate 5: AE-Plate

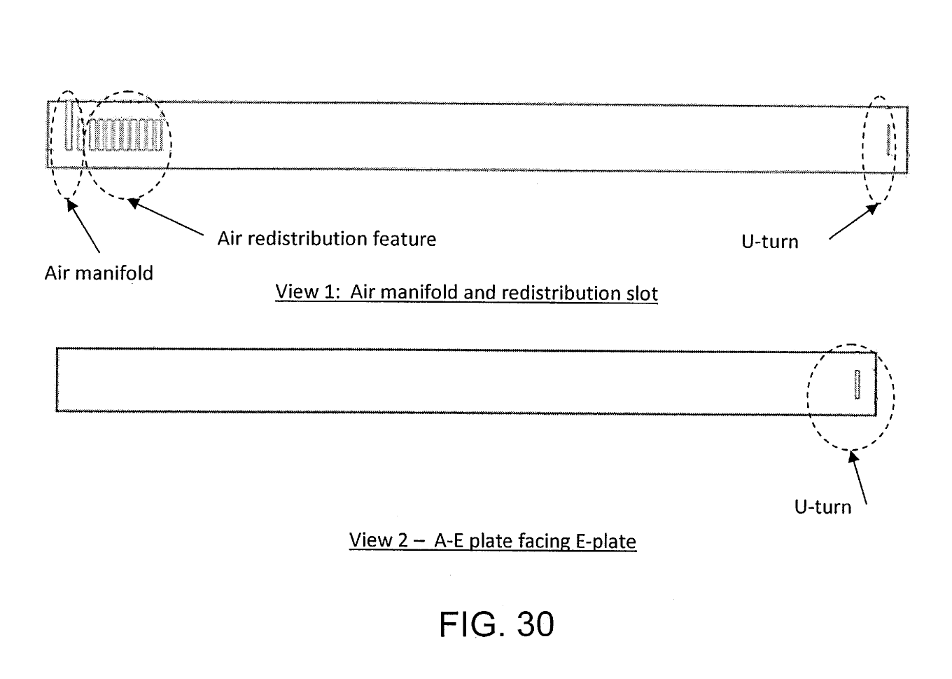

A schematic of the AE-plate is shown in FIG. 30. The overall dimensions of the AE plate are 23.32″ (59.2 cm)×1.82″ (4.6 cm)×0.125″ (3.1 mm). This plate is located between the FA-plate and the E-plate.

On the side of the AE plate facing FA plate, a manifold slot and 10 redistribution slots are machined as shown in FIG. 30 (View 1). The width of all the slots is 0.16″ (4.06 mm) and depth of the slots is 0.04″ (1.016 mm). The manifold slot on the AE plate overlaps with manifold slot on the FA-plate to form the manifold. The spacing between the air manifold slot and first redistribution slot is 0.16″ (4.06 mm) and the spacing between first redistribution slot and second redistribution slot is 0.16″ (4.06 mm). The spacing between other redistribution slots is 0.06″ (1.52 mm).

The other side of the AE plate (facing the E-plate), there are no features except for the through slot described below.

A through slot with dimensions 0.82″ (2.08 cm)×0.04″ (1.106 mm) is machined to allow combustion exhaust to flow to the exhaust channels.

The perimeter of plate is chamfered (0.031″ (0.8 mm)×45°) for weldment.

### Plate 6: E-Plate

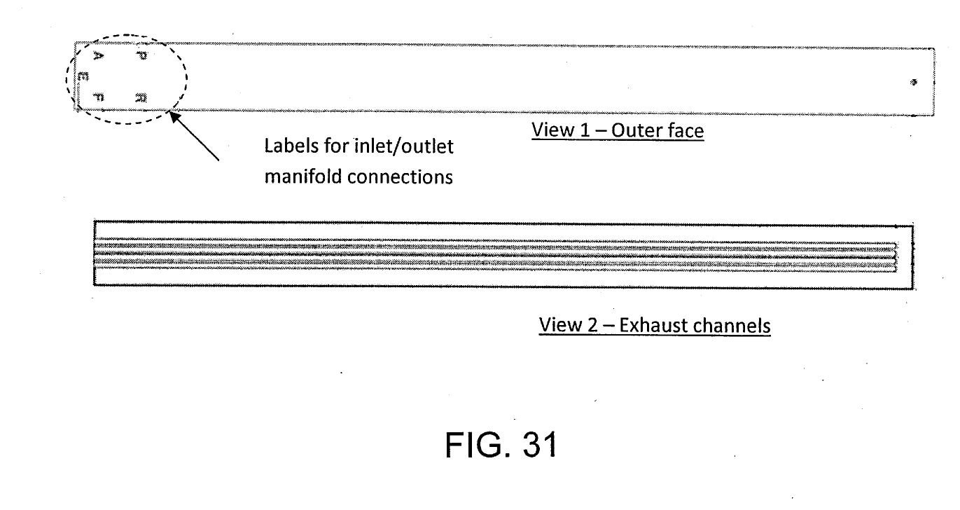

A schematic of the E-plate is shown in FIG. 31. The overall dimensions of the E-plate are 23.32″ (59.2 cm)×1.82″ (4.6 cm)×0.25″ (6.3 mm). This is the outermost plate in the SMR reactor core stack, farthest from the P-plate. On the outer face of the plate, labels R, P, A, F and E show the locations for inlet/outlet manifolds for reactant stream, product stream, air stream, fuel stream and exhaust stream, respectively. On the face that faces the stack, four exhaust channels are machined. Each channel is 0.16″ (4.06 mm) wide and 0.04″ (1.016 mm) deep. The length of the exhaust channels is 22.78″ (57.9 cm).

The perimeter of the face facing the stack (shown in View 2, FIG. 26) is chamfered (0.031″ (0.8 mm)×45°) for weldment.

Supports in the form of an exoskeleton are provided around the reactor core to support high process pressure for microchannel integrity. This is shown in FIG. 32 which is a schematic illustration of the final reactor.

The reactor is constructed using 0.125 inch (0.318 cm) thick Inconel 617 plates. The plates and the microchannel features in the plates are made by using conventional machining. Capillary features may be added using laser machining, photochemical milling or machining, or by other methods of metal removal. Jets may be fabricated using laser drilling.

After manufacturing of the plates and the features in the plates, the plates are aluminized using a chemical vapor deposition (CVD) aluminization process and heat treated at 1050° C. to form an adherent alumina scale. The alumina scale layer may prevent the plates from sticking during operation to facilitate or enable refurbishment.

After heat treatment, SMR catalyst (20% Rh on a support of 28% MgO-72% Al₂O₃ spinel) at about 30 mg/in² (4.65 mg/cm²) is coated on both sides of the process channels. Combustion catalyst (35 wt % Pt and 8 wt % Pd on fumed Al₂O₃ with lanthanum support) at an applied coating level of about 30 mg/in² (4.65 mg/cm²) is coated on the jet impingement or fuel wall using spray coating. The catalysts are applied to the open plates prior to welding. This method allows for direct access to the faces and the ability for quality control of the coated catalysts. Also, the direct access enables ease of refurbishment to strip spent catalyst and reapply. The open plates allow for the use of one or two or more catalysts within a process layer, or across a process plate, or from layer to layer to tailor or optimize process performance. The catalysts are calcined in situ at 400° C. prior to operation.

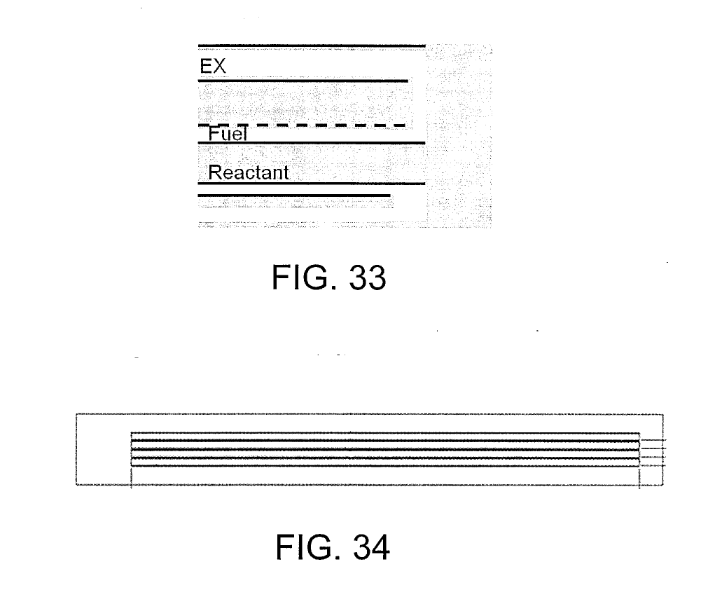

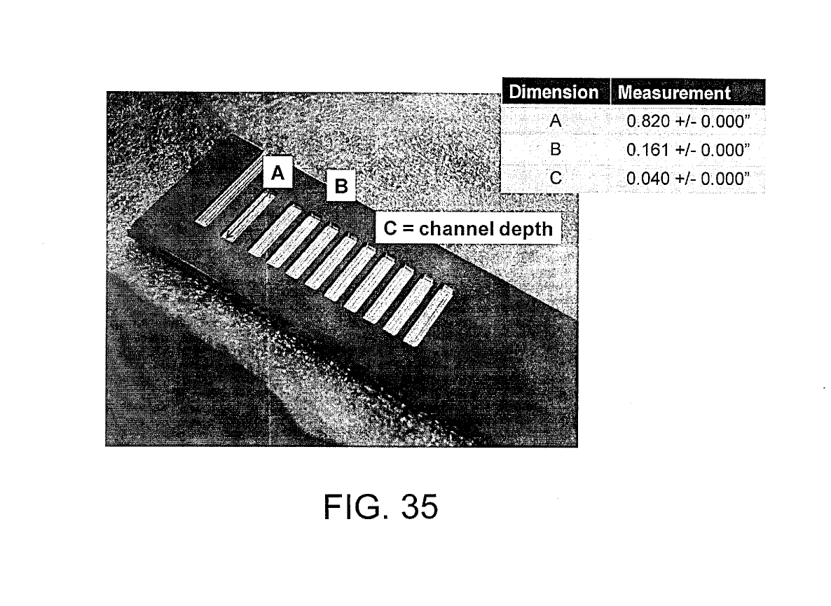

The catalysts (SMR and combustion) are applied in the reactor section only. A schematic showing the location of SMR and combustion catalyst is shown in FIG. 33. The SMR catalyst is spray coated on the capillary features as well as the side wall of the reactant channels formed by the RP plate and the Cat-plate. A mask made of carbon steel is used to facilitate catalyst coating. A schematic of the mask used for the SMR catalyst coating is shown in FIG. 34.

The combustion catalyst is coated on the capillary features in the fuel channel is formed by the cat-plate and the FA plate. The fuel wall of the FA plate is partially coated with catalyst. The exhaust channel formed by AE plate and E-plate are coated with combustion catalyst.

The plates are welded together to form the reactor core. Tungsten inert gas welding is used. Exterior welding is used where the peripheral edge of each plate is welded to the peripheral edge of the next adjacent plate. The welds have an average penetration from about 0.03 inch (0.762 mm) to 0.08 inch (2.032 mm). Each plate has a surface area of 272.3 cm. Thus, the ratio of the average surface area to the average weld penetration is from 134.0 to 357.4 cm²/mm. Prior to welding, aluminide is ground off at the edges. An exoskeleton in the form of support ribs, a macro-manifold and tubes are added to the core.

The reactor, which is in the form of a multichannel test device, consists of six CVD aluminized plates that are heat treated and catalyst coated prior to assembly. The refurbishment process includes removal of the exoskeleton, removal of exhaust manifolds and separation of the plates.

During refurbishment, the core is removed from the exoskeleton support. The next step is to remove the reactant, product, fuel, air and exhaust manifolds. The exhaust manifold is removed last. The first four manifolds require their 0.25 inch (0.635 cm) tubes to be removed first. A computer numerical control (CNC) milling machine that uses CAD input or programming logic in order to accurately machine parts, is used to machine the weld perimeter of each manifold. To do so, the weld perimeter of each manifold is machined down allowing the manifolds to be pulled away from the device. The exhaust manifold is also removed by machining away the weld. With the core free of manifolds, the plates are separated via milling of the perimeter welds. The initial milling targets removal of 40 mils (1.02 mm) of material. The plates appear to be separated in some areas, but could not be pulled apart. Another 20 mils (0.51 mm) of material is removed from the perimeter. The core is again clamped in place. All the plates are pulled apart with the use of pliers. A total of 60 mils (1.53 mm) of material is machined away to sufficiently remove the weld to allow the plates to be separated.

With the plates separated, each plate is inspected. The U-turn is modified during the refurbishment process—a rectangular insert is added in the U-turn to reduce the original size. This insert is welded in place without additional surface is preparation or treatment. The three combustion side plates (FA, AE and E) are modified.

All plates are cleaned using a low power and low frequency ultrasound in a deionized water bath, followed by an acetone bath. Each step is carried out for 30 minutes. No delamination or damage to the catalyst occurs. Catalyst is coated in part of the FA plate near the modified jets. A modified coating layout is applied over the first 16 jets, where catalyst is placed on the outer edges of the 0.16″ (4.064 mm) wide channel, 1 mm of catalyst coated on each side, and the center 2 mm left uncoated.

Combustion catalyst is coated on both the top and the bottom exhaust channel walls. The catalyst is coated across the full 0.16″ (4.064 mm) width of the each of the four channels. Catalyst is masked in the area that forms metal to metal contact between a wall and a rib that intervenes between channels.

The reactor is re-stacked after the above modifications are completed. There is some bowing in the P plate of about 0.2 inch (5.08 mm). This is mitigated by clamping the plates in place once aligned after stacking. The core is peripherally welded and a new exoskeleton support is welded to the stack.

The reactor is operated at high capacity and heat flux conditions. Two sets of operating conditions are explored. These are shown in the following Tables 2 and 3.

TABLE 2

| | | |
|---|---|---|
| Flow Condition | Condition 1 | Conditions 2 |

SMR Conditions

| | | | | |
|---|---|---|---|---|
| Natural gas | 22 | SLPM | 22 | SLPM |
| flow rate | | | | |
| N2 flow rate | 1.5 | SLPM | 1.5 | SLPM |
| H2 flow rate | 2.9 | SLPM | 2.9 | SLPM |
| CO2 flow rate | 6.65 | SLPM | 6.65 | SLPM |
| Water flow rate | 35.9 | ml/min | 35.9 | ml/min |
| Inlet Temperature | 350° | C. | 350° | C. |
| Outlet pressure | 181.8 | psig (1.25 MPa) | 219 | psig (1.51 MPa) |

Combustion Conditions

| | | | | |
|---|---|---|---|---|
| H2 flow rate | 21.80 | SLPM | 19.46 | SLPM |
| CH4 flow rate | 0.38 | SLPM | 0.35 | SLPM |
| CO flow rate | 0.00 | | 0.76 | SLPM |
| CO2 flow rate | 0.00 | | 1.59 | SLPM |
| N2 flow rate | 3.34 | SLPM | 1.00 | SLPM |
| Air flow rate | 67.7 | SLPM | 67.3 | SLPM |
| Inlet Temperature | 372° | C. (fuel), | 372° | C. (fuel), |
| | 321° | C. (Air) | 321° | C. (Air) |
| Outlet pressure | 0.6 | psig (4.14 kPa) | 0.6 | psig (4.14 kPa) |

TABLE 3

| | Start 1 | Start 2 | | Start 3 | | Start 4 | |
|---|---|---|---|---|---|---|---|
| Performance | Condition 1 | Condition 1 | Condition 2 | Condition 1 | Condition 2 | Condition 2 | Condition 2 |
| Hours from | 163 | 858 | 912 | 1152 | 1232 | 1729 | 1867 |
| start-1 | | | | | | | |
| H₂ production | 44.2 | 46.2 | 42.7 | 48.0 | 44.3 | 46.0 | 43.9 |
| based on | | | | | | | |
| reactor with | | | | | | | |
| 640 parallel | | | | | | | |
| channels | | | | | | | |
| which is 160 | | | | | | | |
| times greater | | | | | | | |
| than | | | | | | | |
| demonstrated | | | | | | | |
| reactor (kg/h) | | | | | | | |
| CO | 201.4 | 196.8 | 183.3 | 202.3 | 190.2 | 198.2 | 187.9 |
| production | | | | | | | |
| based on | | | | | | | |
| reactor with | | | | | | | |
| 640 parallel | | | | | | | |
| channels | | | | | | | |
| which is 160 | | | | | | | |
| times greater | | | | | | | |
| than | | | | | | | |
| demonstrated | | | | | | | |
| reactor (kg/h) | | | | | | | |

Process Performance

| | | | | | | | |
|---|---|---|---|---|---|---|---|
| CH4 | 77.6% | 77.1% | 76.3% | 77.5% | 76.4% | 75.6% | 75.3% |
| Conversion | | | | | | | |
| CO Selectivity | 70.4% | 71.5% | 70.0% | 72.1% | 69.8% | 70.7% | 70.1% |
| DP, psi | 12.6 | 12.0 | 10.0 | 12.1 | 10.1 | 10.1 | 10.3 |
| (kPa) | (86.9) | (82.7) | (68.9) | (83.5) | (69.6) | (69.6) | (71.0) |

Combustion Performance

| | | | | | | | |
|---|---|---|---|---|---|---|---|
| CH4 | 100% | 100% | 100% | 100% | 100% | 100% | 100% |
| Conversion | | | | | | | |
| H2 | 99.9% | 100% | 99.9% | 99.9% | 100% | 100% | 100% |
| Conversion | | | | | | | |
| Fuel DP, psi | 38.9 | 39.1 | 32.1 | 38.2 | 32.6 | 32.9 | 33.0 |
| (kPa) | (268) | (270) | (221) | (263) | (225) | (227) | (228) |

Reactor Temperature

| | | | | | | | |
|---|---|---|---|---|---|---|---|
| Maximum | 972 | 971 | 970 | 973 | 973 | 976 | 975 |
| reaction | | | | | | | |
| temperature, | | | | | | | |
| ° C. | | | | | | | |
| U-Turn | 908 | 912 | 914 | 912 | 920 | 923 | 922 |
| temperature, | | | | | | | |
| ° C. | | | | | | | |
| Total heat | 3008.4 | 2950.8 | 2882.6 | 3036.4 | 2966.1 | 3010.2 | 2895.5 |
| transferred | | | | | | | |
| for | | | | | | | |
| demonstrated | | | | | | | |
| 4 parallel | | | | | | | |
| channel | | | | | | | |
| reactor (W) | | | | | | | |
| Heat | 2661.0 | 2630.6 | 2554.9 | 2716.1 | 2649.0 | 2729.9 | 2606.4 |
| transferred in | | | | | | | |
| the reaction | | | | | | | |
| section for | | | | | | | |
| demonstrated | | | | | | | |
| 4 parallel | | | | | | | |
| channel | | | | | | | |
| reactor (W) | | | | | | | |
| Average heat | 38.7 | 38.2 | 37.1 | 39.5 | 38.5 | 39.7 | 37.9 |
| flux in the | | | | | | | |
| reaction | | | | | | | |
| section for | | | | | | | |
| demonstrated | | | | | | | |
| 4 parallel | | | | | | | |
| channel | | | | | | | |
| reactor | | | | | | | |
| (W/cm²) | | | | | | | |
| Overall | 25.5 | 25.0 | 24.4 | 25.7 | 25.1 | 25.5 | 24.5 |
| average heat | | | | | | | |
| flux (W/cm²) | | | | | | | |
| Equilibrium | 92.0% | 92.6% | 91.0% | 92.4% | 91.8% | 92.1% | 91.9% |
| conversion | | | | | | | |
| Equilibrium | 77.3% | 77.6% | 77.1% | 77.9% | 77.5% | 77.8% | 77.7% |
| selectivity | | | | | | | |
| Equilibrium | 834.0 | 831.7 | 840.8 | 833.3 | 841.6 | 838.9 | 838.7 |
| Temperature | | | | | | | |
| based on | | | | | | | |
| CH₄ | | | | | | | |
| conversion, | | | | | | | |
| ° C. | | | | | | | |
| Equilibrium | 842.9 | 851.2 | 849.4 | 853.8 | 847.9 | 854.9 | 851.4 |
| Temperature | | | | | | | |
| based on CO | | | | | | | |
| selectivity, ° C. | | | | | | | |

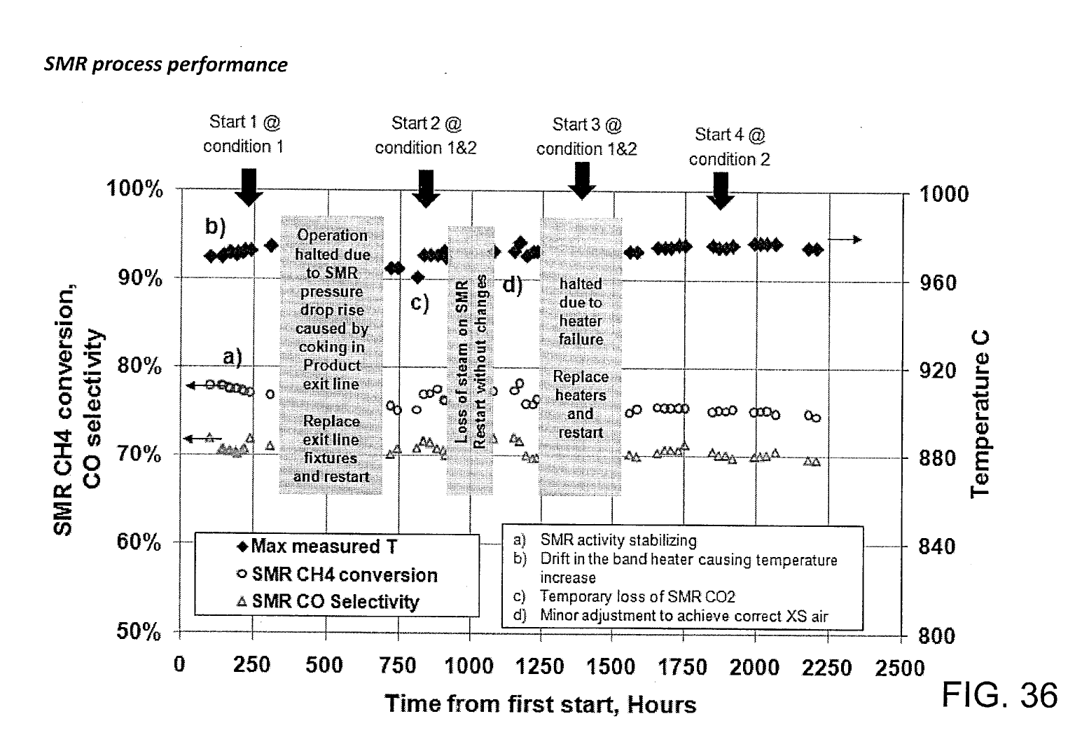

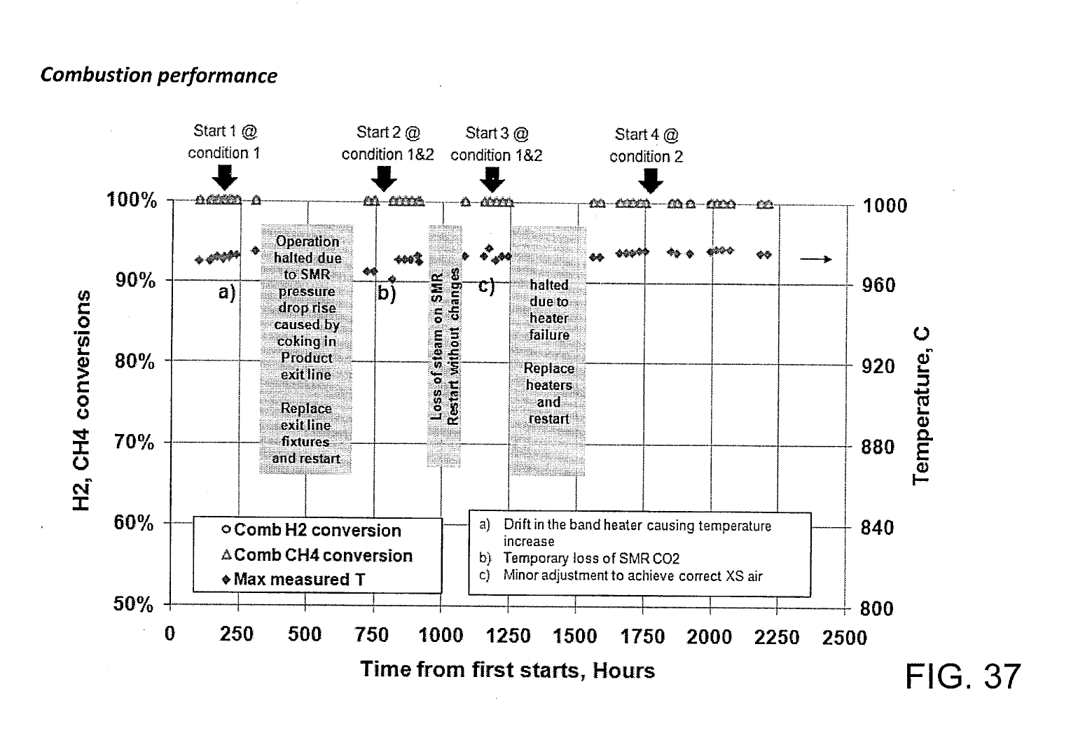

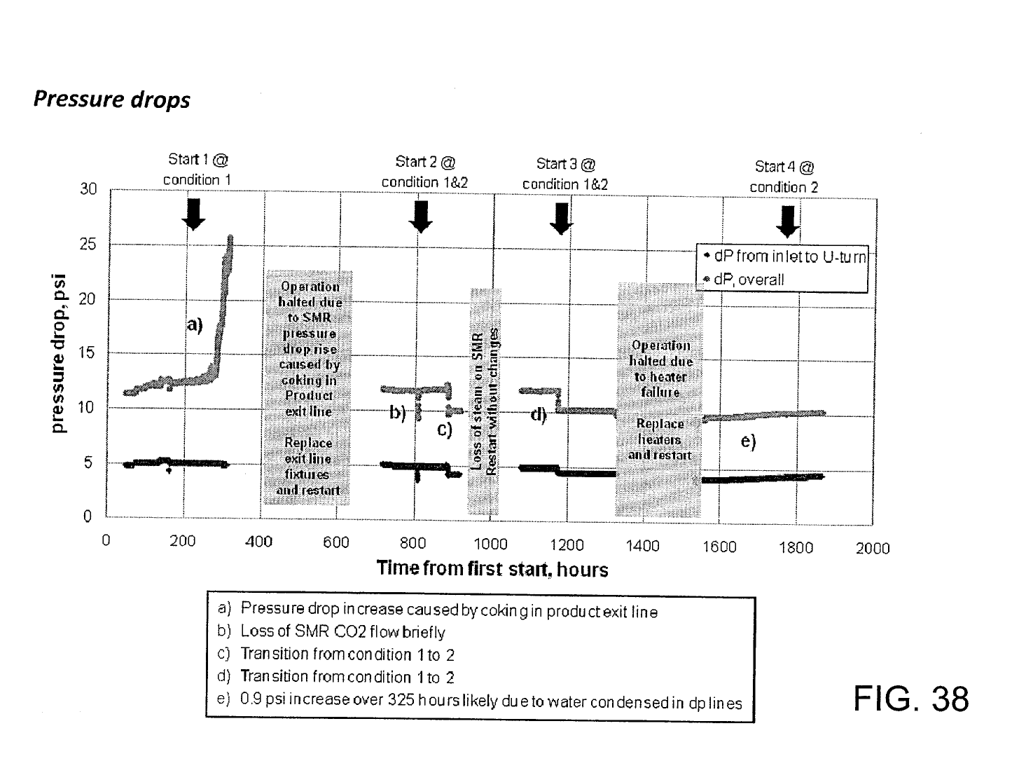

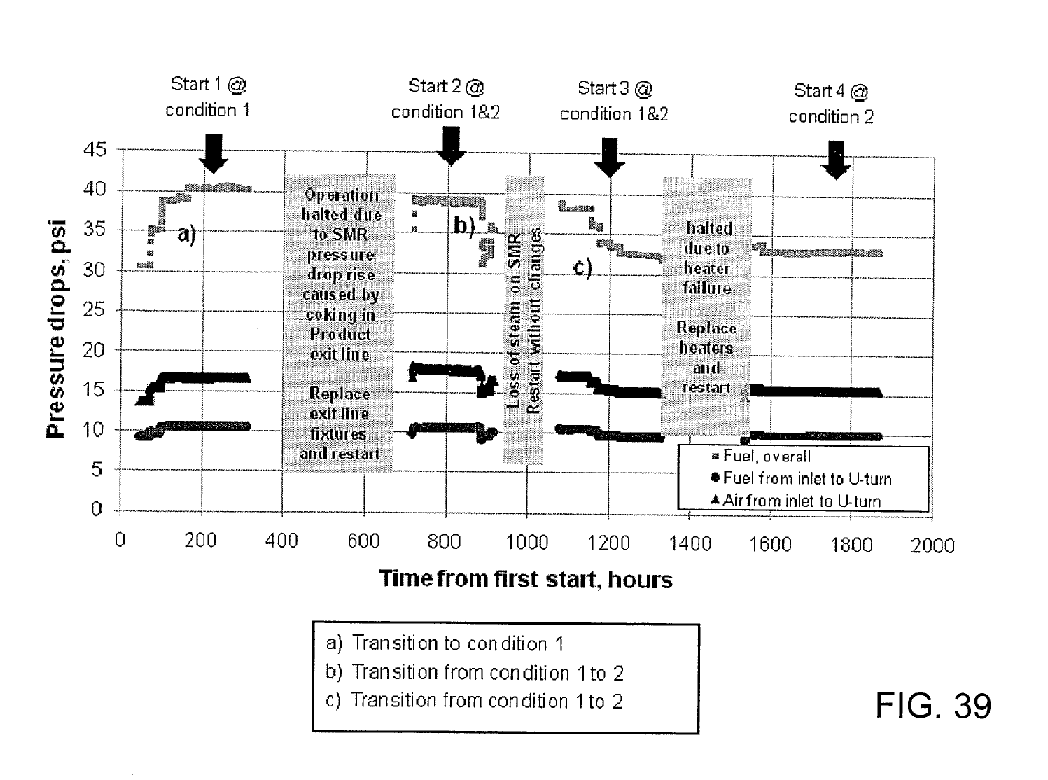

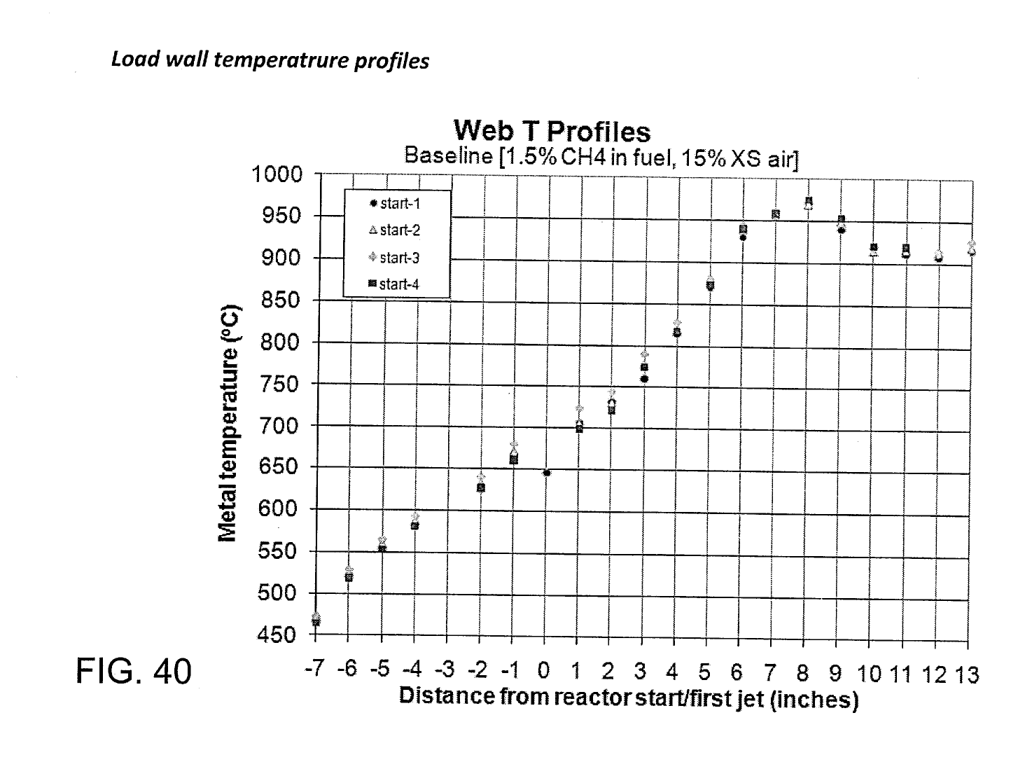

There are several reactor starts and restarts after process upsets. These are shown in Table 3. Start 2 occurs after a pressure drop rise in a downstream fitting. After shutdown, the fitting, which is a stainless steel fitting, is replaced with an Inconel fitting prior to restarting the operation. Start 3 occurs after a loss in the process water inlet—where no steam is fed to the SMR for about 2 minutes. During this upset, the peak recorded temperature on the reactor load wall increases to 1065° C. before the system interlocks. At interlock, the peak temperature drops about 200° C. in 40 seconds before a more gradual cool down commences. Start 4 occurs after several external heaters are added to reduce thermal losses. For all cases, the reactor performance comes back to equivalent and target performance. The results are shown in FIGS. 36 to 40.

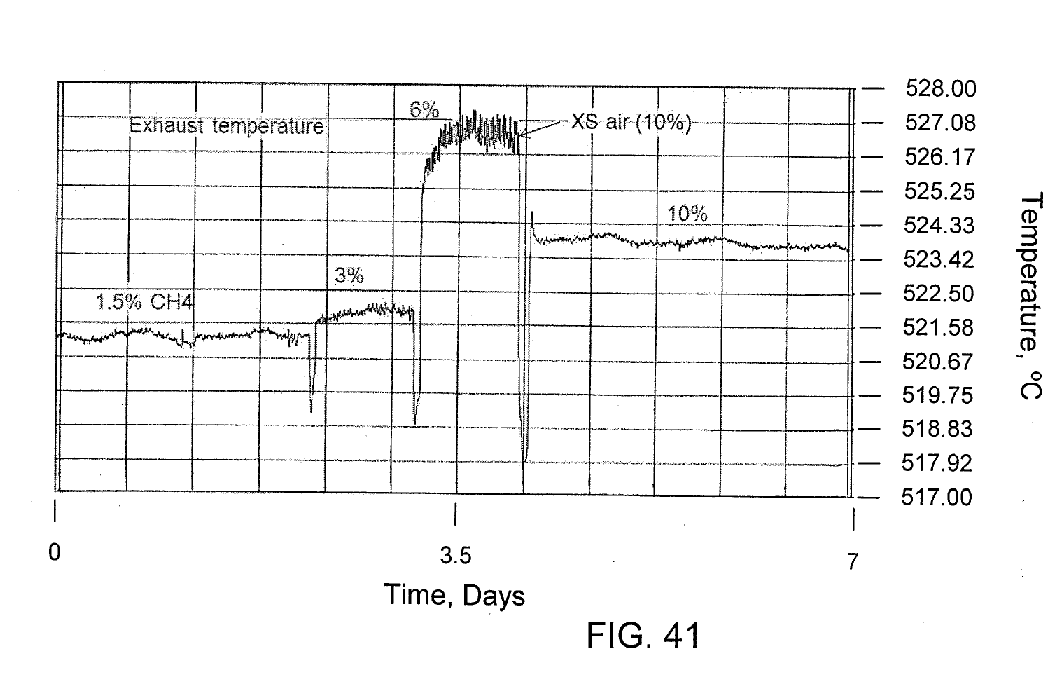

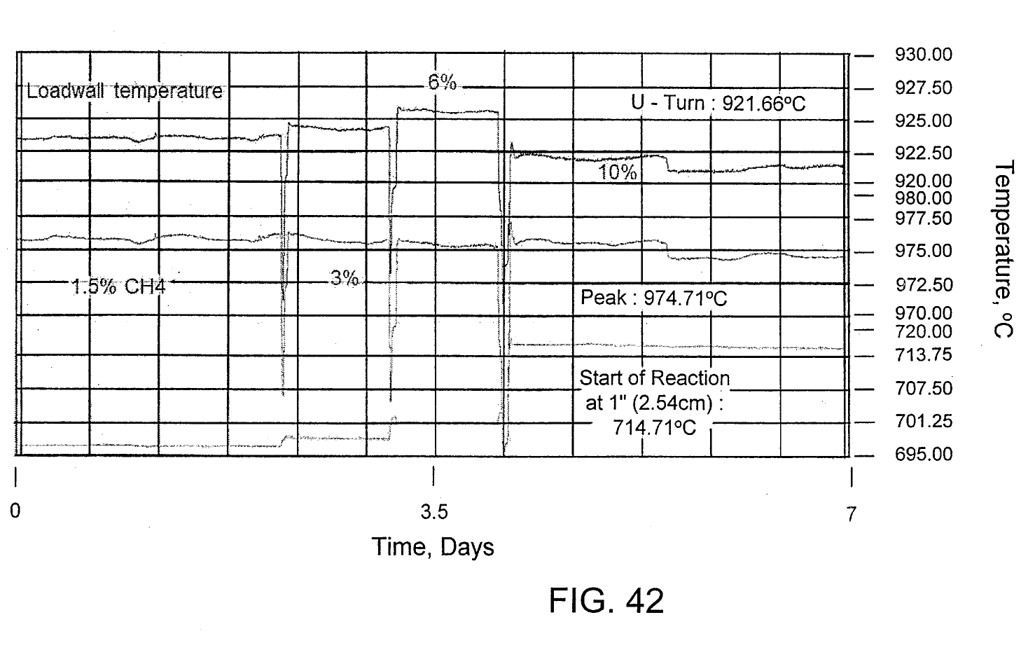

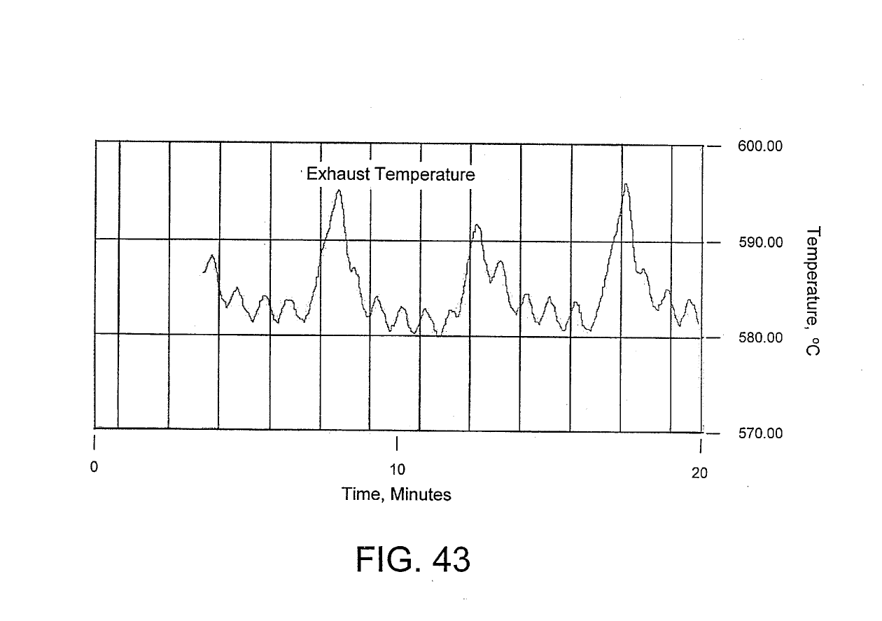

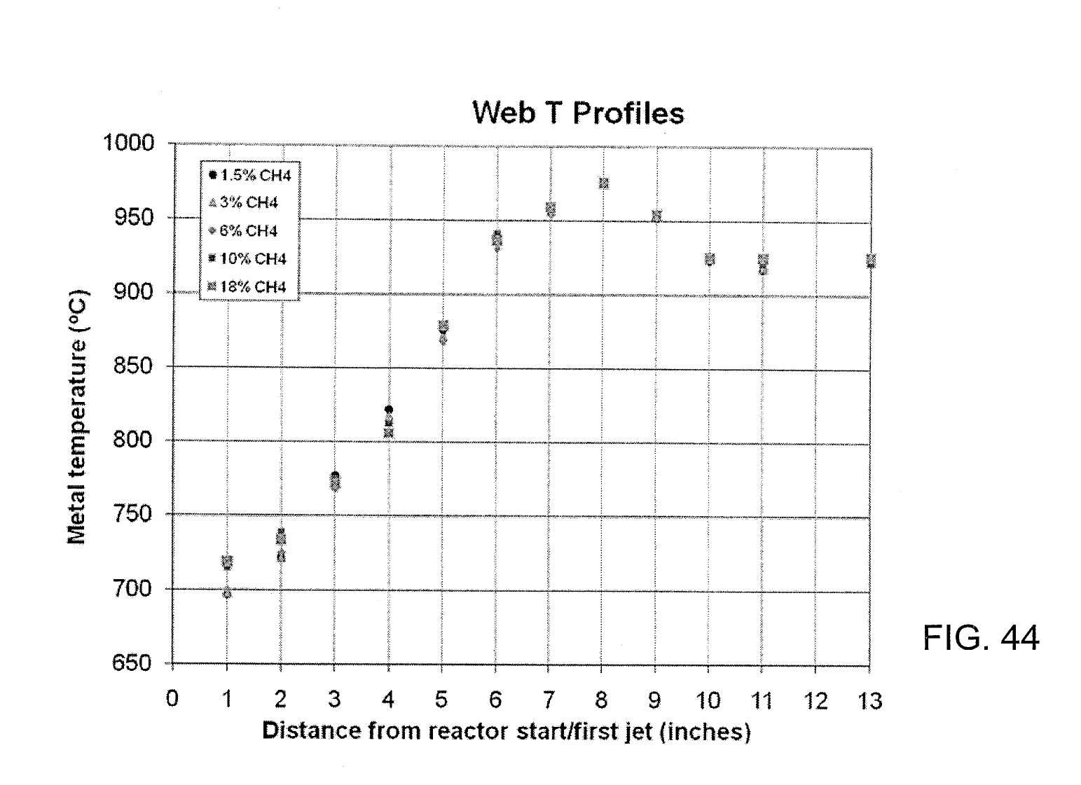

Additional test runs are conducted with the reactor, including the addition of more methane to the combustion fuel. Methane is significantly more challenging to combust than hydrogen. In condition 1 and 2 as reported, there is 1.5% methane by is volume in the fuel. The amount of methane in the combustion fuel increases to 18% and there are no detectable methane emissions across this range (1.5%, 3%, 6%, 10% and 18%). The detection limit is roughly 100 ppm methane. The nominal amount of excess air for all cases is 15%, but in some test runs the amount of excess air is lower. There is some instability in outlet exhaust temperature at 6% methane fuel when the excess air is lowered to 10%. The results are shown in FIGS. 41 to 44.

### Example 3

### Ex Situ Catalyst Coating in a Welded SMR Reactor

A SMR reactor has two types of catalysts: 1) catalyst to combust fuel that provides energy for the SMR reaction, and 2) catalyst for the SMR reaction. The catalysts are preferentially coated on only portions of the wall of the microchannel at pre-determined locations for the reactions to occur.

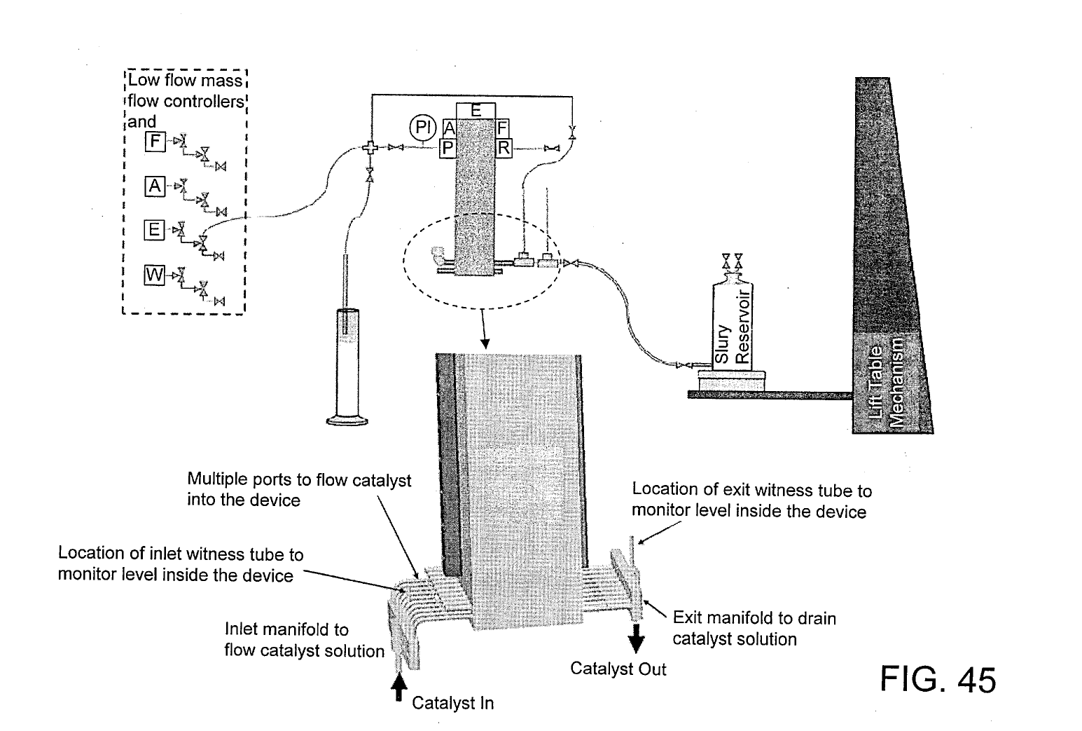

The manufacture of SMR reactors using diffusion bonding involves the bonding of shims and plates at very high temperatures (e.g., in excess of about 1000° C.). As a result of these high temperatures, the catalyst is applied only after the reactor core is diffusion bonded. However, after the reactor core is diffusion bonded, there is no visual access to the microchannels and the catalyst is applied to the walls of the microchannel using fill and drain techniques, whereby the microchannels are filled with a catalyst solution or slurry and then drained, the draining being assisted by gravity. This may be referred to as an in-situ process or approach. It may also be referred to as in-situ washcoating. This in-situ approach of applying catalyst to the walls of the microchannels has the following disadvantages:
1. Typically multiple fill and drain cycles were required to apply catalyst coating to the wall.
2. The catalyst loading on the walls were generally low (˜5 to 10 mg/in² after 4 fill and drain cycles).
3. Since there was no visual access to microchannel, the method had less control on the catalyst flow inside the microchannels. It was difficult to selectively apply the catalyst to specific axial or lateral locations. It was also not possible to create an axially discontinuous coating, whereby a catalyst would be added for one part of the reaction channel length followed by an intermittent region with no is catalyst then followed by a third region with a catalyst.
4. In-situ wash-coating is a slow process. Even single microchannel devices could require up to one week for catalyst coating. Coating catalysts on commercial scale devices (>100 kg/hr process flow rate) required complex additional manifolds for coating. FIG. 45 shows a schematic of a set-up for coating a SMR reactor with multiple microchannels.
5. The catalyst could not be easily maintained at a specific height in the reactor because of capillary forces that wicked the solution to a higher location especially in the device corners or crevices.
6. The in-situ method of applying catalyst requires a large volume of catalyst to coat a small area. Due to the use of the manifold system for filling and draining the catalyst solution, a large volume of the catalyst solution was required initially. However, only a small portion of this catalyst solution actually remained in the reactor. The catalyst solution that drained out of the reactor then had limited uses and often had to be disposed of or recycled after only one or two uses.

The welded approach for manufacturing SMR reactors pursuant to the invention allows for a simple, fast and accurate coating method for of the catalyst. The welded approach may be used to replace high temperature diffusion bonding of multiple shims by welding of fewer plates. The high temperature required for welding may be localized at the edges of the plates and does not affect the microchannels where catalyst needs to be applied. Therefore, the catalyst may be applied ex-situ prior to the welding of the plates.

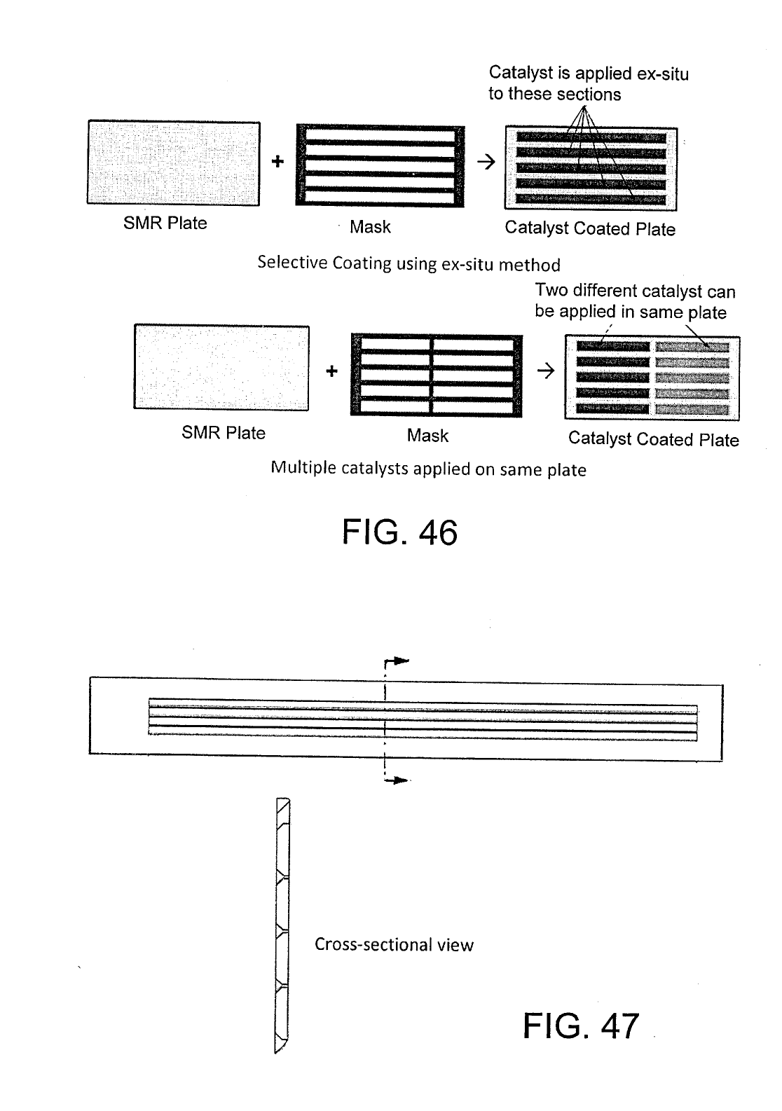

With the ex-situ method of applying the catalyst, the catalyst solution may be applied using simple methods such as the use of an air jet through an airbrush. Since there is full visual access to the microchannels, the locations where catalyst is not required can be masked off easily as shown in FIG. 46. Also, different catalyst can be applied at specific locations within the same microchannel to achieve good performance. The coating coverage level may be determined using reference coupons which are weighed before and after coating to determine the amount of catalyst coated.

After the catalyst is applied, the plates may be dried in air prior to welding to build the SMR reactor. The SMR reactor may then calcined at about 450° C. to form the final catalyst on the walls of the microchannels.

The ex-situ catalyst coating method has several advantages over the prior in-situ catalyst coating method. These may include:
1. The ex-situ technique is significantly faster than the in-situ coating technique. A reactor that may typically take about one week for in-situ catalyst coating, can be coated within a day using the ex-situ method.
2. Ex-situ coating enables control over location, type and quantity of the catalyst applied.
3. A good reproducibility of catalyst loading levels can be achieved using ex-situ coating methods.
4. Coatings other than catalysts may also be added to plates either before or after the catalyst is coated or on plates in the assembly that do not contain catalysts.
5. The ex-situ coating allows for a smaller volume of catalyst solution to be prepared due to the ability to control the location of application, so less catalyst solution is wasted.

A multichannel SMR reactor is designed, manufactured and tested for performance. The combustion and the SMR catalysts are applied to the plates using the ex-situ method. The combustion catalyst is applied to Cat-plate (facing fuel channel) and A-E plate (exhaust channel). The process catalyst is applied to Cat plate (facing reactant channel) and R-P plate (reactant channel).

For the catalyst application, a slurry is prepared comprising the desired catalyst for the plate being coated. A masking plate, which is shown in FIG. 47, is used. A cross-sectional view of the masking plate is also shown in FIG. 47. The masking plate is made out of carbon steel, although it could also be made from any hard or flexible material. The mask is designed to coat the four process channels in the multichannel reactor. The cross-sectional area of each channel to be coated with catalyst is 0.16 by 13 inches (0.41 by 33.0 cm). The regions outside the masking plate are masked using construction tape.

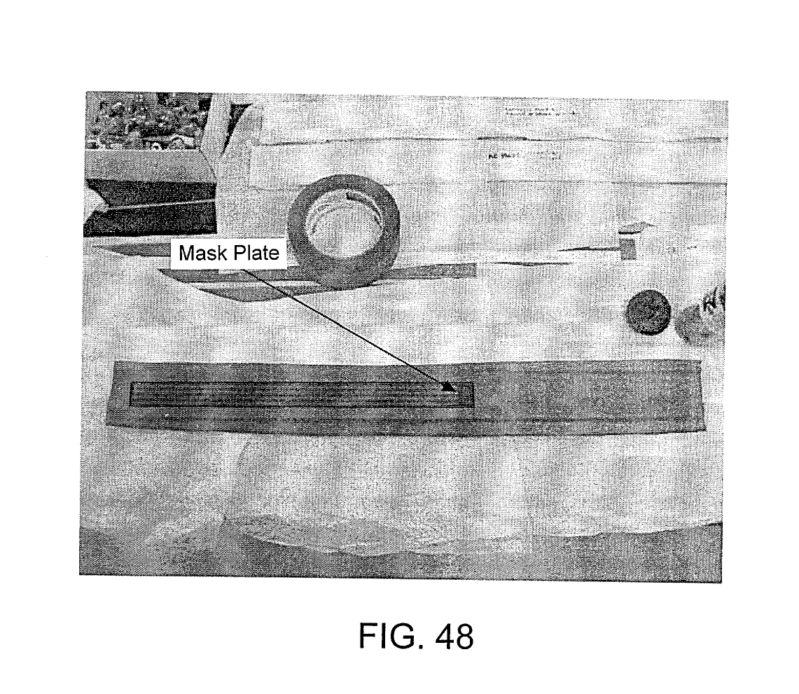

The catalyst solution is applied using a Paasche Airbrush Set, single-action, siphon feed, external mix, using 32-35 psi (0.22-0.24 MPa) pressure for spraying of slurries, using a #1 nozzle set up. FIG. 48 shows a picture of masked plate after coating. The catalyst loading on the R-P plate is 25 mg/in² (3.87 mg/cm²).

### Example 4

### Addition of a Coating or Layer to Resist Metal Dusting in SMR Reactors

Alloys based on iron, nickel or cobalt may be susceptible to metal dusting corrosion in the presence of carbon monoxide (CO) gas. Although efforts have been made to develop new metal alloys that are more resistant to metal dusting corrosion, there are currently no commercially available alloys that are immune to metal dusting corrosion. There is a need to develop a coating to protect the alloy from metal dusting corrosion. The alloy used for this example is Inconel 617 (an alloy containing Ni, Cr, Fe, Mo, Al and Co), although the problem of metal dusting may occur on any nickel or iron bearing metal or metal alloy.

When metal dusting starts, the resultant pits may eat away through the pressure boundary of a channel. Further, the pits may be more likely to lead to the onset of coking through the Boudouard reaction of CO+CO to C(s) and CO₂. As coke is initiated, it continues to grow, typically in a filamentous form that may fully or partially block a microchannel. Channel blockage may lead to flow maldistribution in a multichannel device, a reduction in performance, and higher pressure drops.

The coating may be used to prevent gas molecules such as CO from reaching the metal alloy. The coating itself may not metal dust and may be compatible with the environment of use.

The coating may comprise a single layered coating. The coating material may comprise a ceramic, such as alumina.

The coating should be free of defects such as pinholes or micro-cracks to prevent gas molecules from reaching the alloy underneath. The coating may be hermetic. Ceramics are brittle in general and are prone to cracking. Metals are in general more ductile than ceramics, thus less prone to cracking. The metallic coatings may include copper, chromium, silver, gold, mixtures of two or more thereof, as well as other inert or noble metals. Problems may be associated with the use of metal coatings. One problem may be that inter-diffusion may occur between the metal coating and the substrate alloy. Metal dusting may occur in a temperature range of about 450° C. to about 750° C. In this temperature range, inter-diffusion between the metallic coating and the alloy may be expected. Over time, Ni, Co and Fe may diffuse out from the alloy to the coating, making the coating less resistant or protective. Inward diffusion of the coating material into the alloy may also cause undesirable changes to the properties of the alloy. Another problem relates to making the coating free of defects such as pinholes. Although it is difficult to produce a defect-free coating, increasing the coating thickness in general may reduce the population density of defects like pinholes.

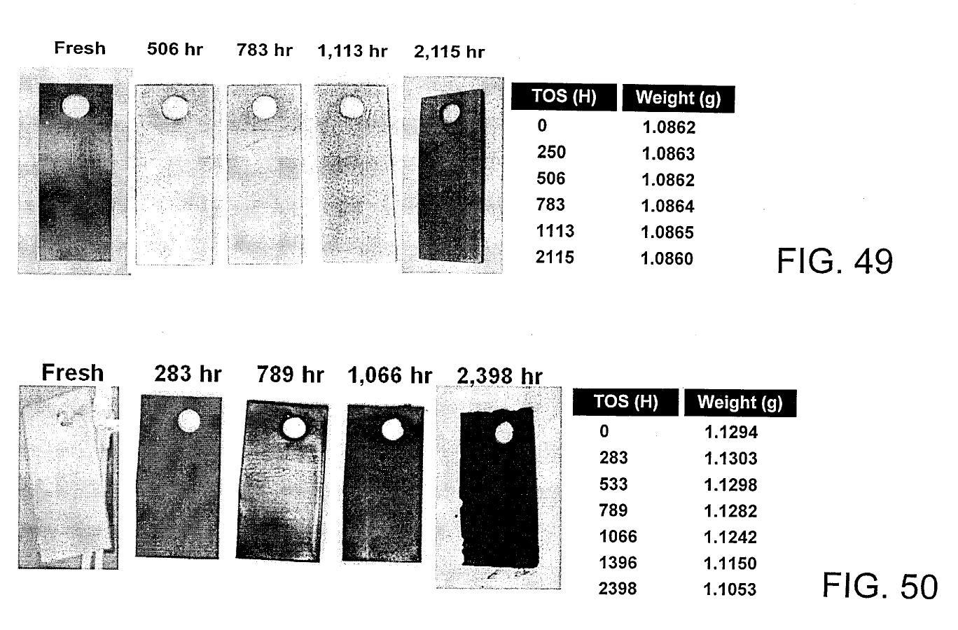

FIG. 49 shows a copper-coated Inconel 617 coupon after exposure to a metal dusting environment for various durations of time. The coupon gradually loses its bright copper appearance, but no metal dusting corrosion occurs. Also, there are no measurable weight changes after 2,000 hours on stream. This is shown in FIG. 49. By comparison, the uncoated Inconel 617 coupon is visibly pitted at 1000 hours, and is severely corroded at 2400 hours on stream. This is shown in FIG. 50. The weight losses shown in FIG. 50 are additional evidences of corrosion.

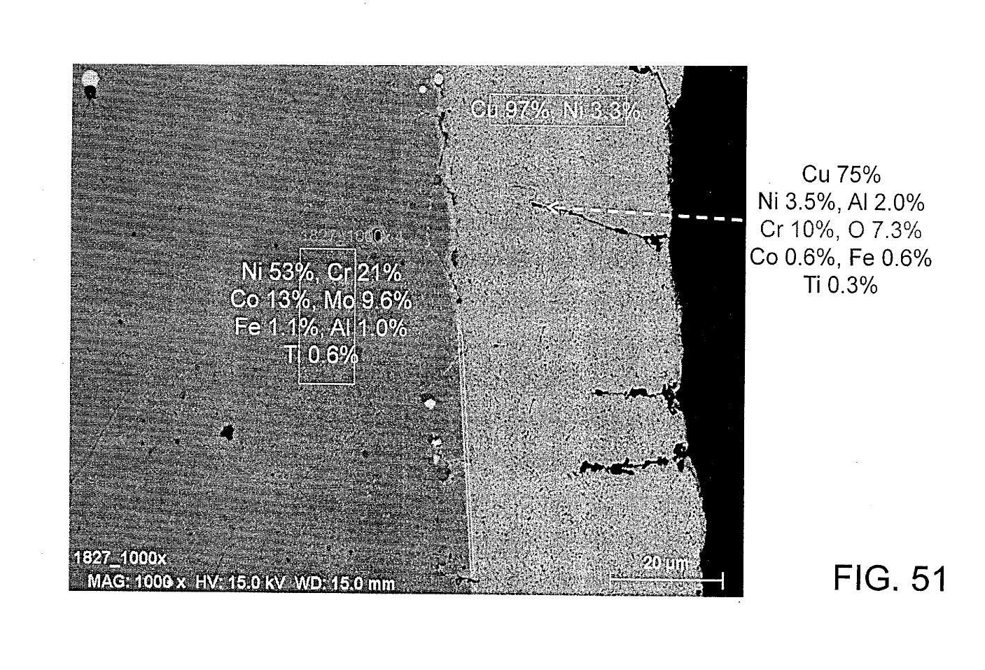

Cross section analysis of a copper-coated coupon after 863 hours of exposure shows Ni diffusion into the Cu coating and the development of micro-cracks in the coating. This is shown in FIG. 51. This indicates that copper may be a protective coating against metal dusting for the short term.

To prevent inter-diffusion between the coating and the substrate, a diffusion barrier may be used. A ceramic coating such as alumina may be a good barrier as metals typically do not diffuse through ceramic.

A two-layer coating system may work better than a single layer coating for metal dusting resistance. The first layer may comprise a diffusion barrier, for example, a ceramic coating layer such as an alumina coating layer. The alumina coating layer may be deposited directly on the substrate or formed as a thermally grown alumina scale from heat treating an aluminum containing metal alloy. Some alloys commercially available are alumina formers. Examples of such aluminum containing metal alloys may include Inconel 693 (an alloy containing nickel, chromium and aluminum) and Haynes 214 (an alloy containing nickel, chromium, aluminum and iron). For other alloys, aluminization may convert the surface of the alloy to aluminide as a diffusion coating. An alumina scale may then be thermally grown by heat treating the aluminized alloy.

The second layer may comprise a metal coating that is ductile and covering. The materials that may be used may include Cu, Cr, Al, Ag, Au, mixtures of two or more thereof, as well as other metals not prone to metal dusting, for example, metal carbides. These may include composites of two or more metals as either an alloy, or a bi-layer, or a tri-layer coating.

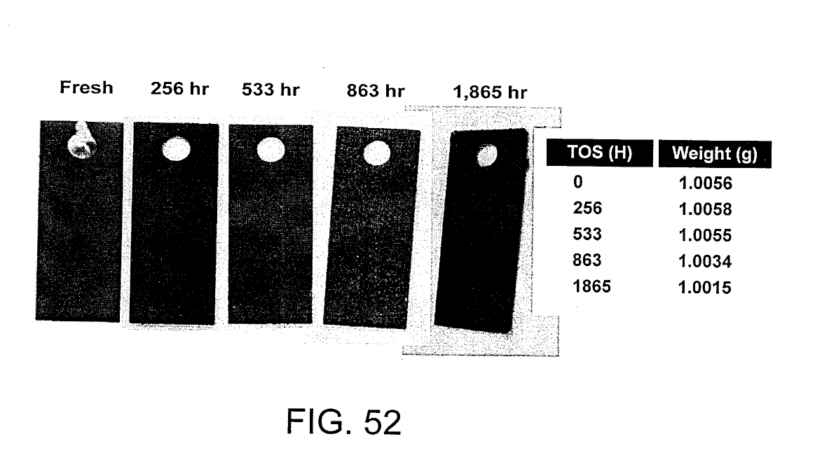

The second layer may comprise a ceramic coating, making the coating system totally ceramic. Although ceramic coatings may be prone to cracking, using two layers may reduce the likelihood of having cracks lined up in both coatings with the substrate alloy underneath exposed. FIG. 52 shows the performance of such a two-layer ceramic coating of titanium carbide on alumina using an Inconel 617 coupon. Although there are small weight losses, as shown in FIG. 52, the coated coupon performs better than the uncoated coupon shown in FIG. 50.

The second layer may comprise an alloy coating that is still ductile but having a better matching CTE (coefficient of thermal expansion) with the substrate. Examples may include Al—Cu alloys, Al—Ag alloys, Al—Cr alloys, Cu—Cr alloys, and the like. An additional benefit of using an aluminum-containing alloy as the second layer relates the possibility of it forming an alumina scale at the surface, whether by a dedicated heat treatment prior to use or by natural formation during use.

With the formation of an alumina scale on top of the aluminum containing coating, the coating system becomes a three-layer system. An increase in the number of layers may decrease the likelihood of having pinholes lined up through all layers to cause the undesirable exposure of the substrate alloy underneath. An alumina coating may also be deposited directly on the metallic coating. Alumina deposition can be done by using either physical vapor deposition (PVD) or chemical vapor deposition (CVD).

Further increasing the number of layers may be beneficial. As an example, coupons of Inconel 617 may be aluminized and heat treated to generate a thermally grown alumina scale. The alumina scale may have a thickness of about 0.5 to about 1.0 micron. The coupons may then be coated with a layer of aluminum bronze by cathodic arc deposition. Two thicknesses of aluminum bronze coating are tested. One is 20 microns thick and the other is 40 microns thick. The coupons are treated in hydrogen at 950° C. for 4 hours. After the treatment, the surfaces of the coupons are covered by a top layer of alumina. These coupons are thermal cycled 12 times between 100° C. and 850° C. Each coupon shows no indication of coating loss or damage such as cracking, spallation or flaking.

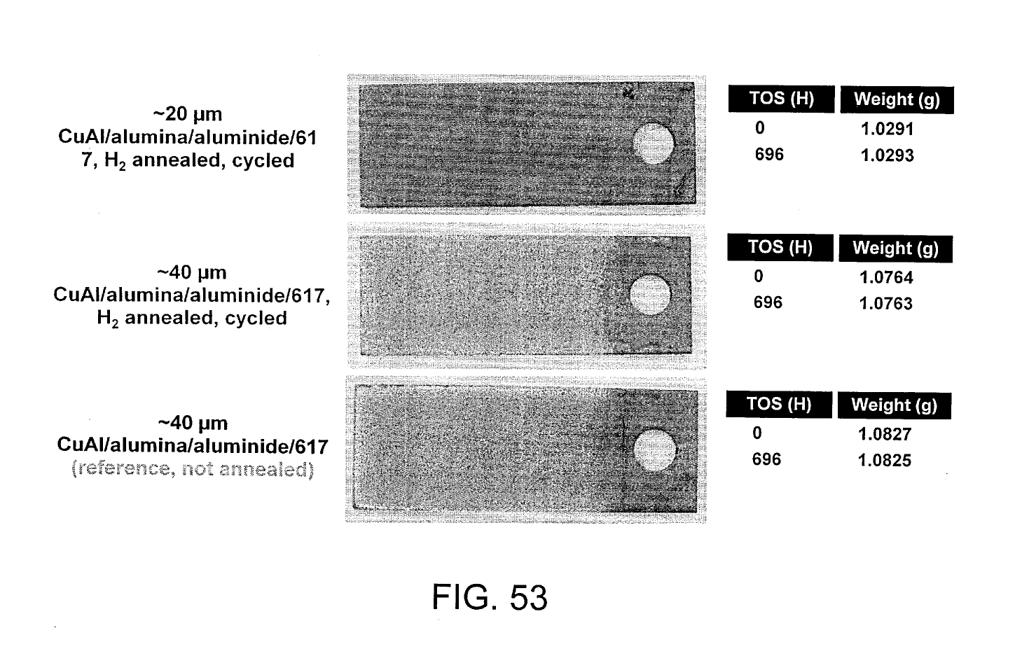

Coupons are then tested for metal dusting resistance together with is unprotected coupons. Test conditions are harsh, at a pressure of 380 psig (1.62 MPa) and a temperature of 620° C. The gas environment contains 58.4% H₂, 18.4% CO, 12.3% CO₂, 6.1% N₂ and 4.9% CH₄. The absence of water vapor in the gas environment makes the test exceptionally aggressive. After 700 hours of testing, no aluminum bronze coated coupons show visible failure or weight loss. This is shown in FIG. 53. By comparison, a SS304 coupon is severely corroded in just 250 hours. Pitting of un-protected Inconel 617 occurs between 100 and 1,000 hours.

Effective protection against metal dusting may include a series of steps:

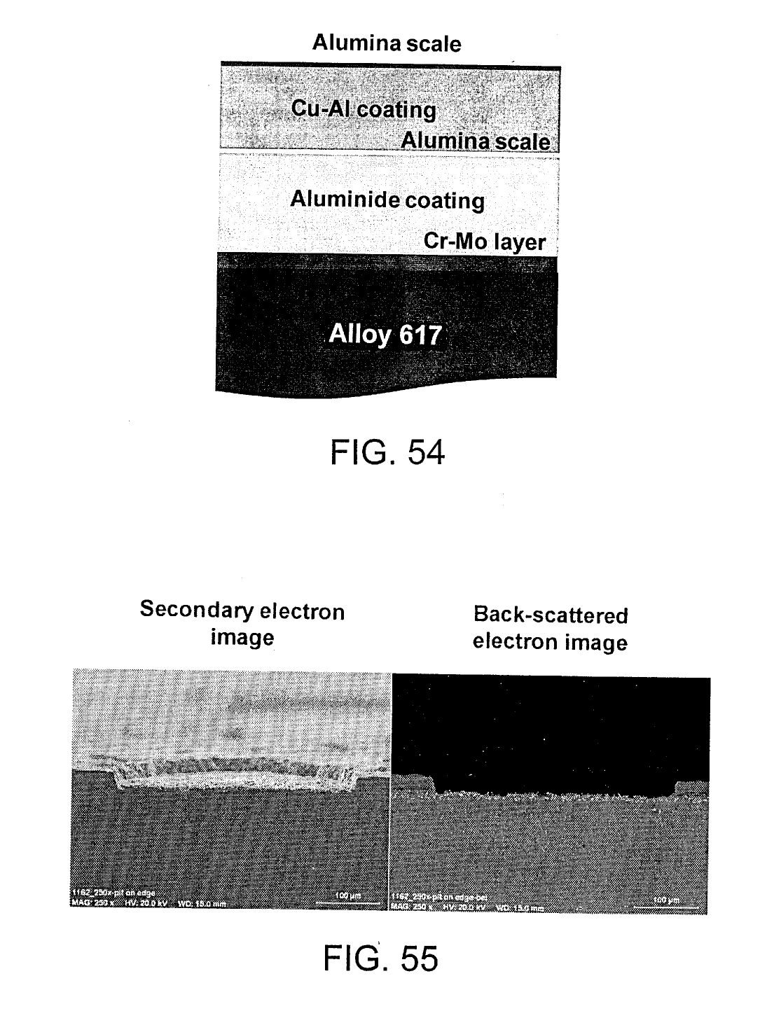

Step 1: A first alumina scale that addresses the CO-containing gas stream may provide a first line of defense against gas ingress toward the metal if there are cracks in the alumina scale
Step 2: A carburizing resistant coating, such as a Cu—Al alloy, which is not inherently attacked by CO may comprise a second line of defense against CO ingress toward the metal if there are cracks in the coating.
Step 3: A second alumina scale that provides a third line of defense against gas ingress toward the metal, if there are cracks in the alumina scale.
Step 4: A Cr—Mo interdiffusional layer, which may be formed from the aluminization process, may enhance resistance toward metal dusting. This is shown in FIGS. 54 and 55. FIG. 55 shows where metal attack stopped in this zone.
Step 5: Product design with interconnected channels which contain the CO-bearing stream. If the first four lines of defense fail and coking resultant from pitting occurs, then the gas may redistribute throughout the device to keep the reactor in service.
Step 6: Refurbishment—if carbon build up occurs over time and the redistribution is no longer effective, then the welded plates can be taken apart and the coke removed from the surface. An additional barrier coating may be placed over the pitted zone to put the plate back in service.
Step 7: Replacement—if the plate that contains metal dusting cannot be repaired, then the particular plate can be replaced with a fresh plate when the full reactor is put back into service—thus sacrificing a part to save the whole.

The metal dusting resistant coating may be selectively coated in the reactor locations which are designed to operate at temperatures which are susceptible to metal dusting (e.g., from about 450 to about 750° C.). The inventive reactor technology allows for the use of masks or other means to occlude a coating from is higher or lower temperature regions or from channels which may process fluids that do not create metal dusting.

### Example 5

### Refurbishment of Catalyst Coating

The SMR and combustion catalysts may be expected to deactivate over time. Also, undesirable conditions such as coke formation due to unsuitable operational conditions may cause partial or complete plugging of the microchannels leading to inadequate performance. It would be advantageous if the SMR reactor had the ability to refurbish the catalyst coating or remove unwanted deposits under such circumstances. There is no straightforward way to remove the coated catalyst from the inside of bonded microchannels.

The welded manufacturing approach provided by the present invention allows for disassembly of the SMR reactor into individual plates, thus giving the same access to all of the plates as available prior to weldment of the reactor. The steps to refurbish the catalyst in the SMR reactor may be as follows:

1. Disassembly of the Reactor into Individual Plates
The weld around the plates and manifolds may be removed to release the plates. Methods such as conventional grinding and machining may be used to remove the welds. After the plates are released, they are inspected for any deformation. If the plates are deformed, they may be either remediated with a thermal annealing step of mechanical flattening, or they may be replaced with new plates.

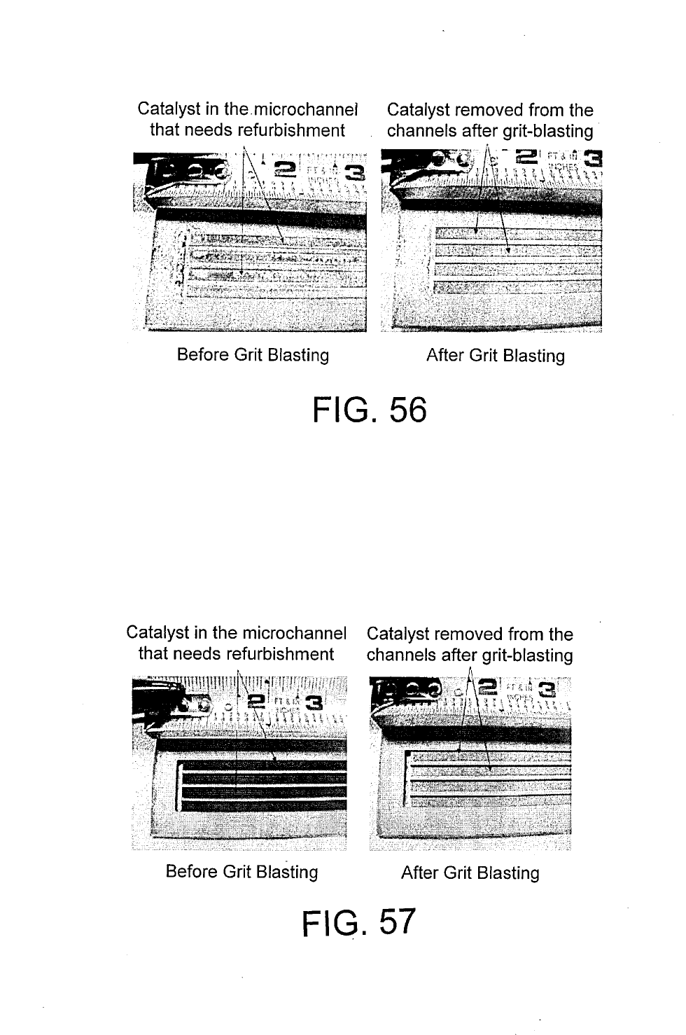

2. Removal of Catalyst from the Plates
The locations to remove the catalyst are identified. The locations may be preferentially grit blasted with high-purity white alumina particles (220 grit size). The intensity of the alumina particles may be adjusted such that only the catalyst is removed. Other size grit or materials may be used for removing catalyst from the walls. Alternative methods for removing spent catalyst from the walls may include sonication and mechanical agitation. FIG. 56 shows a comparison of before and after grit blasting of a Cat-plate. FIG. 57 shows a comparison of before and after grit blasting of a R-P-plate.

3. Heat Treatment (Optional)
If the alumina scale on the plates is damaged, the plates may be heat treated is to replenish the alumina scale. An example of a heat treatment method may include:
   a. Heat the plates in controlled environment of 18 ppm O₂ in Ar from ambient temperature to 1050° C.
   b. Heat treat the plates in 21% O₂ (by mole) in Ar for 10 hours at 1050° C.
   c. Cool the plates to ambient temperature in 21% O₂ (by mole) in Ar.
Alternatively, the plates may be heated in an open box furnace or with an alternate combination of diluted or undiluted air.

4. Apply Catalyst
Apply the catalyst using the same methods as before. Masks may be used on the plates to apply catalyst on the desired locations only. After the catalyst is applied, it may be dried in air.

5. Weld Plates
The plates may be welded together using the same manufacturing steps as discussed above. The core may be welded first followed by attachment of manifold and inlet/outlet tube connections.

6. Activate the Catalyst and Operate the Reactor
The reactor may be installed in a facility where the catalyst may be activated. The reactor may then be ready to operate.

### Example 6

A SMR reaction is conducted using two separate reactors. The first reactor, which is referred to as a “Welded” reactor, is made using peripheral welding and ex-situ catalyst coating pursuant to the present invention. The other reactor, which may be referred to as a “Bonded” reactor, is made using diffusion bonding and in-situ catalyst coating. The results are shown in the following Table 4.

TABLE 4

| | | Reactor | |
|---|---|---|---|
| Performance Parameter | Units | Welded | Bonded |
| Inlet operating pressure | psig (MPa) | 231 (1.59) | 320 (2.21) |
| (as designed) | | | |
| Total volumetric flow rate | SLPM | 12563 | 8315 |
| Contact time | | | |
| Catalyst channel only | ms | 4.17 | 3.05 |
| Reaction section only | ms | 8.33 | 4.83 |
| Reactor Core | ms | 14.73 | 9.53 |
| Process methane conversion | % | 76.1 | 77.8 |
| Process Peak Temperature | ° C. | 911 | 865 |
| (centerline) | | | |
| Process pressure drop | psi (kPa) | 16.2 (112) | 36 (248) |
| Total heat transfer in Reactor | kW | 523 | 259 |
| section | | | |
| Pressure drop per unit contact | kPa/ms | 26.8 | 81.3 |
| time (Catalyst section) | | | |
| Pressure drop per unit contact | kPa/ms | 13.4 | 51.4 |
| time (Reactor section) | | | |
| Pressure drop per unit contact | kPa/ms | 7.6 | 26.0 |
| time (Reactor Core) | | | |
| Total reaction heat per unit | kW/ms | 125.5 | 84.9 |
| contact time (Catalyst section) | | | |
| Total reaction heat per unit | kW/ms | 62.8 | 53.6 |
| contact time (Reactor section) | | | |
| Total reaction heat per unit | kW/ms | 35.5 | 27.2 |
| contact time (Reactor Core) | | | |
| Total reaction heat per unit | W/Pa | 4.7 | 1.0 |
| pressure drop | | | |

While the invention has been explained in relation to various embodiments, it is to be understood that various modifications thereof will become apparent to those skilled in the art upon reading the specification. Therefore, it is to be understood that the invention disclosed herein is intended to cover such modifications as fall within the scope of the appended claims.

1. An apparatus, comprising: a plurality of plates in a stack defining at least one process layer and at least one heat exchange layer, each plate having a peripheral edge, the peripheral edge of each plate being welded to the peripheral edge of the next adjacent plate to provide a perimeter seal for the stack, the ratio of the average surface area of each of the adjacent plates to the average penetration of the weld between the adjacent plates being at least about 100 cm²/mm.

2. An apparatus, comprising: a plurality of plates in a stack defining at least one process layer and at least one heat exchange layer, each plate having a peripheral edge, the peripheral edge of each plate being welded to the peripheral edge of the next adjacent plate to provide a perimeter seal for the stack, the process layer containing a steam methane reforming catalyst, the heat exchange layer containing a combustion catalyst.

3. The apparatus of claim 1 wherein the stack is positioned in a containment vessel, the stack being adapted to operate at an internal pressure above atmospheric pressure, the containment vessel being adapted to operate at an internal pressure above atmospheric pressure and provide for the application of pressure to the exterior surface of the stack, the containment vessel including a control mechanism to maintain a pressure within the containment vessel at least as high as the internal pressure within the stack.

4. The apparatus of claim 3 wherein the control mechanism comprises a check valve and/or a pressure regulator.

5. The apparatus of claim 3 wherein a reactant gas is used in the process layer and a contaminant gas is used in the containment vessel, the control mechanism including a piping system to divert process gas to the interior of the containment vessel in the event the pressure provided by the containment gas decreases.

6. The apparatus of claim 1 wherein an exoskeleton is mounted on the exterior of the stack to provide structural support for the stack.

7. The apparatus of claim 1 wherein end plates are attached to each side of the stack to provide structural support for the stack.

8. The apparatus of claim 1 wherein the process layer comprises at least one process microchannel for conducting a unit operation, and the heat exchange layer comprises at least one channel for a heat exchange fluid, wherein the heat exchange fluid provides heating or cooling for the process layer.

9. The apparatus of claim 1 wherein the process layer comprises a plurality of process microchannels formed in a plate, the apparatus including internal welding to prevent the flow of fluid from one process microchannel to another process microchannel in the same plate.

10. The apparatus of claim 1 wherein the heat exchange layer comprises a plurality of heat exchange channels formed in a plate, the apparatus including internal welding to prevent the flow of fluid from one heat exchange channel to another heat exchange channel in the same plate.

11. The apparatus of claim 1 wherein the apparatus further comprises: an inlet process manifold welded to the stack to provide for the flow of fluid into the process layer; an outlet process manifold welded to the stack to provide for the flow of fluid out of the process layer; at least one inlet heat exchange manifold welded to the stack to provide for the flow of fluid into the heat exchange layer; and a heat exchange outlet to provide for the flow of fluid out of the heat exchange layer.

12. The apparatus of claim 11 wherein the heat exchange outlet comprises an exhaust outlet welded to an end of the stack and adapted to provide for the flow of exhaust gas from the heat exchange layer.

13. The apparatus of claim 1 wherein a welding material is used to weld the peripheral edge of each plate, the plates being made of a metal or metal alloy, and the welding material being made of a metal or metal alloy.

14. The apparatus of claim 13 wherein the plates and the welding material are made of the same metal or metal alloy.

15. The apparatus of claim 14 wherein the metal alloy comprises nickel, chromium, cobalt, molybdenum and aluminum.

16. The apparatus of claim 1 wherein the peripheral edge of each plate is welded to the peripheral edge of the next adjacent plate using a laser.

17. The apparatus of claim 1 wherein the apparatus is suitable for conducing at least one unit operation in the process layer.

18. The apparatus of claim 17 wherein the unit operation comprises a chemical reaction, vaporization, compression, chemical separation, distillation, condensation, mixing, heating, cooling, or a combination of two or more thereof.

19. The apparatus of claim 18 wherein the chemical reaction comprises a methanol synthesis reaction, dimethyl ether synthesis reaction, ammonia synthesis reaction, water gas shift reaction, acetylation addition reaction, alkylation, dealkylation, hydrodealkylation, reductive alkylation, amination, aromatization, arylation, autothermal reforming, carbonylation, decarbonylation, reductive carbonylation, carboxylation, reductive carboxylation, reductive coupling, condensation, cracking, hydrocracking, cyclization, cyclooligomerization, dehalogenation, dimerization, epoxidation, esterification, Fischer-Tropsch reaction, halogenation, hydrohalogenation, homologation, hydration, dehydration, hydrogenation, dehydrogenation, hydrocarboxylation, hydroformylation, hydrogenolysis, hydrometallation, hydrosilation, hydrolysis, hydrotreating, isomerization, methylation, demethylation, metathesis, nitration, oxidation, partial oxidation, polymerization, reduction, reformation, reverse water gas shift, sulfonation, telomerization, transesterification, trimerization, Sabatier reaction, carbon dioxide reforming, preferential oxidation, partial oxidation, or preferential methanation reaction.

20. The apparatus of claim 18 wherein the chemical reaction comprises a steam methane reforming reaction.

21. The apparatus of claim 18 wherein the chemical reaction comprises a process for making ethylene, styrene, formaldehyde and/or butadiene.

22. The apparatus of claim 1 wherein the process layer comprises a plurality of process microchannels aligned in parallel.

23. The apparatus of claim 22 wherein each process microchannel comprises a reaction zone containing a catalyst.

24. The apparatus of claim 22 wherein the process layer comprises a plurality of internal manifolds adapted to provide for a substantially uniform distribution of reactants flowing into the process microchannels and/or a plurality of internal manifolds adapted to provide for a substantially uniform distribution of product flowing out of the process microchannels.

25. The apparatus of claim 22 wherein the process microchannels contain surface features and/or capillary features.

26. The apparatus of claim 1 wherein the process layer comprises a reactant layer and a product layer, the product layer being positioned adjacent to the reactant layer, and a process u-turn positioned at an end of the reactant layer and product layer to allow for the flow of fluid from the reactant layer to the product layer; the process layer being adapted for use in a reaction wherein one or more reactants react to form a product, the one or more reactants flowing into the reactant layer, contacting a catalyst and reacting to form a product, the product flowing out of the product layer.

27. The apparatus of claim 1 wherein the heat exchange layer is adapted to provide for the flow of a heat exchange fluid in the heat exchange layer.

28. The apparatus of claim 27 wherein the heat exchange fluid comprises a liquid, a gas, or a mixture thereof.

29. The apparatus of claim 27 wherein the heat exchange fluid comprises air, steam, liquid water, carbon monoxide, carbon dioxide, gaseous nitrogen, liquid nitrogen, inert gas, gaseous hydrocarbon, liquid hydrocarbon, or a mixture of two or more thereof.

30. The apparatus of claim 1 wherein the heat exchange layer is adapted to provide for the conducting of a combustion reaction in the heat exchange layer.

31. The apparatus of claim 1 wherein the heat exchange layer comprises a plurality of heat exchange channels aligned in parallel.

32. The apparatus of claim 1 wherein the heat exchange layer comprises a fuel layer, an air layer positioned adjacent to the fuel layer, a heat exchange wall positioned between the fuel layer and the air layer, a plurality of openings in the heat exchange wall to allow for the flow of air from the air layer through the openings into the fuel layer, a combustion catalyst positioned in the fuel layer, an exhaust layer, and a heat exchange u-turn positioned at an end of the fuel layer and an end of the exhaust layer to allow for the flow of fluid from the fuel layer to the exhaust layer; the heat exchange layer being adapted to allow for a fuel to flow in the fuel layer, air to flow from the air layer through the openings in the heat exchange wall into the fuel layer to combine with the fuel to form a fuel-air mixture, flowing the fuel-air mixture in contact with the combustion catalyst to provide for a combustion reaction to yield heat and an exhaust gas, the heat providing heat for the process layer, the exhaust gas flowing through the exhaust layer out of the heat exchange layer.

33. The apparatus of claim 1 wherein the heat exchange layer comprises a fuel layer, the fuel layer comprising a plurality of fuel microchannels and a plurality of internal manifolds adapted to provide for a substantially uniform distribution of fuel flowing into the fuel microchannels.

34. The apparatus of claim 1 wherein the heat exchange layer comprises an air layer, the air layer comprising a plurality of air microchannels and a plurality of internal manifolds adapted to provide for a substantially uniform distribution of air flowing into the air microchannels.

35. The apparatus of claim 33 wherein the fuel layer contains surface features and/or capillary features.

36. The apparatus of claim 34 wherein the air layer contains surface features and/or capillary features.

37. The apparatus of claim 1 wherein the process layer and/or the heat exchange layer are adapted for operation at an internal gauge pressure up to about 15 MPa.

38. The apparatus of claim 1 wherein the process layer and the heat exchange layer are adapted to be operated at different internal pressures, the difference in pressure between the internal pressure in the process layer and the internal pressure in the heat exchange layer being up to about 10 MPa.

39. A process for forming the apparatus of claim 1 comprising:
forming the stack of plates; and
welding the peripheral edge of each plate to the peripheral edge of the next adjacent plate to provide the perimeter seal.

40. A process for refurbishing the apparatus of claim 1, comprising:
removing the welding from the peripheral edges of the plates;
separating the plates;
correcting defects in the plates;
reforming the stack of plates; and
welding the peripheral edge of each plate to the peripheral edge of the next adjacent plate to provide a new perimeter seal for the stack.

41. The process of claim 40 wherein the apparatus contains one or more catalysts, the catalysts being replaced and/or regenerated prior to reforming the stack of plates.

42. A process for conducting a unit operation using the apparatus of claim 1, comprising:
conducting the unit operation in the process layer; and
exchanging heat between the process layer and the heat exchange layer.

43. The process of claim 42 wherein the unit operation comprises a chemical reaction, vaporization, compression, chemical separation, distillation, condensation, mixing, heating, cooling, or a combination of two or more thereof.

44. A process for conducting a chemical reaction using the apparatus of claim 1, comprising:
conducting the chemical reaction in the process layer; and
exchanging heat between the process layer and the heat exchange layer.

45. The process of claim 44 wherein the chemical reaction comprises a methanol synthesis reaction, dimethyl ether synthesis reaction, ammonia synthesis reaction, water gas shift reaction, acetylation addition reaction, alkylation, dealkylation, hydrodealkylation, reductive alkylation, amination, aromatization, arylation, autothermal reforming, carbonylation, decarbonylation, reductive carbonylation, carboxylation, reductive carboxylation, reductive coupling, condensation, cracking, hydrocracking, cyclization, cyclooligomerization, dehalogenation, dimerization, epoxidation, esterification, Fischer-Tropsch reaction, halogenation, hydrohalogenation, homologation, hydration, dehydration, hydrogenation, dehydrogenation, hydrocarboxylation, hydroformylation, hydrogenolysis, hydrometallation, hydrosilation, hydrolysis, hydrotreating, isomerization, methylation, demethylation, metathesis, nitration, oxidation, partial oxidation, polymerization, reduction, reformation, reverse water gas shift, sulfonation, telomerization, transesterification, trimerization, Sabatier reaction, carbon dioxide reforming, preferential oxidation, partial oxidation, or preferential methanation reaction.

46. The process of claim 44 wherein the chemical reaction comprises a process for making ethylene, styrene, formaldehyde and/or butadiene.

47. A process for conducting a steam methane reforming reaction using the apparatus of claim 1, comprising:
reacting steam with methane or natural gas in the presence of a catalyst in the process layer to form synthesis gas; and
conducting a combustion reaction in the heat exchange layer to provide heat for the process layer.

48. The apparatus of claim 1 wherein the apparatus comprises a steam methane reforming reactor, the process layer containing a steam methane reforming catalyst, the heat exchange layer containing a combustion catalyst.

49. The apparatus of claim 48 wherein the steam methane reforming catalyst comprises rhodium and an alumina support.

50. The apparatus of claim 48 wherein the combustion catalyst comprises platinum, palladium, and an alumina support, the alumina support being impregnated with lanthanum.

51. The apparatus of claim 1 wherein a catalyst is present in the process layer and/or the heat exchange layer, the catalyst being applied to one or more plates ex-situ prior to welding the plates to form the stack.

52. The apparatus of claim 1 wherein one or more of the plates have an anti-corrosion and/or anti-sticking layer on one or more surfaces of such plates.

53. The apparatus of claim 1 wherein one or more of the plates have a metal dust resistant layer on one or more surfaces of such plates.

54. The apparatus of claim 1 wherein a plate in the process layer and/or heat exchange layer comprises a surface wherein part but not all of the surface has a catalyst, anti-corrosion and/or anti-sticking layer, and/or metal dust resistant layer on it.

55. The apparatus of claim 1 wherein one or more of the plates has one or more surface protection layers on it.

56. The apparatus of claim 55 wherein the surface protection layer comprises two or three layers, each layer comprising a different composition of materials.

57. The apparatus of claim 55 wherein the surface protection layer comprises three layers, the first layer comprising copper, the second layer comprising an aluminum-containing metal alloy, and the third layer comprising a metal alloy.

58. The apparatus of claim 55 wherein a catalyst is adhered to the surface protection layer.

59. The process of claim 40 wherein the apparatus contains one or more catalysts, the one or more catalysts being adhered to one or more surfaces of one or more of the plates, the catalyst being removed by grit blasting.

60. The process of claim 40 wherein one or more of the plates has an alumina scale on one or more of its surfaces that is damaged, the alumina scale being replenished by heat treating.

61. The process of claim 47 wherein the flow of methane or natural gas in the process layer is at a superficial velocity in the range of about 10 to about 200 meters per second, the approach to equilibrium for the steam methane reforming reaction being at least about 80%, and the reaction heat per pressure drop in the apparatus being in the range from about 2 to about 20 W/Pa.

62. The process of claim 47 wherein the contact time for the steam methane reforming reaction is up to about 25 ms, the approach to equilibrium for the steam methane reforming reaction being at least about 80%, and the reaction heat per pressure drop in the apparatus being in the range from about 2 to about 20 W/Pa.

63. The process of claim 61 wherein the reaction heat per unit contact time is at least about 20 W/ms.

64. The process of claim 47 wherein the steam methane reforming reaction is conducted for at least about 2000 hours without metal dusting pits forming on surfaces of the plates.

65. The process of claim 47 wherein the steam methane reforming reaction is conducted for at least about 2000 hours and the pressure drop for the process layer after conducting the reaction for at least about 2000 hours increases by less than about 20% of the pressure drop at the start of the process.

66. A refurbished apparatus made by the process of claim 40.
